SEQUENCE LISTING (199 sequences)

SEQ ID NO: 1 - DNA, 32 bp, Artificial Sequence
  Synthetic Polynucleotide
taaatgaattttctgtatgggattaatttctt

SEQ ID NO: 2 - DNA, 31 bp, Artificial Sequence
  Synthetic Polynucleotide
tctaaagttttgtcgtctttccagccgacaa

SEQ ID NO: 3 - DNA, 32 bp, Artificial Sequence
  Synthetic Polynucleotide
tccacagacagccctcatagttagcgtaacga

SEQ ID NO: 4 - DNA, 32 bp, Artificial Sequence
  Synthetic Polynucleotide
tcaccagtacaaactacaacgcctagtaccag

SEQ ID NO: 5 - DNA, 31 bp, Artificial Sequence
  Synthetic Polynucleotide
aggaacccatgtaccgtaacacttgatataa

SEQ ID NO: 6 - DNA, 32 bp, Artificial Sequence
  Synthetic Polynucleotide
ccaccctcattttcagggatagcaaccgtact

SEQ ID NO: 7 - DNA, 31 bp, Artificial Sequence
  Synthetic Polynucleotide
agaaaggaacaactaaaggaattcaaaaaaa

SEQ ID NO: 8 - DNA, 32 bp, Artificial Sequence
  Synthetic Polynucleotide
acaactttcaacagtttcagcggatgtatcgg

SEQ ID NO: 9 - DNA, 44 bp, Artificial Sequence
  Synthetic Polynucleotide
tgacaactcgctgaggcttgcattataccaagcgcgatgataaa

SEQ ID NO: 10 - DNA, 32 bp, Artificial Sequence
  Synthetic Polynucleotide
ttaggattggctgagactcctcaataaccgat

SEQ ID NO: 11 - DNA, 48 bp, Artificial Sequence
  Synthetic Polynucleotide
gcggataacctattattctgaaacagacgattggccttgaagagccac

SEQ ID NO: 12 - DNA, 30 bp, Artificial Sequence
  Synthetic Polynucleotide
gtatagcaaacagttaatgcccaatcctca

SEQ ID NO: 13 - DNA, 48 bp, Artificial Sequence
  Synthetic Polynucleotide
caggaggtggggtcagtgccttgagtctctgaatttaccgggaaccag

SEQ ID NO: 14 - DNA, 30 bp, Artificial Sequence
  Synthetic Polynucleotide
aggctccagaggctttgaggacacgggtaa

SEQ ID NO: 15 - DNA, 48 bp, Artificial Sequence
  Synthetic Polynucleotide
tttatcaggacagcatcggaacgacaccaacctaaaacgaggtcaatc

SEQ ID NO: 16 - DNA, 32 bp, Artificial Sequence
  Synthetic Polynucleotide
aaacagctttttgcgggatcgtcaacactaaa

SEQ ID NO: 17 - DNA, 32 bp, Artificial Sequence
  Synthetic Polynucleotide
ttgctcctttcaaatatcgcgtttgagggggt

SEQ ID NO: 18 - DNA, 32 bp, Artificial Sequence
  Synthetic Polynucleotide
ccaacaggagcgaaccagaccggagcctttac

SEQ ID NO: 19 - DNA, 30 bp, Artificial Sequence
  Synthetic Polynucleotide
ttaacgtctaacataaaaacaggtaacgga

SEQ ID NO: 20 - DNA, 32 bp, Artificial Sequence
  Synthetic Polynucleotide
atcccaatgagaattaactgaacagttaccag

SEQ ID NO: 21 - DNA, 32 bp, Artificial Sequence
  Synthetic Polynucleotide
gccagttagagggtaattgagcgctttaagaa

SEQ ID NO: 22 - DNA, 30 bp, Artificial Sequence
  Synthetic Polynucleotide
acgctaacacccacaagaattgaaaatagc

SEQ ID NO: 23 - DNA, 32 bp, Artificial Sequence
  Synthetic Polynucleotide
ctgtagcttgactattatagtcagttcattga

SEQ ID NO: 24 - DNA, 30 bp, Artificial Sequence
  Synthetic Polynucleotide
gatggcttatcaaaaagattaagagcgtcc

SEQ ID NO: 25 - DNA, 30 bp, Artificial Sequence
  Synthetic Polynucleotide
tttggggatagtagtagcattaaaaggccg

SEQ ID NO: 26 - DNA, 32 bp, Artificial Sequence
  Synthetic Polynucleotide
ccaatagctcatcgtaggaatcatggcatcaa

SEQ ID NO: 27 - DNA, 32 bp, Artificial Sequence
  Synthetic Polynucleotide
tatccggtctcatcgagaacaagcgacaaaag

SEQ ID NO: 28 - DNA, 30 bp, Artificial Sequence
  Synthetic Polynucleotide
gcgaacctccaagaacgggtatgacaataa

SEQ ID NO: 29 - DNA, 32 bp, Artificial Sequence
  Synthetic Polynucleotide
gccttaaaccaatcaataatcggcacgcgcct

SEQ ID NO: 30 - DNA, 30 bp, Artificial Sequence
  Synthetic Polynucleotide
aacagttttgtaccaaaaacattttatttc

SEQ ID NO: 31 - DNA, 32 bp, Artificial Sequence
  Synthetic Polynucleotide
gatttagtcaataaagcctcagagaaccctca

SEQ ID NO: 32 - DNA, 32 bp, Artificial Sequence
  Synthetic Polynucleotide
aatggtcaacaggcaaggcaaagagtaatgtg

SEQ ID NO: 33 - DNA, 32 bp, Artificial Sequence
  Synthetic Polynucleotide
taaatcatataacctgtttagctaacctttaa

SEQ ID NO: 34 - DNA, 32 bp, Artificial Sequence
  Synthetic Polynucleotide
ttctactacgcgagctgaaaaggttaccgcgc

SEQ ID NO: 35 - DNA, 30 bp, Artificial Sequence
  Synthetic Polynucleotide
ttttatttaagcaaatcagatattttttgt

SEQ ID NO: 36 - DNA, 32 bp, Artificial Sequence
  Synthetic Polynucleotide
gtaccgcaattctaagaacgcgagtattattt

SEQ ID NO: 37 - DNA, 32 bp, Artificial Sequence
  Synthetic Polynucleotide
cttatcattcccgacttgcgggagcctaattt

SEQ ID NO: 38 - DNA, 30 bp, Artificial Sequence
  Synthetic Polynucleotide
tgtagaaatcaagattagttgctcttacca

SEQ ID NO: 39 - DNA, 32 bp, Artificial Sequence
  Synthetic Polynucleotide
taaatcgggattcccaattctgcgatataatg

SEQ ID NO: 40 - DNA, 30 bp, Artificial Sequence
  Synthetic Polynucleotide
aaattaagttgaccattagatacttttgcg

SEQ ID NO: 41 - DNA, 30 bp, Artificial Sequence
  Synthetic Polynucleotide
gagacagctagctgataaattaatttttgt

SEQ ID NO: 42 - DNA, 32 bp, Artificial Sequence
  Synthetic Polynucleotide
gtaataagttaggcagaggcatttatgatatt

SEQ ID NO: 43 - DNA, 32 bp, Artificial Sequence
  Synthetic Polynucleotide
gtaaagtaatcgccatatttaacaaaactttt

SEQ ID NO: 44 - DNA, 30 bp, Artificial Sequence
  Synthetic Polynucleotide
acaacatgccaacgctcaacagtcttctga

SEQ ID NO: 45 - DNA, 32 bp, Artificial Sequence
  Synthetic Polynucleotide
gtttatcaatatgcgttatacaaaccgaccgt

SEQ ID NO: 46 - DNA, 30 bp, Artificial Sequence
  Synthetic Polynucleotide
aacgcaaaatcgatgaacggtaccggttga

SEQ ID NO: 47 - DNA, 32 bp, Artificial Sequence
  Synthetic Polynucleotide
tatattttgtcattgcctgagagtggaagatt

SEQ ID NO: 48 - DNA, 32 bp, Artificial Sequence
  Synthetic Polynucleotide
taggtaaactatttttgagagatcaaacgtta

SEQ ID NO: 49 - DNA, 32 bp, Artificial Sequence
  Synthetic Polynucleotide
gagggtaggattcaaaagggtgagacatccaa

SEQ ID NO: 50 - DNA, 32 bp, Artificial Sequence
  Synthetic Polynucleotide
caaccgtttcaaatcaccatcaattcgagcca

SEQ ID NO: 51 - DNA, 30 bp, Artificial Sequence
  Synthetic Polynucleotide
catgtaatagaatataaagtaccaagccgt

SEQ ID NO: 52 - DNA, 32 bp, Artificial Sequence
  Synthetic Polynucleotide
aattgagaattctgtccagacgactaaaccaa

SEQ ID NO: 53 - DNA, 32 bp, Artificial Sequence
  Synthetic Polynucleotide
agtataaagttcagctaatgcagatgtctttc

SEQ ID NO: 54 - DNA, 30 bp, Artificial Sequence
  Synthetic Polynucleotide
ttagtatcacaatagataagtccacgagca

SEQ ID NO: 55 - DNA, 32 bp, Artificial Sequence
  Synthetic Polynucleotide
aacaagagggataaaaatttttagcataaagc

SEQ ID NO: 56 - DNA, 30 bp, Artificial Sequence
  Synthetic Polynucleotide
gctatcagaaatgcaatgcctgaattagca

SEQ ID NO: 57 - DNA, 44 bp, Artificial Sequence
  Synthetic Polynucleotide
taaatcaaaataattcgcgtctcggaaaccaggcaaagggaagg

SEQ ID NO: 58 - DNA, 32 bp, Artificial Sequence
  Synthetic Polynucleotide
atcgcaagtatgtaaatgctgatgataggaac

SEQ ID NO: 59 - DNA, 48 bp, Artificial Sequence
  Synthetic Polynucleotide
tcaaatataacctccggcttaggtaacaatttcatttgaaggcgaatt

SEQ ID NO: 60 - DNA, 30 bp, Artificial Sequence
  Synthetic Polynucleotide
cctaaatcaaaatcataggtctaaacagta

SEQ ID NO: 61 - DNA, 48 bp, Artificial Sequence
  Synthetic Polynucleotide
gtgataaaaagacgctgagaagagataaccttgcttctgttcgggaga

SEQ ID NO: 62 - DNA, 30 bp, Artificial Sequence
  Synthetic Polynucleotide
taatcagcggattgaccgtaatcgtaaccg

SEQ ID NO: 63 - DNA, 48 bp, Artificial Sequence
  Synthetic Polynucleotide
gtataagccaacccgtcggattctgacgacagtatcggccgcaaggcg

SEQ ID NO: 64 - DNA, 32 bp, Artificial Sequence
  Synthetic Polynucleotide
atattttggctttcatcaacattatccagcca

SEQ ID NO: 65 - DNA, 32 bp, Artificial Sequence
  Synthetic Polynucleotide
tgtagccattaaaattcgcattaaatgccgga

SEQ ID NO: 66 - DNA, 32 bp, Artificial Sequence
  Synthetic Polynucleotide
gccatcaagctcattttttaaccacaaatcca

SEQ ID NO: 67 - DNA, 30 bp, Artificial Sequence
  Synthetic Polynucleotide
tataactaacaaagaacgcgagaacgccaa

SEQ ID NO: 68 - DNA, 32 bp, Artificial Sequence
  Synthetic Polynucleotide
acctttttattttagttaatttcatagggctt

SEQ ID NO: 69 - DNA, 32 bp, Artificial Sequence
  Synthetic Polynucleotide
gaatttatttaatggtttgaaatattcttacc

SEQ ID NO: 70 - DNA, 30 bp, Artificial Sequence
  Synthetic Polynucleotide
cttagatttaaggcgttaaataaagcctgt

SEQ ID NO: 71 - DNA, 32 bp, Artificial Sequence
  Synthetic Polynucleotide
acaaacggaaaagccccaaaaacactggagca

SEQ ID NO: 72 - DNA, 30 bp, Artificial Sequence
  Synthetic Polynucleotide
gcgagtaaaaatatttaaattgttacaaag

SEQ ID NO: 73 - DNA, 32 bp, Artificial Sequence
  Synthetic Polynucleotide
agaaaacaaagaagatgatgaaacaggctgcg

SEQ ID NO: 74 - DNA, 30 bp, Artificial Sequence
  Synthetic Polynucleotide
cataaatctttgaataccaagtgttagaac

SEQ ID NO: 75 - DNA, 30 bp, Artificial Sequence
  Synthetic Polynucleotide
tgcatctttcccagtcacgacggcctgcag

SEQ ID NO: 76 - DNA, 32 bp, Artificial Sequence
  Synthetic Polynucleotide
gctttccgattacgccagctggcggctgtttc

SEQ ID NO: 77 - DNA, 32 bp, Artificial Sequence
  Synthetic Polynucleotide
tcttcgctgcaccgcttctggtgcggccttcc

SEQ ID NO: 78 - DNA, 32 bp, Artificial Sequence
  Synthetic Polynucleotide
caactgttgcgccattcgccattcaaacatca

SEQ ID NO: 79 - DNA, 30 bp, Artificial Sequence
  Synthetic Polynucleotide
ctgagcaaaaattaattacattttgggtta

SEQ ID NO: 80 - DNA, 32 bp, Artificial Sequence
  Synthetic Polynucleotide
cgcgcagattaccttttttaatgggagagact

SEQ ID NO: 81 - DNA, 32 bp, Artificial Sequence
  Synthetic Polynucleotide
cctgattgcaatatatgtgagtgatcaatagt

SEQ ID NO: 82 - DNA, 30 bp, Artificial Sequence
  Synthetic Polynucleotide
cttttacaaaatcgtcgctattagcgatag

SEQ ID NO: 83 - DNA, 32 bp, Artificial Sequence
  Synthetic Polynucleotide
ccagggttgccagtttgaggggacccgtggga

SEQ ID NO: 84 - DNA, 30 bp, Artificial Sequence
  Synthetic Polynucleotide
gatgtgcttcaggaagatcgcacaatgtga

SEQ ID NO: 85 - DNA, 32 bp, Artificial Sequence
  Synthetic Polynucleotide
gcaattcacatattcctgattatcaaagtgta

SEQ ID NO: 86 - DNA, 30 bp, Artificial Sequence
  Synthetic Polynucleotide
ctaccatagtttgagtaacatttaaaatat

SEQ ID NO: 87 - DNA, 30 bp, Artificial Sequence
  Synthetic Polynucleotide
gtcgacttcggccaacgcgcggggtttttc

SEQ ID NO: 88 - DNA, 32 bp, Artificial Sequence
  Synthetic Polynucleotide
ctgtgtgattgcgttgcgctcactagagttgc

SEQ ID NO: 89 - DNA, 32 bp, Artificial Sequence
  Synthetic Polynucleotide
aaggccgctgataccgatagttgcgacgttag

SEQ ID NO: 90 - DNA, 32 bp, Artificial Sequence
  Synthetic Polynucleotide
atattcggaaccatcgcccacgcagagaagga

SEQ ID NO: 91 - DNA, 30 bp, Artificial Sequence
  Synthetic Polynucleotide
tattaagaagcggggttttgctcgtagcat

SEQ ID NO: 92 - DNA, 32 bp, Artificial Sequence
  Synthetic Polynucleotide
tttcggaagtgccgtcgagagggtgagtttcg

SEQ ID NO: 93 - DNA, 32 bp, Artificial Sequence
  Synthetic Polynucleotide
gcccgtatccggaataggtgtatcagcccaat

SEQ ID NO: 94 - DNA, 30 bp, Artificial Sequence
  Synthetic Polynucleotide
gttttaacttagtaccgccacccagagcca

SEQ ID NO: 95 - DNA, 32 bp, Artificial Sequence
  Synthetic Polynucleotide
acggctacaaaaggagcctttaatgtgagaat

SEQ ID NO: 96 - DNA, 30 bp, Artificial Sequence
  Synthetic Polynucleotide
cagcgaaacttgctttcgaggtgttgctaa

SEQ ID NO: 97 - DNA, 32 bp, Artificial Sequence
  Synthetic Polynucleotide
cacattaaaattgttatccgctcatgcgggcc

SEQ ID NO: 98 - DNA, 32 bp, Artificial Sequence
  Synthetic Polynucleotide
aagcctggtacgagccggaagcatagatgatg

SEQ ID NO: 99 - DNA, 30 bp, Artificial Sequence
  Synthetic Polynucleotide
attatcattcaatataatcctgacaattac

SEQ ID NO: 100 - DNA, 32 bp, Artificial Sequence
  Synthetic Polynucleotide
gcggaacatctgaataatggaaggtacaaaat

SEQ ID NO: 101 - DNA, 32 bp, Artificial Sequence
  Synthetic Polynucleotide
attttaaaatcaaaattatttgcacggattcg

SEQ ID NO: 102 - DNA, 30 bp, Artificial Sequence
  Synthetic Polynucleotide
ctcgtattagaaattgcgtagatacagtac

SEQ ID NO: 103 - DNA, 32 bp, Artificial Sequence
  Synthetic Polynucleotide
ttaatgaactagaggatccccggggggtaacg

SEQ ID NO: 104 - DNA, 30 bp, Artificial Sequence
  Synthetic Polynucleotide
ttccagtcgtaatcatggtcataaaagggg

SEQ ID NO: 105 - DNA, 38 bp, Artificial Sequence
  Synthetic Polynucleotide
cccagcaggcgaaaaatcccttataaatcaagccggcg

SEQ ID NO: 106 - DNA, 32 bp, Artificial Sequence
  Synthetic Polynucleotide
tcaatatcgaacctcaaatatcaattccgaaa

SEQ ID NO: 107 - DNA, 40 bp, Artificial Sequence
  Synthetic Polynucleotide
tcaacagttgaaaggagcaaatgaaaaatctagagataga

SEQ ID NO: 108 - DNA, 30 bp, Artificial Sequence
  Synthetic Polynucleotide
ctttagggcctgcaacagtgccaatacgtg

SEQ ID NO: 109 - DNA, 40 bp, Artificial Sequence
  Synthetic Polynucleotide
agattagagccgtcaaaaaacagaggtgaggcctattagt

SEQ ID NO: 110 - DNA, 30 bp, Artificial Sequence
  Synthetic Polynucleotide
ttttcactcaaagggcgaaaaaccatcacc

SEQ ID NO: 111 - DNA, 40 bp, Artificial Sequence
  Synthetic Polynucleotide
agctgattgcccttcagagtccactattaaagggtgccgt

SEQ ID NO: 112 - DNA, 32 bp, Artificial Sequence
  Synthetic Polynucleotide
agcaagcgtagggttgagtgttgtagggagcc

SEQ ID NO: 113 - DNA, 32 bp, Artificial Sequence
  Synthetic Polynucleotide
gcccgagagtccacgctggtttgcagctaact

SEQ ID NO: 114 - DNA, 32 bp, Artificial Sequence
  Synthetic Polynucleotide
tcggcaaatcctgtttgatggtggaccctcaa

SEQ ID NO: 115 - DNA, 30 bp, Artificial Sequence
  Synthetic Polynucleotide
accttgcttggtcagttggcaaagagcgga

SEQ ID NO: 116 - DNA, 32 bp, Artificial Sequence
  Synthetic Polynucleotide
agccagcaattgaggaaggttatcatcatttt

SEQ ID NO: 117 - DNA, 32 bp, Artificial Sequence
  Synthetic Polynucleotide
ttaacaccagcactaacaactaatcgttatta

SEQ ID NO: 118 - DNA, 30 bp, Artificial Sequence
  Synthetic Polynucleotide
cagaagattagataatacatttgtcgacaa

SEQ ID NO: 119 - DNA, 32 bp, Artificial Sequence
  Synthetic Polynucleotide
ctccaacgcagtgagacgggcaaccagctgca

SEQ ID NO: 120 - DNA, 30 bp, Artificial Sequence
  Synthetic Polynucleotide
tggaacaaccgcctggccctgaggcccgct

SEQ ID NO: 121 - DNA, 31 bp, Artificial Sequence
  Synthetic Polynucleotide
aacgtggcgagaaaggaagggaaaccagtaa

SEQ ID NO: 122 - DNA, 30 bp, Artificial Sequence
  Synthetic Polynucleotide
taaaagggacattctggccaacaaagcatc

SEQ ID NO: 123 - DNA, 32 bp, Artificial Sequence
  Synthetic Polynucleotide
acccttctgacctgaaagcgtaagacgctgag

SEQ ID NO: 124 - DNA, 31 bp, Artificial Sequence
  Synthetic Polynucleotide
gcacagacaatatttttgaatggggtcagta

SEQ ID NO: 125 - DNA, 30 bp, Artificial Sequence
  Synthetic Polynucleotide
ctttaatgcgcgaactgatagccccaccag

SEQ ID NO: 126 - DNA, 31 bp, Artificial Sequence
  Synthetic Polynucleotide
caaatcaagttttttggggtcgaaacgtgga

SEQ ID NO: 127 - DNA, 30 bp, Artificial Sequence
  Synthetic Polynucleotide
aaagcactaaatcggaaccctaatccagtt

SEQ ID NO: 128 - DNA, 32 bp, Artificial Sequence
  Synthetic Polynucleotide
cccgatttagagcttgacggggaaaaagaata

SEQ ID NO: 129 - DNA, 32 bp, Artificial Sequence
  Synthetic Polynucleotide
ttgacaggccaccaccagagccgcgatttgta

SEQ ID NO: 130 - DNA, 30 bp, Artificial Sequence
  Synthetic Polynucleotide
ttaaagccagagccgccaccctcgacagaa

SEQ ID NO: 131 - DNA, 30 bp, Artificial Sequence
  Synthetic Polynucleotide
aatacgtttgaaagaggacagactgacctt

SEQ ID NO: 132 - DNA, 32 bp, Artificial Sequence
  Synthetic Polynucleotide
acactcatccatgttacttagccgaaagctgc

SEQ ID NO: 133 - DNA, 32 bp, Artificial Sequence
  Synthetic Polynucleotide
gacctgctctttgacccccagcgagggagtta

SEQ ID NO: 134 - DNA, 32 bp, Artificial Sequence
  Synthetic Polynucleotide
tcatcgccaacaaagtacaacggacgccagca

SEQ ID NO: 135 - DNA, 30 bp, Artificial Sequence
  Synthetic Polynucleotide
caccagaaaggttgaggcaggtcatgaaag

SEQ ID NO: 136 - DNA, 32 bp, Artificial Sequence
  Synthetic Polynucleotide
ccaccctctattcacaaacaaatacctgccta

SEQ ID NO: 137 - DNA, 32 bp, Artificial Sequence
  Synthetic Polynucleotide
gcctccctcagaatggaaagcgcagtaacagt

SEQ ID NO: 138 - DNA, 30 bp, Artificial Sequence
  Synthetic Polynucleotide
aaatcaccttccagtaagcgtcagtaataa

SEQ ID NO: 139 - DNA, 32 bp, Artificial Sequence
  Synthetic Polynucleotide
gaccaactaatgccactacgaagggggtagca

SEQ ID NO: 140 - DNA, 30 bp, Artificial Sequence
  Synthetic Polynucleotide
gcgcagacaagaggcaaaagaatccctcag

SEQ ID NO: 141 - DNA, 32 bp, Artificial Sequence
  Synthetic Polynucleotide
gcaaggcctcaccagtagcaccatgggcttga

SEQ ID NO: 142 - DNA, 30 bp, Artificial Sequence
  Synthetic Polynucleotide
tcaagtttcattaaaggtgaatataaaaga

SEQ ID NO: 143 - DNA, 30 bp, Artificial Sequence
  Synthetic Polynucleotide
catcaagtaaaacgaactaacgagttgaga

SEQ ID NO: 144 - DNA, 32 bp, Artificial Sequence
  Synthetic Polynucleotide
tcattcagatgcgattttaagaacaggcatag

SEQ ID NO: 145 - DNA, 32 bp, Artificial Sequence
  Synthetic Polynucleotide
attacctttgaataaggcttgcccaaatccgc

SEQ ID NO: 146 - DNA, 32 bp, Artificial Sequence
  Synthetic Polynucleotide
gatggtttgaacgagtagtaaatttaccatta

SEQ ID NO: 147 - DNA, 30 bp, Artificial Sequence
  Synthetic Polynucleotide
cagcaaaaggaaacgtcaccaatgagccgc

SEQ ID NO: 148 - DNA, 32 bp, Artificial Sequence
  Synthetic Polynucleotide
tcaccgacgcaccgtaatcagtagcagaaccg

SEQ ID NO: 149 - DNA, 32 bp, Artificial Sequence
  Synthetic Polynucleotide
gaaattattgcctttagcgtcagaccggaacc

SEQ ID NO: 150 - DNA, 30 bp, Artificial Sequence
  Synthetic Polynucleotide
accgattgtcggcattttcggtcataatca

SEQ ID NO: 151 - DNA, 32 bp, Artificial Sequence
  Synthetic Polynucleotide
tacgttaaagtaatcttgacaagaaccgaact

SEQ ID NO: 152 - DNA, 30 bp, Artificial Sequence
  Synthetic Polynucleotide
ttataccaccaaatcaacgtaacgaacgag

SEQ ID NO: 153 - DNA, 38 bp, Artificial Sequence
  Synthetic Polynucleotide
cgtttaccagacgacaaagaagttttgccataattcga

SEQ ID NO: 154 - DNA, 32 bp, Artificial Sequence
  Synthetic Polynucleotide
ttattacgaagaactggcatgattgcgagagg

SEQ ID NO: 155 - DNA, 40 bp, Artificial Sequence
  Synthetic Polynucleotide
cgtagaaaatacataccgaggaaacgcaataagaagcgca

SEQ ID NO: 156 - DNA, 30 bp, Artificial Sequence
  Synthetic Polynucleotide
aacgcaaagatagccgaacaaaccctgaac

SEQ ID NO: 157 - DNA, 40 bp, Artificial Sequence
  Synthetic Polynucleotide
gtttattttgtcacaatcttaccgaagccctttaatatca

SEQ ID NO: 158 - DNA, 30 bp, Artificial Sequence
  Synthetic Polynucleotide
tttaggacaaatgctttaaacaatcaggtc

SEQ ID NO: 159 - DNA, 40 bp, Artificial Sequence
  Synthetic Polynucleotide
atgcagatacataacgggaatcgtcataaataaagcaaag

SEQ ID NO: 160 - DNA, 32 bp, Artificial Sequence
  Synthetic Polynucleotide
taagagcaaatgtttagactggataggaagcc

SEQ ID NO: 161 - DNA, 32 bp, Artificial Sequence
  Synthetic Polynucleotide
aatagtaaacactatcataaccctcattgtga

SEQ ID NO: 162 - DNA, 32 bp, Artificial Sequence
  Synthetic Polynucleotide
cttttgcagataaaaaccaaaataaagactcc

SEQ ID NO: 163 - DNA, 30 bp, Artificial Sequence
  Synthetic Polynucleotide
atacccaacagtatgttagcaaattagagc

SEQ ID NO: 164 - DNA, 32 bp, Artificial Sequence
  Synthetic Polynucleotide
aaggaaacataaaggtggcaacattatcaccg

SEQ ID NO: 165 - DNA, 32 bp, Artificial Sequence
  Synthetic Polynucleotide
aagtaagcagacaccacggaataatattgacg

SEQ ID NO: 166 - DNA, 30 bp, Artificial Sequence
  Synthetic Polynucleotide
aatagctatcaatagaaaattcaacattca

SEQ ID NO: 167 - DNA, 32 bp, Artificial Sequence
  Synthetic Polynucleotide
atccccctataccacattcaactagaaaaatc

SEQ ID NO: 168 - DNA, 30 bp, Artificial Sequence
  Synthetic Polynucleotide
aatactgcccaaaaggaattacgtggctca

SEQ ID NO: 169 - DNA, 30 bp, Artificial Sequence
  Synthetic Polynucleotide
gcttcaatcaggattagagagttattttca

SEQ ID NO: 170 - DNA, 32 bp, Artificial Sequence
  Synthetic Polynucleotide
agagagaaaaaaatgaaaatagcaagcaaact

SEQ ID NO: 171 - DNA, 32 bp, Artificial Sequence
  Synthetic Polynucleotide
ttagacggccaaataagaaacgatagaaggct

SEQ ID NO: 172 - DNA, 30 bp, Artificial Sequence
  Synthetic Polynucleotide
aaagtcacaaaataaacagccagcgtttta

SEQ ID NO: 173 - DNA, 32 bp, Artificial Sequence
  Synthetic Polynucleotide
gagagatagagcgtctttccagaggttttgaa

SEQ ID NO: 174 - DNA, 30 bp, Artificial Sequence
  Synthetic Polynucleotide
tttaccccaacatgttttaaatttccatat

SEQ ID NO: 175 - DNA, 32 bp, Artificial Sequence
  Synthetic Polynucleotide
cggattgcagagcttaattgctgaaacgagta

SEQ ID NO: 176 - DNA, 32 bp, Artificial Sequence
  Synthetic Polynucleotide
cgaaagactttgataagaggtcatatttcgca

SEQ ID NO: 177 - DNA, 40 bp, Artificial Sequence
  Synthetic Polynucleotide
  misc_feature (1)..(1): A is modified with Biotin
ataagggaaccggatattcattacgtcaggacgttgggaa

SEQ ID NO: 178 - DNA, 38 bp, Artificial Sequence
  Synthetic Polynucleotide
  misc_feature (1)..(1): T is modified with Biotin
ttgtgtcgtgacgagaaacaccaaatttcaactttaat

SEQ ID NO: 179 - DNA, 40 bp, Artificial Sequence
  Synthetic Polynucleotide
  misc_feature (1)..(1): C is modified with Biotin
caccctcagaaaccatcgatagcattgagccatttgggaa

SEQ ID NO: 180 - DNA, 40 bp, Artificial Sequence
  Synthetic Polynucleotide
  misc_feature (1)..(1): A is modified with Biotin
agccaccactgtagcgcgttttcaagggagggaaggtaaa

SEQ ID NO: 181 - DNA, 40 bp, Artificial Sequence
  Synthetic Polynucleotide
  misc_feature (1)..(1): A is modified with Biotin
attaagtttaccgagctcgaattcgggaaacctgtcgtgc

SEQ ID NO: 182 - DNA, 38 bp, Artificial Sequence
  Synthetic Polynucleotide
  misc_feature (1)..(1): G is modified with Biotin
gcgatcggcaattccacacaacaggtgcctaatgagtg

SEQ ID NO: 183 - DNA, 40 bp, Artificial Sequence
  Synthetic Polynucleotide
  misc_feature (1)..(1): A is modified with Biotin
attcatttttgtttggattatactaagaaaccaccagaag

SEQ ID NO: 184 - DNA, 40 bp, Artificial Sequence
  Synthetic Polynucleotide
  misc_feature (1)..(1): A is modified with Biotin
aacaataacgtaaaacagaaataaaaatcctttgcccgaa

SEQ ID NO: 185 - DNA, 6912 bp, Artificial Sequence
  Synthetic Polynucleotide
ttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttg

SEQ ID NO: 186 - DNA, 21 bp, Artificial Sequence
  Synthetic Polynucleotide
  misc_feature (21)..(21): A is modified with Atto 647N
gtgatgtaggtggtagaggaa

SEQ ID NO: 187 - DNA, 21 bp, Artificial Sequence
  Synthetic Polynucleotide
  misc_feature (21)..(21): A is modified with Cy3
tatgagaagttaggaatgtta

SEQ ID NO: 188 - DNA, 21 bp, Artificial Sequence
  Synthetic Polynucleotide
  misc_feature (21)..(21): A is modified with Atto 488
cgagtttaggagagatggtaa

SEQ ID NO: 189 - DNA, 21 bp, Artificial Sequence
  Synthetic Polynucleotide
ttcctctaccacctacatcac

SEQ ID NO: 190 - DNA, 21 bp, Artificial Sequence
  Synthetic Polynucleotide
taacattcctaacttctcata

SEQ ID NO: 191 - DNA, 21 bp, Artificial Sequence
  Synthetic Polynucleotide
ttaccatctctcctaaactcg

SEQ ID NO: 192 - DNA, 21 bp, Artificial Sequence
  Synthetic Polynucleotide
  misc_feature (1)..(1): C is modified with Alexa 647
  misc_feature (21)..(21): T is modified with Biotin
ctcctcgcccttgctcaccat

SEQ ID NO: 193 - DNA, 21 bp, Artificial Sequence
  Synthetic Polynucleotide
atggtgagcaagggcgaggag

SEQ ID NO: 194 - DNA, 64 bp, Artificial Sequence
  Synthetic Polynucleotide
cctcacctctactcccacccacacgcaccctccctcacctctactcccacccacacgcaccctc

SEQ ID NO: 195 - DNA, 32 bp, Artificial Sequence
  Synthetic Polynucleotide
  misc_feature (1)..(1): T is modified with Cy3
tcccacccacacgcaccctccctcacctctac

SEQ ID NO: 196 - DNA, 32 bp, Artificial Sequence
  Synthetic Polynucleotide
gagggtgcgtgtgggtgggagtagaggtgagg

SEQ ID NO: 197 - DNA, 21 bp, Artificial Sequence
  Synthetic Polynucleotide
taccctatctgagtgagtagc

SEQ ID NO: 198 - DNA, 21 bp, Artificial Sequence
  Synthetic Polynucleotide
gccaacatgacactggctaag

SEQ ID NO: 199 - DNA, 45 bp, Artificial Sequence
  Synthetic Polynucleotide
atgggatagactcactcatcgtttcggttgtactgtgaccgattc

(19) United States
(12) Patent Application Publication
(10) Pub. No.: US 2017/0327888 A1
(43) Pub. Date: Nov. 16, 2017

(54) TRIGGERED ASSEMBLY OF METAFLUOROPHORES

(71) Applicant: President and Fellows of Harvard College, Cambridge, MA (US)

(72) Inventors: Luvena L. Ong, Cambridge, MA (US); David Yu Zhang, Houston, TX (US); Diming Wei, Cambridge, MA (US); Ralf Jungmann, Munich (DE); Peng Yin, Brookline, MA (US)

(73) Assignee: President and Fellows of Harvard College, Cambridge, MA (US)

(21) Appl. No.: 15/622,261

(22) Filed: Jun. 14, 2017

Related U.S. Application Data
(63) Continuation of application No. PCT/US2015/065962, filed on Dec. 16, 2015.
Application No. PCT/US2015/065962 is a continuation of application No. PCT/US2015/065948, filed on Dec. 16, 2015.
(60) Provisional application No. 62/092,452, filed on Dec. 16, 2014.
(60) Provisional application No. 62/092,452, filed on Dec. 16, 2014.

(51) Int. Cl. C12Q 1/68

(52) U.S. Cl. CPC C12Q 1/6876

(57) ABSTRACT

Aspects of the present disclosure relate to systems, kits and methods that comprise a nucleic acid capture strand linked to a first dye molecule, a nucleic acid trigger strand longer than the capture strand and comprising (a) a capture domain that is complementary to the capture strand and (b) at least two concatenated domains, each of which comprises two subdomains, and a partially double-stranded nucleic acid comprising a single-stranded toehold domain having a nucleotide sequence complementary to one of the subdomains of the two subdomains of the concatenated domains, a double-stranded region linked to a second dye molecule and having a nucleotide sequence complementary to the other of the two subdomains of the concatenated domains, and a single-stranded hairpin loop having a nucleotide sequence that is complementary to the single-stranded toehold domain.

### RELATED APPLICATIONS

This application is a continuation of international application number PCT/US2015/065962 filed Dec. 16, 2015 and international application number PCT/US2015/065948 filed Dec. 16, 2015, both of which claim the benefit of U.S. Provisional Patent Application Ser. No. 62/092,452, filed Dec. 16, 2014, incorporated herein by reference.

### FEDERALLY SPONSORED RESEARCH

This invention was made with government support under Grant Nos. 1DP20D007292-01, 1R01EB018659-01 and 5R21HD072481-02 awarded by National Institutes of Health, Grant No. CCF-1317291 awarded by National Science Foundation, and Grant Nos. N00014-11-1-0914 and N00014-14-1-0610 awarded by Office of Naval Research. The government has certain rights in the invention.

### BACKGROUND

Fluorescence microscopy permits specific target detection at the level of single molecules and has become an invaluable tool in biological research. To transduce the biological information to a signal that can be imaged, a variety of fluorescent probes, such as organic dyes or fluorescent proteins with different colors, have been developed. Despite their success, the current probes have several limitations, including lack of programmability.

### SUMMARY

Provided herein are programmable deoxyribonucleic acid (DNA)-based fluorescent probes having tunable (e.g., digitally tunable) properties, such as, for example, tunable color and brightness. Methods of the present disclosure use structural nucleic acid (e.g., DNA) nanotechnology for producing sub-diffraction probes, referred to herein as “metafluorophores,” which can be triggered to assemble, in some embodiments, on a target molecule.

Thus, some aspects of the present disclosure provide systems (or kits) comprising a nucleic acid capture strand linked to a first dye molecule, a nucleic acid trigger strand longer than the capture strand and comprising (a) a capture domain that is complementary to the capture strand and (b) at least two concatenated domains, each of which comprises two subdomains, and a partially double-stranded nucleic acid comprising a single-stranded toehold domain having a nucleotide sequence complementary to one of the subdomains of the two subdomains of the concatenated domains, a double-stranded region linked to a second dye molecule and having a nucleotide sequence complementary to the other of the two subdomains of the concatenated domains, and a single-stranded hairpin loop having a nucleotide sequence that is complementary to the single-stranded toehold domain.

Some aspects of the present disclosure provide nucleic acid nanostructures (metafluorophores) comprising at least two photophysically-distinct subsets of dye molecules, wherein the distance between dye molecules of a single photophysically-distinct subset is greater than the distance at which the dye molecules self-quench, and the distance between any pair of dye molecules, one dye molecule from one photophysically-distinct subset and the other dye molecule from another photophysically-distinct subset, is at least the Förster resonance energy transfer (FRET) radius of the pair of dye molecules. The foregoing nucleic acid nanostructures are referred to herein as “metafluorophores.”Some aspects of the present disclosure provide pluralities of nucleic acid nanostructures (metafluorophores), each nanostructure comprising a unique set of dye molecules, wherein each set of dye molecules includes at least two photophysically-distinct subsets of dye molecules, wherein the distance between dye molecules of a single photophysically-distinct subset is greater than the distance at which the dye molecules self-quench, and the distance between any pair of dye molecules, one dye molecule from one photophysically-distinct subset and the other dye molecule from another photophysically-distinct subset, is at least the Förster resonance energy transfer (FRET) radius of the pair of dye molecules. It should be understood that in the context of a plurality of nucleic acid nanostructures, the phrase “each nanostructure” refers to each species of nanostructure (e.g., multiple nanostructures having the same barcode) and not necessarily a single nanostructure. For example, a plurality of nucleic acid nanostructure may contain two (or more) species of nanostructure, whereby one species has a first unique set of dye molecules (e.g., for identifying a first target) and the other species has a second unique set of dye molecules (e.g., for identifying a second target), wherein the first set is different from the second set (as each set is unique). A “unique” set of dye molecules refers to a combination of dye molecules (e.g., a combination of number and “color”) that is present only on a single nucleic acid nanostructure, or only on a single species of nucleic acid nanostructure. FIG. 3C shows an example of a plurality of nucleic acid nanostructures, each nanostructure comprising a unique set of dye molecules.

In some embodiments, the nucleic acid nanostructures have non-overlapping intensity distributions.

Some aspects of the present disclosure provide subset(s) of nucleic acid nanostructures of any one of the pluralities as provided herein, wherein each nanostructure of the subset contains at least three photophysically-distinct subsets of dye molecules, each photophysically-distinct subset of dye molecules has a different number of dye molecules, and the intensity distributions of nucleic acid nanostructures of the subset are non-overlapping.

In some embodiments, the distance between any pair of dye molecules of a single photophysically-distinct subset is at least 5 nm. For example, the distance between any pair of dye molecules of a single photophysically-distinct subset may be at least 10 nm. In some embodiments, the distance between any pair of dye molecules of a single photophysically-distinct subset is 5 nm to 100 nm (e.g., 5-90 nm, 5-80 nm, 5-70 nm, 5-60 nm, 5-50, 5-40 nm, 5-30 nm, 5-20 nm, 10-90 nm, 10-80 nm, 10-70 nm, or 10-60 nm). In some embodiments, the distance between any pair of dye molecules of a single photophysically-distinct subset may be 10 nm to 50 nm (e.g., 10-40 nm, 10-30 nm, or 10-20 nm). In some embodiments, the distance between any pair of dye molecules of a single photophysically-distinct subset may be 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm or 100 nm. In some embodiments, the distance between any pair of dye molecules of a single photophysically-distinct subset is no greater than the length, width or height of the nucleic acid nanostructure.

In some embodiments, the distance between any pair of dye molecules, one dye molecule from one photophysically-distinct subset and the other dye molecule from another photophysically-distinct subset, is at least 10 nm. For example, the distance between any pair of dye molecules, one dye molecule from one photophysically-distinct subset and the other dye molecule from another photophysically-distinct subset may be at least 15 nm. In some embodiments, the distance between any pair of dye molecules, one dye molecule from one photophysically-distinct subset and the other dye molecule from another photophysically-distinct subset, is 10 nm to 100 nm (e.g., 10-90 nm, 10-80 nm, 10-80 nm, 10-60 nm, 10-50 nm, 10-40 nm, 10-30 nm, or 10-20 nm). In some embodiments, the distance between any pair of dye molecules, one dye molecule from one photophysically-distinct subset and the other dye molecule from another photophysically-distinct subset may be 25 nm to 50 nm. In some embodiments, the distance between any pair of dye molecules, one dye molecule from one photophysically-distinct subset and the other dye molecule from another photophysically-distinct subset, is 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm or 100 nm.

In some embodiments, the nucleic acid nanostructure has a size of less than 200 nm. For example, the nucleic acid nanostructure may have a size of less than 150 nm.

In some embodiments, dye molecules of each photophysically-distinct subset are attached indirectly to a nucleic acid of the nanostructure.

In some embodiments, dye molecules of each photophysically-distinct subset are attached indirectly to a nucleic acid of the nanostructure through at least one single-stranded nucleic acid.

In some embodiments, the at least one single-stranded nucleic is 15 to 100 (e.g., 15-90, 15-80, 15-70, 15-60, 15-50, 15-40, 15-30, 20-200, 20-90, 20-80, 20-70, 20-60, 20-50, 20-40, 30-100, 30-90, 30-80, 30-70, 30-60 or 30-50) nucleotides in length.

In some embodiments, dye molecules of a single photophysically-distinct subset are grouped together within a defined region on the nanostructure.

In some embodiments, the nucleic acid nanostructures comprise at least three photophysically-distinct subsets of dye molecules. For example, the nucleic acid nanostructures may comprise three to ten (e.g., 3, 4, 5, 6, 7, 8, 9 or 10) photophysically-distinct subsets of dye molecules.

In some embodiments, the photophysically-distinct subsets of dye molecules are spectrally-distinct subsets of dye molecules.

In some embodiments, the photophysically-distinct subsets of dye molecules have different bleaching kinetics relative to each other. For example, one subset may bleach at a rate that is at least 10% (e.g., 15%, 20%, 25%, 30%, 35%, 40%, 45%, or 50%) faster than the rate at which another subset bleaches.

In some embodiments, the photophysically-distinct subsets of dye molecules have different photoswitchable properties relative to each other. In some embodiments, photophysically-distinct subsets of dye molecules behave differently under different buffer conditions, have different fluorescence lifetimes, and/or have different quantum yields.

Some aspects of the present disclosure provide nucleic acid nanostructures that comprise at least two spectrally-distinct subsets of dye molecules, wherein at least one subset comprises donor dye molecules (e.g., FIG. 19A, Cy3 and at least one subset comprises acceptor dye molecules (e.g., FIG. 19A, Alexa 647, and wherein the distance between any pair of donor and acceptor dye molecules is within the distance at which Förster resonance energy transfer (FRET) occurs between the pair. FRET pairs in close proximity show an intensity loss for the donor. However, if the acceptor bleaches over time, the donor intensity will increase, accordingly (FIG. 19A).

In some embodiments, the nanostructures comprise at least three spectrally-distinct subsets of dye molecules, wherein at least one subset comprises donor dye molecules and at least two subsets comprise acceptor dye molecules, and wherein the distance between any pair of donor and acceptor dye molecules is within the distance at which Förster resonance energy transfer (FRET) occurs between the pair.

In some embodiments, a donor dye molecule is proximal to at least two acceptor dye molecules such that the distance between the donor dye molecule and each acceptor dye molecule is within the distance at which FRET occurs between the donor dye molecule and each acceptor dye molecule.

In some embodiments, the at least two acceptor dye molecules are of the same subset. In some embodiments, the at least two acceptor dye molecules are of different subsets (e.g., each subset spectrally-distinct from the other).

Some aspects of the present disclosure provide nucleic acid nanostructures that comprise at least three photophysically-distinct subsets of dye molecules, wherein at least two of the photophysically-distinct subset of dye molecules are spectrally overlapping, and wherein the distance between any pair of dye molecules, one dye molecule from one spectrally-distinct subset and the other dye molecule from another spectrally-distinct subset, is within the distance where Förster resonance energy transfer (FRET) occurs.

In some embodiments, the donor dye of such a FRET pair has one acceptor dye of a spectrally distinct subset in its immediate vicinity (e.g., Alexa 647R1-Cy3G1 ̂ R2-G1)

In some embodiments, the donor dye of such a FRET pair has several acceptor dyes of one of the spectrally distinct subsets in its immediate vicinity (e.g., R1-G1-R1 ̂ R2-G1-R1).

In some embodiments the donor dye of such a FRET pair has several acceptor dyes of any of the spectrally distinct subsets in its immediate vicinity (e.g., R1-G1-R2).

Some aspects of the present disclosure provide nucleic acid nanostructures that comprise at least three photophysically-distinct subsets of dye molecules, wherein the distance between any pair of dye molecules, one dye molecule from one spectrally-distinct subset and the other dye molecule from another spectrally-distinct subset, is within the distance where Förster resonance energy transfer (FRET) occurs.

In some embodiments the donor dye of such a FRET pair has one acceptor dye of a photophysically-distinct subset in its immediate vicinity (e.g., R1-G1 ̂ R2-G1 ̂ R1-B1 ̂ R2-B1).

In some embodiments the donor dye of such a FRET pair has several acceptor dyes of one of the photophysically-distinct subsets in its immediate vicinity (e.g., R1-G1-R1̂ R2-G1-R2 ̂ R1-B1-R1 ̂ R2-B1R2).

In some embodiments the donor dye of such a FRET pair has several acceptor dyes of any of the photophysically-distinct subsets in its immediate vicinity (e.g., R1-G1-R2̂ R1-B1-R2).

Also provided herein are pluralities (e.g., at least two) nucleic acid nanostructures, each nanostructure of the plurality comprising a unique set of dye molecules.

In some embodiments, a nucleic acid nanostructure of the present disclosure is linked to a first single-stranded oligonucleotide that is complementary to a first region of a nucleic acid target (see, e.g., FIG. 22A). In some embodiments, the first single-stranded oligonucleotide is bound to (hybridized to) the first region of a nucleic acid target.

In some embodiments, the nucleic acid target comprises a second region complementary to and bound to a second single-stranded oligonucleotide, wherein the second single-stranded oligonucleotide is attached to a substrate. In some embodiments, the second single-stranded oligonucleotide is biotinylated. In some embodiments, the surface is coated in streptavidin and the second biotinylated single-stranded oligonucleotide is attached to the substrate via a biotin-streptavidin binding interaction. In some embodiments, the substrate is a glass or plastic substrate. Other means of attaching single-stranded oligonucleotides to a surface of a substrate are encompassed by the present disclosure (e.g., via other ligand-ligand binding interactions or via other linker molecules). In some embodiments, a first or second single-stranded oligonucleotide has a length of 10-50, 15-50, 20-30, 20-40, or 20-50 nucleotides, or is longer.

Provided herein are substrates comprising on a surface of the substrate a plurality of biotinylated single-stranded oligonucleotides, wherein at least some of the biotinylated single-stranded oligonucleotides are complementary to and bound to a region of a target nucleic acid, and wherein the first single-stranded oligonucleotide of a nucleic acid nanostructure is complementary to and bound to another region of the target nucleic acid (see, e.g., FIGS. 22A and 22B).

Also provided herein are methods of quantifying nucleic acid targets, comprising (a) applying target nucleic acids to a substrate comprising on a surface of the substrate a plurality of biotinylated single-stranded oligonucleotides, wherein the target nucleic acids comprise a first and second region, and wherein the biotinylated single-stranded oligonucleotides are complementary to the second region of the target nucleic acids; (b) applying to the substrate of (a) a plurality of nucleic acid nanostructures under conditions that result in binding of the nucleic acid nanostructures to nucleic acid targets; and (c) quantifying (e.g., imaging) nucleic acid nanostructures bound to nucleic acid targets.

### BRIEF DESCRIPTION OF DRAWINGS

FIGS. 1A-1G show examples of DNA-based metafluorophores of the present disclosure. FIG. 1A shows a schematic of an example of a labeling pattern for DNA origami-based metafluorophores. Cylinders represent DNA double helices. Selected strands are extended with 21 nucleotide (nt) “handles” on the 3′-end, which bind complementary fluorescently-labeled “anti-handles.” (“Handles” and “anti-handles” refer to complementary oligonucleotides (oligonucleotides that bind to each other).) Labeling patterns are represented as pictograms, where each colored dot represents a dye-labeled handle. FIGS. 1B-1D show that fluorescence intensities increase linearly with the number of dyes attached to a metafluorophore (e.g., 132 dyes per structure). Insets show diffraction-limited fluorescence images of metafluorophores and the corresponding labeling pattern (image sizes: 1.2×1.2 μm²). FIGS. 1E-1G show that metafluorophores allow dense labeling (e.g., ˜5 nm dye-to-dye distance) without self-quenching. Pictograms illustrate dense and sparse labeling patterns for 14 dyes. Corresponding intensity distributions of the two patterns overlap for each color, showing no significant change in intensity.

FIGS. 2A-2F show examples of multi-color metafluorophores. FIGS. 2A-2C show that “randomly” labeled metafluorophores may result in a significant decrease in fluorescence intensity (FIG. 2A, FIG. 2B) due to Förster resonance energy transfer (FRET), when labeled with spectrally distinct dyes. Metafluorophores with only 44 dyes of the same color serve as references (medium gray distributions). If Atto 647N, Cy3 and Atto 488 are all present on the same structure (44 dyes each), the intensity distributions (light gray) for Cy3 (FIG. 2B) and Atto 488 (FIG. 2C) are significantly shifted to lower values. However, this fluorophore arrangement does not provide an acceptor for Atto 647N fluorescence, thus its intensity distribution is not altered (FIG. 2A). Pictograms illustrate labeling patterns. FIGS. 2D-2F show that column-like metafluorophore labeling pattern prevents FRET. Metafluorophores labeled with 44 dyes of one species (medium gray) show intensity distributions identical to structures labeled with all three species. Pictograms illustrate labeling patterns.

FIGS. 3A-3G show examples of metafluorophores for intensity barcoding. FIG. 3A shows intensity distributions for Atto 488 (from left to right, 6 dye molecules per structure, 14 dye molecules per structure, 27 dye molecules per structure, 44 dye molecules per structure). Non-overlapping intensity distributions can be achieved by the precise control over the number of dye molecules per metafluorophore structure. FIG. 3B shows a fluorescence image of 124 distinct metafluorophores deposited on a glass surface (scale bar: 5 μm). FIG. 3C shows a matrix of representative fluorescence images of 124 distinct metafluorophores. FIG. 3D shows 124 metafluorophore-based intensity barcodes in one sample. A total of 5,139 barcodes were recorded, and all 124 barcode types were detected. FIG. 3E shows a subset of 25 out of 124 barcodes. 2,155 barcodes were recorded −86.5% were qualified barcodes, and 87.4% thereof were expected barcodes. FIG. 3F shows a subset of 12 out of 64 barcodes. All barcodes have all three fluorophore species, making their detection more robust. 521 barcodes were recorded −92.5% were qualified, and 95.4% thereof were expected barcodes. FIG. 3G shows a subset of 5 out of 20 barcodes. 664 barcodes were recorded −100% were qualified, and 99.6% thereof were expected barcodes.

FIGS. 4A-4C show triggered assembly of metafluorophores. FIG. 4A shows a schematic of triggered assembly of triangular metafluorophores constructed from ten metastable Cy3-labeled DNA hairpin strands. A nucleic acid “capture strand” (labeled with Alexa 647) is attached to a glass surface through biotin-streptavidin coupling. A longer “trigger strand” can hybridize to the capture strand. The trigger strand contains four concatenated domains ‘1-A,’ where the subdomain ‘1’ is 20 nucleotides in length, and subdomain ‘A’ is 12 nucleotides in length. Hairpin strands co-exist meta-stably in the absence of the trigger and only assemble into the desired structure upon exposure to the trigger. For example, the introduction of a repetitive single-stranded trigger initiates the assembly of kinetically trapped fluorescent hairpin monomers, which produce a second row of binding sites. These binding sites further enable the assembly of successive rows of monomers, with each row containing one fewer monomer than the previous. After assembly of 10 hairpins (labeled with Cy3) to a single trigger strand, no further trigger sequences are displayed and assembly is terminated, yielding a triangular-shaped metafluorophore of fixed dimensions. FIG. 4B shows fluorescence images of triangles assembled in situ on a glass surface. The capture strands are labeled with Alexa 647 and the hairpins with Cy3. DNA origami with 10 Cy3 and 44 Atto 488 dye molecules were added to the sample as intensity references. DNA origami structures can be identified at the positions where Atto 488 and Cy3 signals co-localize. In the schematic below the overlay fluorescence image, the one dark spot represents the Atto 488-labeled origami marker, and the lighter gray spots represent the expected overlay of Alexa 647-labeled capture strand and the triangle composed of Cy3-labeled hairpin monomers. The gray “x” symbols represent non-specific binding of hairpins to the surface. FIG. 4C shows that triangular metafluorophores (light gray) and reference DNA origami (dark gray)intensity distributions are overlapping, indicating the formation of the triangles.

FIG. 5 shows caDNAno DNA origami design. Circular DNA scaffold (light gray) is routed in horizontal loops to form 24 parallel helices. Staple strands (gray) connect parts of the scaffold and form the rectangle. Eight strands are biotinylated on the 5′-end (medium gray). Most 3′ and 5′-ends of the gray staple strands are on the same DNA origami face. However, biotin and dye functionalizations are intended to protrude on opposite faces. With the help of adjacent staples, the medium gray staples are shifted by one helix. This switches the 3′ and 5′-ends to the opposite face. Black crosses define base-skips, which are required to prevent the DNA origami from twisting.

FIGS. 6A-6K show schematics of examples of DNA origami staple layouts of single-color metafluorophores (6-132). Hexagons represent 3′-ends of all 176 staples, compare to FIG. 5. Dark gray shapes represent biotinylated staple strands, protruding on the opposite face. Black hexagons represent staples with 3′-handle extension (see Table 2). The pattern is the same for Atto 647N, Cy3 and Atto 488. FIG. 6A is a not a functionalized structure, corresponding to the caDNAno layout. FIG. 6B shows 6 dye molecules attached, FIG. 6C shows 12 dye molecules attached, FIG. 6D shows 18 dye molecules attached, FIG. 6E shows 24 dye molecules attached, FIG. 6F shows 30 dye molecules attached, FIG. 6G shows 54 dye molecules attached, FIG. 6H shows 72 dye molecules attached, FIG. 61 shows 84 dye molecules attached, FIG. 6J shows 108 dye molecules attached, and FIG. 6K shows132 dye molecules attached.

FIGS. 7A-7C show the linear dependence of intensity with number of dyes per DNA origami structure (calibrated). From 6 to 132 dyes per DNA origami, the intensity scales linearly for Atto 647N (FIG. 7A), Cy3 (FIG. 7B) and Atto 488 (FIG. 7C). Investigated samples are identical to those in FIG. 1. However, samples contained the structure of interest and additionally a second DNA origami with a significantly different dye count as reference. This allows comparison and calibration of measured intensities and thereby reduces sample-to-sample variations. Corresponding data in FIG. 1 is not calibrated.

FIGS. 8A-8C show intensity distributions for 6 to 132 dye molecules. Data corresponds to FIG. 7, where mean and standard deviation of the distributions are plotted. FIG. 8A shows Atto 647N. FIG. 8B shows Cy3. FIG. 8C shows Atto 488. Investigated samples contained the structure of interest and a second DNA origami with a significantly different dye molecule count as reference. Reference intensity distributions are not shown.

FIGS. 9A-9C show excitation power variation data. All DNA origami-based metafluorophore recordings were measured using a Zeiss Colibri LED light source. The measured intensity of a 30 dye metafluorophore scales linear with the applied excitation intensity for Atto 647N (FIG. 9A), Cy3 (FIG. 9B) and Atto 488 (FIG. 9C). More than 12,000 metafluorophores were evaluated per data point. Camera integration times were constant at 10 seconds. All subsequent measurements throughout this study were performed at 60%.

FIGS. 10A-10C show integration time variation data. All DNA origami-based metafluorophore recordings were measured using a Hamamatsu ORCA Flash 4.0 sCMOS camera. Integration times were varied from 2 s to 10 s per recording and show a linear increase in intensity of a 30 dye metafluorophore for Atto 647N (FIG. 10A), Cy3 (FIG. 10B) and Atto 488 (FIG. 10C) at 60% excitation intensity. More than 12,000 metafluorophores were evaluated per data point. All subsequent measurements throughout this study were performed at 10 s integration time.

FIGS. 11A-11C show refocusing performance data. While repeated focusing attempts may lead to imaging in different focal planes, different focal planes may yield different intensities of a single target. The same samples, containing DNA origami based metafluorophores with 30 dyes, were imaged and refocused five times for Atto 647N (FIG. 11A), Cy3 (FIG. 11B) and Atto 488 (FIG. 11C). Plots are normalized to the average value (colored line).

FIGS. 12A-12C show photostability data. Repeated recording of the same area causes photobleaching of the dyes. The measured intensity drops exponentially. Measurements were performed at 60% excitation power and integration times of 10 s per frame on a 30 dye DNA origami metafluorophore for Atto 647N (−0.77%, FIG. 12A), Cy3 (−1.37%, FIG. 12B) and Atto 488 (−2.80% per acquisition, FIG. 12C).

FIGS. 13A-13F show schematics of examples of DNA origami staple layouts used in a self-quenching study. FIGS. 13A-13C show sparse dye patterning on DNA origami with ˜15 nm dye-to-dye distance, for Atto 647N (FIG. 13A), Cy3 (FIG. 13B) and Atto 488 (FIG. 13C). FIGS. 13D-13F show dense dye patterning on DNA origami with ˜5 nm dye-to-dye distance, for Atto 647N (FIG. 13D), Cy3 (FIG. 13E) and Atto 488 (FIG. 13F).

FIGS. 14A-14H show an example of FRET investigation dye patterning (random and column-wise). FIGS. 14A-14D show mixed dye patterns, corresponding to FIGS. 2A-2C. FIGS. 14E-14H show column-wise dye pattern with inter-color spacing >10 nm, corresponding to FIG. 2D-2F.

FIGS. 15A-15D show examples of intensity barcode dye patterns. The column-wise dye pattern separates distinct dyes>10 nm and, thus, prevents FRET. FIG. 15A shows 6, FIG. 15B shows 14, FIG. 15C shows 27 and FIG. 15D shows 44 dyes attached per color. These layouts were used to independently control brightness levels for all three colors in the barcode studies.

FIGS. 16A-16C show intensity distributions of a 25/124 barcode study. Exemplary intensity distributions of 25 distinct metafluorophores combined in one sample for Atto 647N (FIG. 16A), Cy3 (FIG. 16B) and Atto 488 (FIG. 16C). Four levels (corresponding to 6, 14, 27 and 44 dye molecules) are clearly distinguishable. Overlapping regions in between peaks were identified (see Methods and Materials) and barcode displaying corresponding intensities were classified as unqualified.

FIG. 17 shows a triggered-assembly formation gel assay. See Methods and Materials for details. Capture strands (CAP) are labeled with Alexa 647 (lane 1, reference), hairpins (HP) with Cy3 (lane 3, reference). Trigger strands (T) are unlabeled. Lane 1 (1 pmol CAP) and 3 (12 pmol) serve as reference for CAP and HP migration speeds. Lanes 4-7 show reactions performed at 30° C. and lanes 8-11 at 24° C., respectively (1 pmol CAP each). Control lanes 7 and 11 are missing the (T) strand, thereby inhibiting triangle formation. Lanes only show CAP and HP bands, in agreement with the reference bands. Assembly reactions in lanes 5 and 9 had 12 fold excess of HP strands over CAP strands (10.9 over T), and triangles (10 HP per triangle) are formed as indicated by the strong band migrating slower than the reference bands. The presence of a CAP reference band indicates that not all CAP strands formed a triangle. Since HP strands are in slight stoichiometric excess in regards to the triangles, a weak HP band is notable. Lanes 6 and 10 contain reactions with higher HP excess. Product bands appear to migrate slightly slower than the product bands in lane 5 and 9, indicating only marginally increased triangle size. Reactions in lanes 4 and 8 had insufficient HP to fully assemble a triangle (<5 of 10 strands). Lanes show a faster product band than the corresponding 12× and 20× lanes, implying only partly assembled triangles. The Cy3 HP band is very weak, indicating complete usage of HP strands.

FIG. 18A shows that several intensity levels can be achieved by varying the amount of fluorophores on a DNA nanostructure. FIG. 18B shows combinatorial labeling of nanostructures with spectrally-distinct dyes and different intensity levels. Each zone in the nanostructure may be equipped with different amounts of fluorophores and, therefore, have a different intensity level. FIG. 18C shows that different fluorophores of the same color show different dye stability and can be identified by their bleaching signature. FIG. 18D shows combinatorial labeling of nanostructures with spectrally-distinct dyes and different dye stability. The combinatorial possibilities are increased.

FIG. 19A shows that FRET pairs in close proximity will show an intensity loss for the donor. If the acceptor bleaches over time, the donor intensity will increase accordingly. Depending on the amount of FRET pairs the intensity signature will vary. FIG. 19B shows that usage of multiple colors will increase the combinatorial possibilities. FIG. 19C shows that with alternation of the mean acceptor neighbors to a FRET donor it is possible to “delay” the FRET increase.

FIG. 20A shows two barcodes specifically dimerized by the presence of a DNA/RNA target. The barcodes carry handles complementary to parts of the target. FIG. 20B shows that a target may open a DNA hairpin which in turn enables dimerization. FIG. 20C shows that one barcode may be sufficient, and a second component is solely required to report dimerization. FIG. 20D shows that the auxiliary strand may be part of one of the monomers.

FIG. 21A shows time-lapsed fluorescence micrographs of a sample comprised of two spectrally indistinct metafluorophore species: one containing 44 Atto 647N dyes (more photostable) and one containing 44 Alexa647 dyes (less photostable). Images were acquired at t₁=0 s, t₂=20 s, and t₃=40 s with an integration time of 10 s, while the sample was constantly illuminated during acquisition (i.e. the total illumination time was 60 s). The time-lapsed micrographs show two species where one bleaches faster than the other. The two species can be visually identified by superimposing the images taken at t₁ and t₃. The metafluorophore containing more photostable dyes (e.g., Atto 647N) appears light, while the one with the less photostable dyes (i.e. Alexa647) appears dark gray. Scale bar: 5 μm. The fluorescence decay constant can be used as a parameter to quantitatively describe the photostability. The decay constant is obtained by fitting a single exponential decay to the intensity vs. time trace. FIG. 21B shows intensity vs. decay constant histograms for three different metafluorophore samples containing Atto647N dyes (left), Alexa647 dyes (right), and both dyes (center), respectively (Note that only one species was present in each sample). FIG. 21C illustrates a one-dimensional histogram of the decay constants, showing three distinguishable decay constant distributions (schematics in the legend show the dye arrangement on the metafluorophores).

FIGS. 22A-22C show an example of quantitative nucleic acid detection. FIGS. 22A and 22B show schematics of a hybridization reaction. A metafluorophore is programmed to hybridize to a region (t1) of a specific nucleic acid target. A biotinylated capture strand binds to a second region (a) of the specific nucleic acid target and is thus capable of immobilizing the triplet (capture strand, nucleic acid target and metafluorophore) on a streptavidin coated surface. Each positively identified metafluorophore indicates a single nucleic acid target. FIG. 22C is a bar graph showing that the number of detected targets is directly proportional to their concentration in the sample of interest. Targets were added at with defined concentrations (dark gray bars) and subsequently identified with in the expected ratios (light gray bars). The lowest target concentration (targets 3 and 4) was 1.5 pM. Sequences left to right, top to bottom: SEQ ID NO: 197-199.

### DETAILED DESCRIPTION

Fluorescence microscopy permits imaging molecules in bulk. It is highly specific, highly sensitive, and it permits the detection of single biomolecules. This is usually achieved with fluorescent tags such as genetically-encodable fluorescent proteins, organic dyes, or inorganic fluorescent nanoparticles. While fluorescent proteins can be co-expressed with the target protein of interest, organic and inorganic dyes must be coupled, for example, to antibodies, small molecules or DNA, in order to specifically label targets, such as proteins or nucleic acids.

A major advantage of fluorescence microscopy is the possibility of simultaneously detecting and identifying multiple distinct molecular species in one sample by using spectrally distinct fluorescent tags (colors), referred to as multiplexing. Nonetheless, this multiplexed detection is restricted by the number of unambiguously detectable spectral colors in the visible range. The rather broad emission spectra of organic fluorophores limits spectral multiplexing to about 4-5 distinct dyes.

Thus, fluorescence microscopy is in need of a novel type of programmable tag, which permits the unambiguous detection of ideally hundreds of distinct target species, while maintaining desired properties of “classical” dyes such as their nanoscale size and target labeling capabilities. However, only limited success towards programmable tags has been achieved, mainly due to the lack of independent and precise control of properties such as intensity, color, size and molecular recognition.

The present disclosure provides a general framework for engineering sub-diffraction-sized tags having digitally-tunable brightness and color using tools from structural DNA nanotechnology. Each tag is composed of multiple detectable labels organized in a spatially-controlled fashion in a compact sub-diffraction volume. This renders the tags indistinguishable from traditional organic fluorophores when using a diffraction-limited microscope. Thus, the tag of the present disclosure is referred to as a “metafluorophore.” Examples of detectable labels for use as provided herein include, without limitation, inorganic and organic fluorophores, fluorescent proteins, fluorescent nanoparticles, inorganic nanoparticles, nanodiamonds and quantum dots.

Unlike a traditional fluorophore, a metafluorophore has digitally and independently tunable optical properties, such as programmable intensity levels and color mixing ratios. To produce these metafluorophores, nucleic acid (e.g., DNA) nanostructures were used as a platform to organize organic fluorophores in a sub-diffraction volume with precisely prescribed copy number, color ratio, and spatial control. The independent programmability of both intensity and color enables the construction of over one hundred explicitly programmed metafluorophores that can serve as nanoscale intensity barcodes for high content imaging.

There are several ways to create unique barcode signatures based on properties such as geometry and intensity. Geometrical barcoding may be achieved by spacing distinct fluorescent sites beyond the spatial resolution of the used imaging system (e.g., greater than 250 nm for diffraction-limited and greater than 20-40 nm for super-resolution systems). In combination with spectrally-distinct fluorophores, combinatorial labeling exponentially increases the number of possible barcodes. Nonetheless, geometrical barcoding leads to an increased label size due to the necessity of spacing fluorophores sufficiently apart for accurate detection. None of the existing sub-micrometer barcode systems based on geometry or fluorescence intensity provides, for example, hundreds of barcodes with sizes below 100-200 nm, which is advantageous for in situ labeling.

In intensity barcoding implementations, distinguishable barcodes may be produced by controlling the number of fluorophores per species, thus allowing the unambiguous detection of different intensity levels. Compared to geometrical barcodes, an advantage of intensity barcodes is that they require neither the construction nor the detection of spatially resolvable fluorescent features. Thus, intensity barcodes can be much smaller.

Existing intensity barcodes are bulky, micron-sized structures. This large spatial size ensures robust separation between intensity levels because these barcodes lack the molecular programmability of fluorophore number, spacing and positioning, leading to unwanted photophysical effects such as self-quenching and Förster Resonance Energy Transfer (FRET) between dye molecules. The metafluorophores of the present disclosure, by contrast, in some embodiments, feature precise molecular control over number, spacing and arrangement of fluorophores in a nanoscale volume and, thus, are ideally poised to serve as a platform for intensity barcodes without the discussed drawbacks.

### Nucleic Acid Nanostructures

Embodiments of the present disclosure provide nucleic acid nanostructures that comprise a particular species, number and/or arrangement of dye molecules. A “nucleic acid nanostructure,” as used herein, refers to nucleic acids that form (e.g., self-assemble) two-dimensional (2D) or three-dimensional (3D) shapes (e.g., reviewed in W. M. Shih, C. Lin, Curr. Opin. Struct. Biol. 20, 276 (2010), incorporated by reference herein). Nanostructures may be formed using any nucleic acid folding or hybridization methodology. One such methodology is DNA origami (see, e.g., Rothmund, P. W. K. Nature 440 (7082): 297-302 (2006), incorporated by reference herein). In a DNA origami approach, a nanostructure is produced by the folding of a longer “scaffold” nucleic acid strand through its hybridization to a plurality of shorter “staple” oligonucleotides, each of which hybridize to two or more non-contiguous regions within the scaffold strand. In some embodiments, a scaffold strand is at least 100 nucleotides in length. In some embodiments, a scaffold strand is at least 500, at least 1000, at least 2000, at least 3000, at least 4000, at least 5000, at least 6000, at least 7000, or at least 8000 nucleotides in length. The scaffold strand may be naturally or non-naturally occurring. Staple strands are typically less than 100 nucleotides in length; however, they may be longer or shorter depending on the application and depending upon the length of the scaffold strand. In some embodiments, a staple strand may be 15 to 100 nucleotides in length. In some embodiments, a staple strand is 25 to 50 nucleotides in length.

In some embodiments, a nucleic acid nanostructure may be assembled in the absence of a scaffold strand (e.g., a scaffold-free structure). For example, a number of oligonucleotides (e.g., less than 200 nucleotides or less than 100 nucleotides in length) may be assembled to form a nucleic acid nanostructure.

Other methods for assembling nucleic acid nanostructures are known in the art, any one of which may be used herein. Such methods are described by, for example, Bellot G. et al., Nature Methods, 8: 192-194 (2011); Liedl T. et al, Nature Nanotechnology, 5: 520-524 (2010); Shih W. M. et al, Curr. Opin. Struct. Biol., 20: 276-282 (2010); Ke Y. et al, J. Am. Chem. Soc, 131: 15903-08 (2009); Dietz H. et al, Science, 325: 725-30 (2009); Hogberg B. et al, J. Am. Chem. Soc, 131: 9154-55 (2009); Douglas S. M. et al, Nature, 459: 414-418 (2009); Jungmann R. et al, J. Am. Chem. Soc, 130: 10062-63 (2008); Shih W. M., Nature Materials, 7: 98-100 (2008); and Shih W. M., Nature, A11: 618-21 (2004), each of which is incorporated herein by reference in its entirety.

A nucleic acid nanostructure may be assembled into one of many defined and predetermined shapes including without limitation a hemi-sphere, a cube, a cuboidal, a tetrahedron, a cylinder, a cone, an octahedron, a prism, a sphere, a pyramid, a dodecahedron, a tube, an irregular shape, and an abstract shape. The nanostructure may have a void volume (e.g., it may be partially or wholly hollow). In some embodiments, the void volume may be at least 25%, at least 50%, at least 75%, at least 85%, at least 90%, or more of the volume of the nanostructure. Thus, in some embodiments, nucleic acid nanostructures do not comprise a solid core. In some embodiments, nucleic acid nanostructures are not circular or near circular in shape. In some embodiments, nucleic acid nanostructures are not a solid core sphere. Depending on the intended use, nucleic acid nanostructures may be assembled into a shape as simple as a two-dimensional sheet or as complex as a three-dimensional lattice (or even more complex).

Nucleic acid nanostructures may be made of, or comprise, DNA, RNA, modified DNA, modified RNA or a combination thereof.

In some embodiments, nucleic acid nanostructures are rationally designed. A nucleic acid nanostructure is herein considered to be “rationally designed” if nucleic acids that form the nanostructure are selected based on pre-determined, predictable nucleotide base pairing interactions that direct nucleic acid hybridization. For example, nucleic acid nanostructures may be designed prior to their synthesis, and their size, shape, complexity and modification may be prescribed and controlled using certain select nucleotides (e.g., oligonucleotides) in the synthesis process. The location of each nucleic acid in the structure may be known and provided for before synthesizing a nanostructure of a particular shape. The fundamental principle for designing, for example, self-assembled nucleic acid nanostructures is that sequence complementarity in nucleic acid strands is selected such that, by pairing up complementary segments, the nucleic acid strands self-organize into a predefined nanostructure under appropriate physical conditions. Thus, in some embodiments, nucleic acid nanostructures are self-assembling.

Examples of nucleic acid nanostructures for use in accordance with the present disclosure include, without limitation, lattices (E. Winfree, et al. Nature 394, 539 (1998); H. Yan, et al. Science 301, 1882 (2003); H. Yan, et al. Proc. Natl. Acad. of Sci. USA 100, 8103 (2003); D. Liu, et al. J. Am. Chem. Soc. 126, 2324 (2004); P. W. K. Rothemund, et al. PLoS Biology 2, 2041 (2004)), ribbons (S. H. Park, et al. Nano Lett. 5, 729 (2005); P. Yin, et al. Science 321, 824 (2008)), tubes (H. Yan Science (2003); P. Yin (2008)), finite two-dimensional (2D) and three dimensional (3D) objects with defined shapes (J. Chen, N. C. Seeman, Nature 350, 631 (1991); P. W. K. Rothemund, Nature 440, 297 (2006); Y. He, et al. Nature 452, 198 (2008); Y. Ke, et al. Nano. Lett. 9, 2445 (2009); S. M. Douglas, et al. Nature 459, 414 (2009); H. Dietz, et al. Science 325, 725 (2009); E. S. Andersen, et al. Nature 459, 73 (2009); T. Liedl, et al. Nature Nanotech. 5, 520 (2010); D. Han, et al. Science 332, 342 (2011)), macroscopic crystals (J. P. Meng, et al. Nature 461, 74 (2009)), single-stranded tiles (SSTs) (see, e.g., Wei B. et al. Nature 485: 626, 2012 and International Publication Number WO 2014/074597, published 15 May 2014, each incorporated by reference herein), and structures assembled from nucleic acid “bricks” (see, e.g., Ke Y. et al. Science 388:1177, 2012; International Publication Number WO 2014/018675 A1, published 30 Jan. 2014, each incorporated by reference herein). Other nucleic acid nanostructures may be used as provided herein.

In some embodiments, a nucleic acid nanostructure of the present disclosure has a size (e.g., diameter, length, width and/or height) of 200 nm or less. For example, a nucleic acid nanostructure may have a size of less than 200 nm, less than 175 nm, less than 150 nm, less than 125 nm, less than 100 nm or less than 50 nm. In some embodiments, a nucleic acid nanostructure may have a size 100 nm or less.

### Dye Molecules

Nucleic acid nanostructures of the present disclosure, in some embodiments, comprise at least two photophysically-distinct subsets of dye molecules. A “dye molecule” refers to a molecule that exhibits one or more photophysical processes. A dye molecule, or a subset of dye molecules, is considered “photophysically-distinct” if it can be distinguished from other dye molecules based on one or more photophysical processes exhibited by the dye molecule or subset of dye molecules. Examples of photophysical processes include, without limitation, energy transfer and electron (or charge) transfer. Specific properties that are based on energy transfer and/or electron transfer include, for example, spectral properties, photostability, photoswitchable properties, blinking kinetics, response on buffer exchange, fluorescence lifetime and quantum yield.

In some embodiments, dye molecules are “spectrally distinct.” Spectrally distinct dye molecules may have a different emission spectrum but the same excitation spectrum relative to one another, or the same emission spectrum but with different excitation spectrum relative to one another. Differences in emission and/or excitation spectra can be detected using, for example, instrumentation (e.g., hardware or software) that relies on filtering or ‘linear unmixing’ algorithmns (see, e.g., Averbuch et al. Remote Sens. 2012, 4, 532-560). For example, Atto 647N, Atto655, Cy5 and Alexa 647 (red) are spectrally distinct from Atto 565, Cy3 and Cy3b (green), which are spectrally distinct from Atto488 and Alexa488 (blue). By comparison, Atto 647N, Atto655, Cy5 and Alexa 647 (red) are spectrally overlapping dye molecules. Similarly, Atto 565, Cy3 and Cy3b (green) are spectrally overlapping dye molecules, and Atto488 and Alexa488 (blue) are spectrally overlapping dye molecules.

In some embodiments, dye molecules are distinguished based on photostability. For example, different dye molecules may have different bleaching kinetics. “Bleaching kinetics” refers to the kinetics (e.g., rate) of a reaction in which a dye molecule is bleached, or loses the ability to fluoresce. In some embodiments, dye molecules are spectrally overlapping but have different bleaching kinetics. For example, Atto647N and Alexa 647 are spectrally overlapping but have different bleaching kinetics.

In some embodiments, dye molecules are distinguished based on photoswitchable properties. A “photoswitchable” dye molecules refers to a molecule with fluorescence that, upon excitation at a certain wavelength, can be switched on or off by light in a reversible manner. Phostoswitchable properties may be impacted by, for example, the chemical environment of the molecule (e.g., molecules in buffer without or without salt, thiols and/or enzymes).

A “photophysically-distinct subset” of dye molecules refers to a subset of the same dye molecules (e.g., a group of Atto 647N dye molecules, a subset of Cy3 dye molecules, or a group of Atto 488 dye molecules) that is distinguished from other subsets of dye molecules based on the photophysical properties of the dye molecules of the subset. For example, a subset of “red” Atto 647N dye molecules is considered to be photophysically-distinct from (and more specifically, spectrally-distinct from) a subset of “green” Cy3 dye molecules Likewise, a subset of “red” Atto 647N dye molecules is photophysically-distinct from a subset of “blue” Atto 488 dye molecules, and a subset of “blue” Atto 488 dye molecules is photophysically-distinct from a subset of “green” Cy3 dye molecules.

In some embodiments, the distance between dye molecules of a photophysically-distinct subset is greater than the distance at which the dye molecules self-quench. Quenching refers to a process that decreases the fluorescence intensity of a dye molecule. Dye molecules of a pair (e.g., two dye molecules of the same species), for example, are considered to “self-quench” when their proximity to each other is such that their fluorescent intensity decreases by at least 5% relative to the fluorescent intensity of an isolated dye molecule of the pair. This may occur through contact quenching or FRET. In some embodiments, dye molecules are considered to self-quench when their proximity to each other is such that their fluorescent intensity decreases by at least 5% to 100%. For example, dye molecules are considered to self-quench when their proximity to each other is such that their fluorescent intensity decreases by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

The distance at which dye molecules (e.g., fluorescent molecules) self-quench depends, in part, on the species of the dye molecule (e.g., Atto 647N, Cy3, Atto 488), including its photophysical properties. In some embodiments, the distance at which dye molecules (e.g., fluorescent molecules) self-quench ranges from contact (e.g., 0.1 nm to 50 nm), or more, measured from the approximate center of the dye molecule. In some embodiments, the distance at which dye molecules (e.g., fluorescent molecules) self-quench is at least 5 nm, at least 10 nm or at least 15 nm. In some embodiments, the distance at which dye molecules (e.g., fluorescent molecules) self-quench may be less than 5 nm (e.g., 4 m, 3 nm, 2 nm or 1 nm). In some embodiments, the distance at which dye molecules (e.g., fluorescent molecules) self-quench is 5 nm to 50 nm. For example, the distance at which dye molecules (e.g., fluorescent molecules) self-quench may be 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm or 50 nm. In some embodiments, the distance at which dye molecules (e.g., fluorescent molecules) self-quench may be 5 nm to 100 nm, 5 nm to 75 nm, 5 nm to 50 nm, 5 nm to 25 nm, 5 nm to 15 nm, or 5 nm to 10 nm.

In some embodiments, the distance between any pair of dye molecules, one dye molecule from one photophysically-distinct subset (e.g., a subset of Atto 647N dye molecules) and the other dye molecule from another photophysically-distinct subset (e.g., a subset of Cy3 dye molecules), is at least the Förster resonance energy transfer (FRET) radius of the pair of dye molecules. FRET is a mechanism describing energy transfer between two light-sensitive molecules. A donor dye molecule, initially in its electronic excited state, may transfer energy to an acceptor dye molecule through non-radiative dipole-dipole coupling. The efficiency of this energy transfer is inversely proportional to the sixth power of the distance between donor and acceptor, making FRET sensitive to small changes in distance. Measurements of FRET efficiency can be used to determine if two dye molecules are within a certain distance of each other. The “FRET radius” of a pair of dye molecules refers to the distance at which the energy transfer efficiency is 50%.

The FRET radius of a pair of dye molecules (e.g., fluorescent molecules) depends, in part, on the species of the dye molecule (e.g., Atto 647N, Cy3, Atto 488), including its photophysical properties. In some embodiments, the FRET radius of a pair of dye molecules (e.g., fluorescent molecules) is 1 nm to 100 nm, or more. For example, the FRET radius of a pair of dye molecules (e.g., fluorescent molecules) may be 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 55 nm, 60 nm, 65 nm, 70 nm, 75 nm, 80 nm, 85 nm, 90 nm, 95 nm or 100 nm. In some embodiments, the FRET radius of a pair of dye molecules (e.g., fluorescent molecules) may be 1 nm to 100 nm, 1 nm to 75 nm, 1 nm to 50 nm, 1 nm to 25 nm, 1 nm to 15 nm, 10 nm to 100 nm, 10 nm to 75 nm, 10 nm to 50 nm, 10 nm to 25 nm, or 10 nm to 15 nm. In some embodiments, the FRET radius of a pair of dye molecules (e.g., fluorescent molecules) may be at least 5 nm, at least 10 nm, at least 15 nm or at least 20 nm. In some embodiments, the FRET radius of a pair of dye molecules (e.g., fluorescent molecules) may be less than 10 nm (e.g., 9 m, 8 nm, 7 nm, 6 nm or 5 nm).

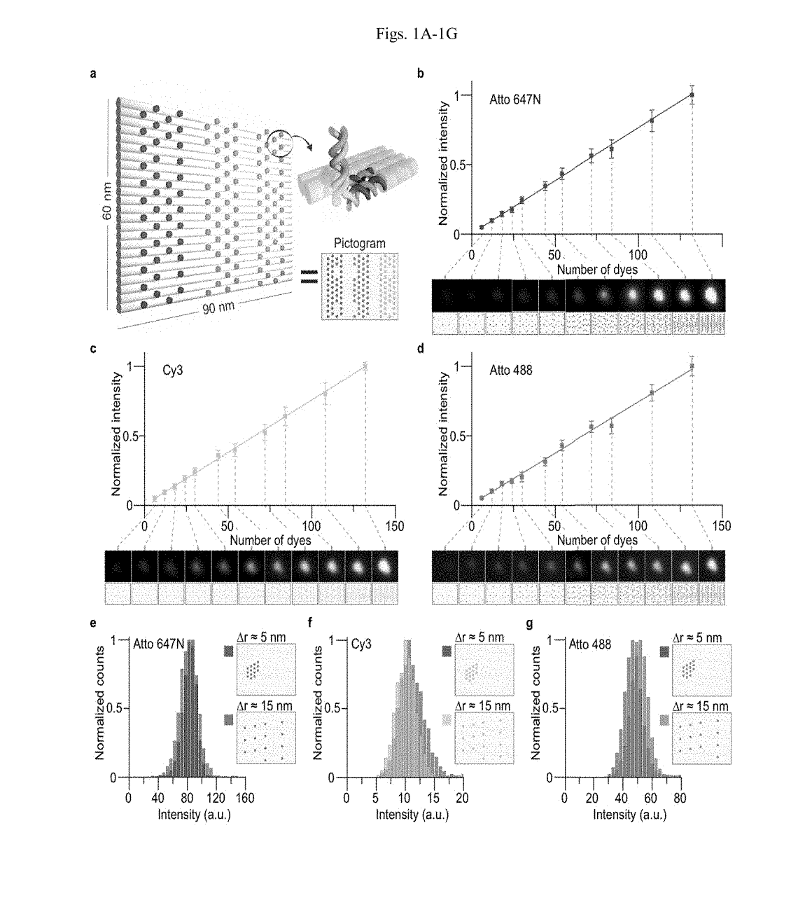

Dye molecules of a photophysically-distinct subset may be a homogenous subset grouped together within a defined region on the nanostructure. For example, FIG. 1A shows three photophysically-distinct (e.g., spectrally-distinct) subsets of dye molecules: a subset containing a “red” species, a subset containing a “blue” species, and a subset containing a “green” species. Each of the three photophysically-distinct subsets contain a homogeneous (e.g., the same) population of dye molecules. The distance between dye molecules of the photophysically-distinct “red” subset and dye molecules of the photophysically-distinct “blue” subset is at least the FRET radius of any pair of dye molecules, one molecule from the “red” subset and one molecule from the “blue” subset. Likewise, the distance between dye molecules of the photophysically-distinct “blue” subset and dye molecules of the photophysically-distinct “green” subset is at least the FRET radius of any pair of dye molecules, one molecule from the “blue subset and one molecule from the “green” subset.

In some embodiments, dye molecules of a photophysically-distinct subset may be intermingled with dye molecules of another photophysically-distinct subset as long as the distance between any pair of dye molecules, one dye molecule from one photophysically-distinct subset (e.g., “red”) and the other dye molecule from another photophysically-distinct subset (e.g., “blue”), is at least the FRET radius of the pair. Thus, in some embodiments, a nucleic acid nanostructure comprises a region containing a set a mixed population of photophysically-distinct dye molecules that do not exhibit self-quenching or FRET processes.

In some embodiments, a dye molecule is attached indirectly to a nucleic acid nanostructure (that is, a nanostructure is indirectly “labeled” with a dye molecule). For example, a dye molecule may be attached indirectly to a nucleic acid nanostructure via a “handle” and “anti-handle” (Rothemund, Nature 440, 297-302 (2006), incorporated by reference herein). At the position where a dye molecule is intended to be attached, a nucleic acid of the nanostructure may be extended with a short single-stranded nucleic acid, referred to as a “handle.” In some embodiments, the length of a handle is 10 nucleotides (nt) to 100 nt. For example, the length of a handle may be 10 to 90 nt, 10 to 80 nt, 10 to 70 nt, 10 to 60 nt, 10 to 50 nt, 10 to 40 nt, 10 to 30 nt, 15 to 100 nt, 15 to 90 nt, 15 to 80 nt, 15 to 70 nt, 15 to 60 nt, 15 to 50 nt, 15 to 40 nt, or 15 to 30 nucleotides. In some embodiments, the length of a handle may be 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt or 30 nucleotides. A complementary single-stranded nucleic acid, referred to as an “anti-handle,” is functionalized with the dye molecule intended to be attached to the nanostructure. In some embodiments, the dye molecule is covalently attached to the anti-handle. In some embodiments, the dye molecule is non-covalently attached to the anti-handle. Anti-handles are designed to be complementary to and to hybridize specifically to handles on a nanostructure. A handle/anti-handle imparts programmability to the dye molecules. For example, with reference to FIG. 1A, using orthogonal handle and anti-handle sequences, the three differently colored (e.g., red, blue and greed) molecules were “programmed” to attach to the nanostructure as homogenous groups of molecules. A handle and/or anti-handle may be, for example, a DNA or RNA handle and/or anti-handle.

In some embodiments, labeling of a nucleic acid nanostructure with a dye molecule can be achieved either by direct hybridization to a DNA or RNA strand on a nanostructure (e.g., handle/anti-handle-binding), or mediated by using antibodies or small molecule binders for protein labeling (see, e.g., Liu, Y., et al. Angew Chem Int Ed Engl 44, 4333-4338 (2005); Rinker, S., et al. Nat Nanotechnol 3, 418-422 (2008), incorporated by reference herein).

In some embodiments, a nucleic acid nanostructure is labeled directly with a dye molecules. For example, a dye molecule may be covalently or non-covalently attached to a nucleic acid strand of the nanostructure. In some embodiments, more than one dye molecule may be covalently or non-covalently attached to a nucleic acid strand of the nanostructure. For example, a nucleic acid strand may contain a dye molecule at its 3′ end, its 5′ end and/or it can be labeled internally (any region between the 3′ and 5′ ends).

A nanostructure of the present disclosure may comprise photophysically-distinct subsets of dye molecules that are each distinguished based on one or more photophysical processes. In some embodiments, a nucleic acid nanostructure comprises at least two photophysically-distinct subsets of dye molecules. For example, a nucleic acid nanostructure may comprise at least 3, at least 4, at least 5, at least 6, at least 7, at least 8, at least 9, or at least 10, or more photophysically-distinct subsets of dye molecules. In some embodiments, a nucleic acid nanostructure comprises 2 to 10, 3 to 10, 4 to 10 or 5 to 10 photophysically-distinct subsets of dye molecules. The photophysically-distinct subsets of dye molecules may be spectrally-distinct, have distinct bleaching kinetics, have distinct photoswitchable properties, or a combination of any two or three of the foregoing, for example.

The number of dye molecules within a photophysically-distinct subset of dye molecules may vary, depending on the desired intensity of the subset. In some embodiments, a photophysically-distinct subset of dye molecules contains 5 to 100 dye molecules. For example, a photophysically-distinct subset of dye molecules may contain 5, 6, 7, 8, 9, 10, 11, 12, 13, 14, 15, 16, 17, 18, 19, 20, 21, 22, 23, 24, 25, 26, 27, 28, 28, 30, 31, 32, 33, 34, 35, 36, 37, 38, 39, 40, 41, 42, 43, 44, 45, 46, 47, 48, 49 or 50 dye molecules. In some embodiments, a photophysically-distinct subset of dye molecules may contain 50, 55, 60, 65, 70, 75, 80, 85, 90, 95, 100 or more dye molecules.

A nucleic acid nanostructure the present disclosure typically has at least two photophysically-distinct subsets of dye molecules, each containing the same or different number of dye molecules. For example, a nanostructure may contain photophysically-distinct subset X, a photophysically-distinct subset Y, and a photophysically-distinct subset Z, wherein subset X contains n dye molecules, subset Y contains m dye molecules, and subset Z contains o dye molecules, and wherein n, m and o are any integers (e.g., between 5 and 100). As described elsewhere herein, a nanostructure may contain 2, 3, 4, 5 or more photophysically-distinct subsets of dye molecules, each subset containing the same or different number of dye molecules.

Also provided herein are pluralities (e.g., at least two) of nucleic acid nanostructures, each nanostructure of the plurality comprising a unique set of dye molecules, which includes at least two photophysically-distinct subsets of dye molecules wherein the distance between dye molecules of a single photophysically-distinct subset is greater than the distance at which the dye molecules self-quench, and the distance between any pair of dye molecules, one dye molecule from one photophysically-distinct subset and the other dye molecule from another photophysically-distinct subset, is at least the Förster resonance energy transfer (FRET) radius of the pair of dye molecules. An example of a plurality of nucleic acid nanostructures of the present disclosure is shown in FIG. 3C. Each square represents a distinct nanostructure with a distinct set of dye molecules. That is, each nanostructure contains one or a unique combination of two or three photophysically-distinct subsets of dye molecules, resulting in a plurality of nanostructures having non-overlapping intensity distributions (see, e.g., FIG. 3A). An intensity distribution value for a single nanostructure is obtain by comparing that nanostructure to an established intensity distribution, recorded from multiple nanostructures having a known number of dye molecules. For example, if the intensity of 10 individual nanostructures is measured, each having 14 dye molecules, the result may be a distribution of intensity measurements having an upper limit of 100 units and a lower limit of 50 units. If the intensity of an additional nanostructure is measured (having an unknown number of dye molecules), and the intensity measurement is 75, then one can conclude that the additional nanostructure has 14 dye molecules. As another example, if the intensity of 10 individual nanostructures is measured, each having 27 dye molecules, the result may be a distribution of intensity measurements having an upper limit of 200 units and a lower limit of 120 units. If the intensity of an additional nanostructure is measured (having an unknown number of dye molecules), and the intensity measurement is 160, then one can conclude that the additional nanostructure has 27 dye molecules. Thus, in this example, a nanostructure containing 14 dye molecules and a different nanostructure containing 27 dye molecules have non-overlapping intensity distributions.

The nanostructure highlighted by a white dotted circle in FIG. 3C contains 27 red Alexa 647 dye molecules, 44 blue Atto 488 dye molecules and 27 green Cy3 dye molecules. The nanostructure directly below the white dotted circle contains 14 red Alexa 647 dye molecules, 44 blue Atto 488 dye molecules and 27 green Cy3 dye molecules. The nanostructure directly to the left of the white dotted circle contains 14 red Alexa 647 dye molecules, 44 blue Atto 488 dye molecules and 14 green Cy3 dye molecules. Thus, each nanostructure contains a unique “set” of dye molecules.

Within the plurality of nucleic acid nanostructures of FIG. 3C is a subset of nucleic acid nanostructures, wherein each nanostructure of the subset contains at least three photophysically-distinct subsets of dye molecules, each photophysically-distinct subset of dye molecules has a different number of dye molecules, and the intensity distributions of nucleic acid nanostructures of the subset are non-overlapping.

### Probes and Target Molecules

Metafluorophores of the present disclosure are typically used as detectable labels, or “tags.” For example, in some embodiments, metafluorophores are used to detect target molecules. Examples of probes and target molecules (e.g., binding partners) include, without limitation, proteins, saccharides (e.g., polysaccharides), lipids, nucleic acids (e.g., DNA, RNA, microRNAs, siRNAs), small molecules, organic and inorganic particles and/or surfaces. In some embodiments, target nucleic acids are antisense molecules, such as DNA antisense synthetic oligonucleotides (ASOs). Other probes and target molecules are contemplated.

Metafluorophores of the present disclosure, in some embodiments are attached to probes through a “handle” and “anti-handle” strand strategy, as described elsewhere herein. In some embodiments, metafluorophores are linked (e.g., covalently or non-covalently) to a probe through an intermediate linker molecule. In some embodiments, an intermediate linker includes an N-hydroxysuccinimide (NHS) linker. Other intermediate linkers may comprise biotin and/or streptavidin. For example, in some embodiments, a metafluorophore and a probe may each be biotinylated (i.e., linked to at least one biotin molecule) and linked to each other through biotin binding to an intermediate streptavidin molecule. Intermediate linkers provided herein may be used to link metafluorophores to probes, to link metafluorophores to dye molecules, or to link metafluorophores to substrates (e.g., glass).

### Triggered Assembly

Nucleic acid nanostructures of the present disclosure (metafluorophores) possess unique digitally programmable optical properties. Additionally, dynamical DNA nanotechnology makes it possible to program the formation of metafluorophores in an environmentally responsive fashion: metafluorophore can be programmed to form only upon detecting a user-specified trigger, for example. Triggered formation of metafluorophores are particularly useful for in situ imaging applications, for example: the fluorescent hairpin monomers, upon detecting a trigger attached to the target (e.g. an mRNA or a protein), form the metafluorophore attached to the trigger in situ. Compared with ex situ preformed metafluorophores, the in situ formed metafluorophores have at least two advantages. First, the monomer has a smaller size than the metafluorophore and thus can more easily penetrate into deep tissues with faster diffusion kinetics. Second, as the bright metafluorophore only forms at the target site, possible false positives caused by non-specific interactions of pre-assembled barcodes with cellular components can be avoided, and the signal amplification at the target site resulted from the triggered aggregation of fluorescent monomers will help to increase signal-to-background.

Thus, some aspects of the present disclosure provide systems (and kits) comprising a nucleic acid capture strand linked to a dye molecule, a nucleic acid trigger strand longer than the capture strand and comprising (a) a first domain that is complementary to the capture strand and (b) at least two concatenated domains, each of which comprises two subdomains, and a partially double-stranded nucleic acid comprising a single-stranded toehold domain having a nucleotide sequence complementary to one of the subdomains of the two subdomains of the concatenated domains, a double-stranded region linked to a dye molecule and having a nucleotide sequence complementary to the other of the two subdomains of the concatenated domains, and a single-stranded hairpin loop having a nucleotide sequence that is complementary to the single-stranded toehold domain.

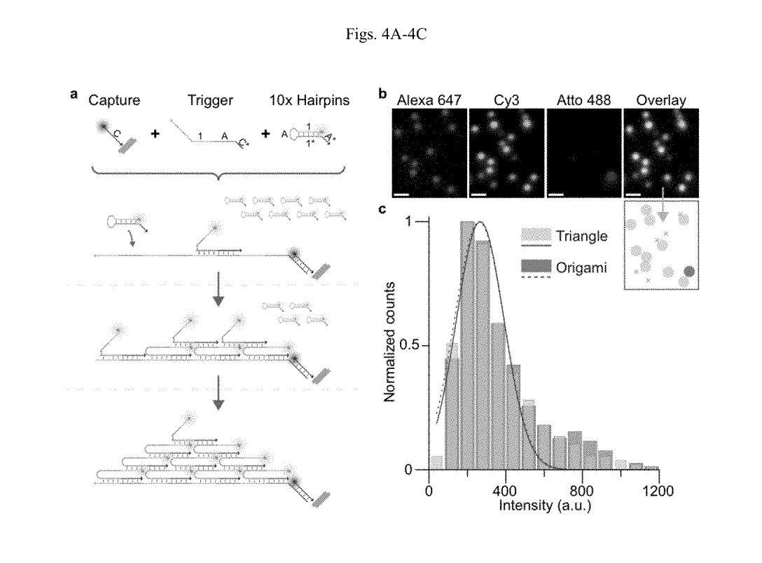

A “nucleic acid capture strand” refers to a single-stranded nucleic acid that is complementary to and binds to a “a nucleic acid trigger strand.” FIG. 4A depicts an example of a nucleic acid capture strand labeled with a dye molecule. A nucleic acid capture strand, in some embodiments, has a length of 5-100 nucleotides. For example, a nucleic acid capture strand may have a length of 5-90, 5-80, 5-70, 5-60, 5-50, 5-40, 5-30, 5-20, 10-100, 10-90, 10-80, 10-70, 10-60, 10-50, 10-40, 10-30, 10-20, 15-100, 15-90, 15-80, 15-70, 15-60, 15-50, 15-40, 15-30, 20-100, 20-90, 20-80, 20-70, 20-60, 20-50, 20-40, 20-30, 25-100, 25-90, 25-80, 25-70, 25-60, 25-50, 25-40, 25-30, 30-100, 30-90, 30-80, 30-70, 30-60, 30-50 or 30-40 nucleotides. In some embodiments, a nucleic acid capture strand has a length of 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 41, 42, 43, 44, 45, 46, 47, 48, 49 or 50 nucleotides. In some embodiments, a nucleic acid capture strand has a length of 15±5, 20±5, 25±5, 30±5, 35±5, 40±5, 45±5, or 15±5 nucleotides.

In some embodiments, a nucleic acid capture strand is attached to a surface (e.g., substrate or surface of a substrate. The substrate may be, for example, glass or other polymer. In some embodiments, a nucleic acid capture strand is attached to a surface via an linker. A linker may comprise, for example, biotin and/or streptavidin. Thus, in some embodiments, a (at least one) nucleic acid capture strand may be coupled to a surface, such as a glass surface.

In some embodiments, a nucleic acid capture strand is labeled with (comprises or is linked to) a dye molecule (a first dye molecule), as shown, for example, in FIG. 4A. Dye molecules (e.g., fluorescent molecules) are described elsewhere herein. Typically, the dye molecule of a capture strand is different from (not the same as) as dye molecule of the partially double-stranded nucleic acid described below.

A “nucleic acid trigger strand” refers to a single-stranded nucleic acid strand that comprises (a) a capture domain that is complementary to the capture strand (or complementary to a domain on the capture strand) and (b) at least two concatenated domains, each of which comprises two subdomains (see, e.g., FIG. 4A “Trigger,” where

“C*” denotes the capture domain, “1” denotes one of the subdomains (1 of 2) and “A” denotes the other of the subdomains (2 of 2). A nucleic acid trigger strand, in some embodiments, has a length of 100-5000 nucleotides. For example, a nucleic acid trigger strand may have a length of 100-4500, 100-4000, 100-3500, 100-3000, 100-2500, 100-2000, 100-1500, 100-1000, 100-500, 200-5000, 200-4500, 200-4000, 200-3500, 200-3000, 200-2500, 200-2000, 200-1500, 200-1000, or 200-500 nucleotides. In some embodiments, a nucleic acid trigger strand has a length of 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 nucleotides.

A “capture domain” of a nucleic acid trigger strand, in some embodiments, is complementary (fully (100%) complementary) to a capture strand, or a domain on the capture strand. In some embodiments, a capture domain is partially (less than 100%) complementary to a capture strand. In some embodiments, a capture domain has a length of 10-100 nucleotides. For example, a capture domain may have a length of 10-90, 10-80, 10-70, 10-60, 10-50, 10-40, 10-30, 10-20, 20-100, 20-90, 20-80, 20-70, 20-60, 20-50, 20-40, 20-30, 30-90, 30-80, 30-70, 30-60, 30-50, or 30-40 nucleotides. In some embodiments, a capture domain has a length of 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides.

A “concatenated domain” refers to a sequence of the nucleic acid that is repeated in a contiguous manner, as shown, for example, in FIG. 4A. A concatenated domain typically contains at least two subdomains, one of which is complementary to a toehold domain of a partially double-stranded nucleic acid (described below) and the other of which is complementary to a domain of the double-stranded region of a partially double-stranded nucleic acid (also described below). By way of example, FIG. 4A depicts a nucleic acid trigger strand containing four concatenated domains, each having a subdomain “1” and subdomain “A.” Subdomain “1” is complementary to domain “1*” of the partially double-stranded “Hairpin” nucleic acid, and subdomain “A” is complementary to toehold domain “A*” of the partially double-stranded nucleic acid. In some embodiments, a concatenated domain of a nucleic acid trigger strand has a length of 15-100 nucleotides. For example, a concatenated domain may have 15-90, 15-80, 15-70, 15-60, 15-50, 15-40, 15-30, 15-20, 20-100, 20-90, 20-80, 20-70, 20-60, 20-50, 20-40, 20-30, 30-100, 40-100, 40-90, 40-80, 40-70, 40-60, 40-50, 50-100, 50-90, 50-80, 50-70, or 50-60 nucleotides. In some embodiments, a concatenated domain has 10, 11, 12, 13, 14, 15, 16, 17, 18, 19, 20, 21, 22, 23, 24, 25, 26, 27, 28, 29 or 30 nucleotides.

In some embodiments, at least one of the two subdomains of a concatenated domain has a length of 5-50 nucleotides. For example, a subdomain may have a length of 5-40, 5-30, 5-20, 5-10, 10-50, 10-40, 10-30, 10-20, 15-50, 15-40, 15-30, or 15-10 nucleotides. In some embodiments, a subdomain has a length of 5, 6, 7, 8, 9,10, 11, 12, 13, 14, 15, 16, 17, 18, 19, 20, 21, 22, 23, 24, 25, 26, 27, 28, 29 or 30 nucleotides.

In some embodiments, one of the two subdomains of a concatenated domain is longer than the other of the two subdomains. For example, one subdomain (e.g., the 5′ subdomain) may be longer than the other subdomain (e.g., the 3′ subdomain) of a concatenated domain by 2-20 nucleotides. In some embodiments, one subdomain may be longer than another subdomain by 2, 3, 4, 5, 6, 7, 8, 9, 10, 11, 12, 13, 14, 15, 16, 17, 18, 19 or 20 nucleotides. In some embodiments, one subdomain may be at 10%-100% (e.g.,10%, 15%, 20%, 30%, 35%, 40%, 45%, 50%, 55%, 60%, 65%, 70%, 75%, 80%, 85%, 90%, 95% or 100%) longer than another subdomain.

A nucleic acid trigger strand, in some embodiments, comprises at least two concatenated domains. For example, a nucleic acid trigger strand may comprise at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 concatenated domains. In some embodiments, a nucleic acid trigger strand comprises 2-100 concatenated domains. For example, a nucleic acid trigger strand may comprise 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40 or 2-30 concatenated domains. In some embodiments, a nucleic acid trigger strand comprises 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95 or 100 concatenated domains.

A “partially double-stranded nucleic acid” refers to a nucleic acid strand that self-hybridizes to form a hairpin loop, as shown, for example, in FIG. 4A. A partially double-stranded nucleic acid comprises a single-stranded “toehold” domain having a nucleotide sequence complementary to one of the subdomains (e.g., a 3′ subdomain) of the two subdomains of the concatenated domains, a double-stranded region linked to a dye molecule and having a nucleotide sequence complementary to the other of the two subdomains (e.g., the 5′ subdomain) of the concatenated domains, and a single-stranded hairpin loop having a nucleotide sequence that is complementary to the single-stranded toehold domain and, thus, complementary to one of the subdomains (e.g., a 3′ subdomain) of the two subdomains of the concatenated domains of the trigger strand.

In some embodiments, a partially double-stranded nucleic acid has a length of 20-500 nucleotides. For example, a partially double-stranded nucleic acid may have a length of 20-400, 20-300, 20-200, 20-100, 20-90, 20-80, 20-70, 20-60, 20-50, 20-40, 30-500, 30-400, 30-300, 30-300, 30-100, 30-90, 30-80, 30-70, 30-60, 30-50, 30-40, 40-500, 40-400, 40-300, 40-400, 40-100, 40-90, 40-80, 40-70, 40-60, 40-50, 50-500, 50-400, 50-300, 50-500, 50-100, 50-90, 50-80, 50-70, or 50-60 nucleotides. In some embodiments, a partially double-stranded nucleic acid has a length of 20, 25, 30, 35, 40, 45, 50, 55, 60, 65, 70, 75, 80, 85, 90, 95 or 100 nucleotides.

In some embodiments, a single-stranded toehold domain has a length of 5-50 nucleotides. For example, a toehold domain may have a length of 5-40, 5-30, 5-20, 5-10, 10-50, 10-40, 10-30, 10-20, 15-50, 15-40, 15-30, or 15-10 nucleotides. In some embodiments, a toehold domain has a length of 5, 6, 7, 8, 9,10, 11, 12, 13, 14, 15, 16, 17, 18, 19, 20, 21, 22, 23, 24, 25, 26, 27, 28, 29 or 30 nucleotides.

In some embodiments, a double-stranded region has a length of 10-100 nucleotide base pairs. For example, a double-stranded region may have a length of 10-90, 10-80, 10-70, 10-60, 10-50, 10-40, 10-30, 10-20, 20-100, 20-90, 20-80, 20-70, 20-60, 20-50, 20-40, 20-30, 30-90, 30-80, 30-70, 30-60, 30-50, or 30-40 nucleotide base pairs. In some embodiments, a double-stranded region has a length of 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotide base pairs.

In some embodiments, a single-stranded hairpin loop has a length of 5-50 nucleotides. For example, a hairpin loop may have a length of 5-40, 5-30, 5-20, 5-10, 10-50, 10-40, 10-30, 10-20, 15-50, 15-40, 15-30, or 15-10 nucleotides. In some embodiments, a hairpin loop has a length of 5, 6, 7, 8, 9,10, 11, 12, 13, 14, 15, 16, 17, 18, 19, 20, 21, 22, 23, 24, 25, 26, 27, 28, 29 or 30 nucleotides.

A nucleic acid trigger strand is typically longer than a nucleic acid capture strand. For example, a trigger strand may be longer than a capture strand by 2-20 nucleotides. In some embodiments, a trigger strand is longer than a capture strand by 2, 3, 4, 5, 6, 7, 8, 9, 10, 11, 12, 13, 14, 15, 16, 17, 18, 19 or 20 nucleotides. In some embodiments, a trigger strand is longer than a capture strand by 10%-100% (e.g.,10%, 15%, 20%, 30%, 35%, 40%, 45%, 50%, 55%, 60%, 65%, 70%, 75%, 80%, 85%, 90%, 95% or 100%) longer than another subdomain.

In some embodiments, a (at least one) nucleic acid capture strand is attached to a surface (or a surface of a substrate). For example, 1-1000, 1-500, 1-100, 1-50, 1-25 or 1-10 nucleic acid capture strands may be attached to a surface. In some embodiments, 1, 2, 3, 4, 5, 6, 7, 8, 9, 10, 15, 20, 25, 30, 35, 40, 45, 50, 55, 60, 65, 70, 75, 80, 85, 90, 95, 100 or more nucleic acid capture strands are attached to a surface. In some embodiments, the surface is a glass surface.

In some embodiments, a system or kit of the present disclosure comprises at least two partially double-stranded hairpin nucleic acids. For example, a system or kit may comprise 2, 3, 4, 5, 6, 7, 8, 9, 10, 15, 20, 25, 30, 35, 40, 45, 50, 55, 60, 65, 70, 75, 80, 85, 90, 95, 100 or more partially double-stranded hairpin nucleic acids.

In some embodiments, at least one partially double-stranded nucleic acid is bound to the trigger nucleic acid, as shown, for example, in FIG. 4A.

In some embodiments, at least ten (e.g., 10-100) partially double-stranded nucleic acids are assembled on a single-stranded trigger nucleic acid bound to a single-stranded capture strand, thereby forming a nucleic acid nanostructure comprising at least 10 dye molecules.

FIG. 4A shows a schematic of an example of triggered assembly of triangular metafluorophores constructed from ten metastable Cy3-labeled DNA hairpin strands. A nucleic acid capture strand (labeled with Alexa 647) is attached to a glass surface through biotin-streptavidin coupling. A longer trigger strand hybridizes to the capture strand. The trigger strand in this example contains four concatenated domains ‘1-A,’ where the subdomain ‘1’ is 20 nucleotides in length, and subdomain ‘A’ is 12 nucleotides in length. Hairpin strands co-exist meta-stably in the absence of the trigger and only assemble into the desired structure upon exposure to the trigger. For example, the introduction of a repetitive single-stranded trigger initiates the assembly of kinetically trapped fluorescent hairpin monomers, which produce a second row of binding sites. These binding sites further enable the assembly of successive rows of monomers, with each row containing one fewer monomer than the previous. After assembly of 10 hairpins (labeled with Cy3) to a single trigger strand, no further trigger sequences are displayed and assembly is terminated, yielding a triangular-shaped metafluorophore of fixed dimensions. FIG. 4B shows fluorescence images of triangles assembled in situ on a glass surface. The capture strands are labeled with Alexa 647 and the hairpins with Cy3. DNA origami with 10 Cy3 and 44 Atto 488 dye molecules were added to the sample as intensity references. DNA origami structures can be identified at the positions where Atto 488 and Cy3 signals co-localize. In the schematic below the overlay fluorescence image, the one dark spot represents the Atto 488-labeled origami marker, and the lighter gray spots represent the expected overlay of Alexa 647-labeled capture strand and the triangle composed of Cy3-labeled hairpin monomers. The gray “x” symbols represent non-specific binding of hairpins to the surface. FIG. 4C shows that triangular metafluorophores (light gray) and reference DNA origami (dark gray) intensity distributions are overlapping, indicating the formation of the triangles.

Also provided herein are methods of assembling a metafluorophore, comprising contacting a surface containing a plurality of capture strands with a trigger strand and a plurality of partially double-stranded hairpins under conditions that result in self-assembly (hybridization) of the partially double-stranded hairpins into a metafluorophore.

### Additional Embodiments

I: Dualcolor FRET, Geometrical Encoding, Dyes are Only Distinct by their Spectrum

Provided herein are nucleic acid nanostructures that comprise at least two spectrally-distinct subsets of dye molecules, wherein the distance between any pair of dye molecules, one dye molecule from one spectrally-distinct subset and the other dye molecule from another spectrally-distinct subset, is within the distance where Förster resonance energy transfer (FRET) does take place.

In some embodiments the donor dye of such a FRET pair has one acceptor dye in it's immediate vicinity. (R1-G1)

In some embodiments the donor dye of such a FRET pair has several acceptor dyes in its immediate vicinity. (R1-G1-R1)

II: Multicolor FRET, Geometrical Encoding, Dyes are Only Distinct by their Spectrum

Also provided herein are nucleic acid nanostructures that comprise at least three spectrally-distinct subsets of dye molecules, wherein the distance between any pair of dye molecules, one dye molecule from one spectrally-distinct subset and the other dye molecule from another spectrally-distinct subset, is within the distance where Förster resonance energy transfer (FRET) does take place.

In some embodiments the donor dye of such a FRET pair has one acceptor dye of one of the subset of spectrally distinct dye molecules in its immediate vicinity. (R1-G1 ̂ R1-B1)

In some embodiments the donor dye of such a FRET pair has several acceptor dyes of one of the subset of spectrally distinct dye molecules in its immediate vicinity. (R1-G1-R1 ̂ R1-B1-R1)

In some embodiments the donor dye of such a FRET pair has several acceptor dyes of several subsets of spectrally distinct dye molecules in its immediate vicinity. (e.g. R1-G1-B1)

### III: Dualcolor FRET, Geometrical Encoding, Dyes are Distinct by Photokinetics

Provided herein are nucleic acid nanostructures that comprise at least three photophysically-distinct subsets of dye molecules, wherein at least two of the photophysically distinct subset of dye molecules are spectrally overlapping and wherein the distance between any pair of dye molecules, one dye molecule from one spectrally-distinct subset and the other dye molecule from another spectrally-distinct subset, is within the distance where Förster resonance energy transfer (FRET) does take place.

In some embodiments the donor dye of such a FRET pair has one acceptor dye of a spectrally distinct subset in its immediate vicinity. (R1-G1 ̂ R2-G1)

In some embodiments the donor dye of such a FRET pair has several acceptor dyes of one of the spectrally distinct subsets in its immediate vicinity. (R1-G1-R1 ̂ R2-G1-R1)

In some embodiments the donor dye of such a FRET pair has several acceptor dyes of any of the spectrally distinct subsets in its immediate vicinity. (R1-G1-R2)

### IV: Multicolor FRET, Geometrical Encoding, Dyes are Distinct by Spectrum and Photokinetics

Also provided herein are nucleic acid nanostructures that comprise at least three photophysically-distinct subsets of dye molecules, wherein the distance between any pair of dye molecules, one dye molecule from one spectrally-distinct subset and the other dye molecule from another spectrally-distinct subset, is within the distance where Förster resonance energy transfer (FRET) does take place.

In some embodiments the donor dye of such a FRET pair has one acceptor dye of a photophysically distinct subset in its immediate vicinity. (R1-G1 ̂ R2-G1 ̂ R1-B1 ̂ R2-B1)

In some embodiments the donor dye of such a FRET pair has several acceptor dyes of one of the photophysically distinct subsets in its immediate vicinity. (R1-G1-R1 ̂ R2-G1-R2 ̂ R1-B1-R1 ̂ R2-B1R2)

In some embodiments the donor dye of such a FRET pair has several acceptor dyes of any of the photophysically distinct subsets in its immediate vicinity. (R1-G1-R2 ̂ R1-B1-R2)

Also provided herein are pluralities (e.g., at least two) of nucleic acid nanostructures, each nanostructure of the plurality comprising a unique set of dye molecules.

### Applications and Kits

Metafluorophores of the present disclosure may be used as labels for probes, for example, for multiplexed target detection, fluorescence correlation spectroscopy (FCS), flow cytometry, and signal amplification with microscopy though high-density labeling.

In some embodiments, methods include capturing a target molecule, such as DNA or RNA, on a surface of a substrate (e.g., a glass substrate), contacting the captured targets with barcoded metafluorophores as provided herein, and identifying the targets via fluorescence microscopy. Other applications are contemplated herein.

Aspects of the present disclosure also provide kits comprising any two or more components or reagents, as provided herein.

Additional aspects of the present disclosure are encompassed by the following numbered paragraphs:

1. A nucleic acid nanostructure, comprising at least two photophysically-distinct subsets of dye molecules, wherein the distance between dye molecules of a single photophysically-distinct subset is greater than the distance at which the dye molecules self-quench, and the distance between any pair of dye molecules, one dye molecule from one photophysically-distinct subset and the other dye molecule from another photophysically-distinct subset, is at least the Förster resonance energy transfer (FRET) radius of the pair of dye molecules.

2. The nucleic acid nanostructure of paragraph 1, wherein the distance between any pair of dye molecules of a single photophysically-distinct subset is at least 5 nm.

3. The nucleic acid nanostructure of paragraph 2, wherein the distance between any pair of dye molecules of a single photophysically-distinct subset is 5 nm to 100 nm.

4. The nucleic acid nanostructure of any one of paragraphs 1-3, wherein the distance between any pair of dye molecules, one dye molecule from one photophysically-distinct subset and the other dye molecule from another photophysically-distinct subset, is at least 10 nm.

5. The nucleic acid nanostructure of paragraph 4, wherein the distance between any pair of dye molecules, one dye molecule from one photophysically-distinct subset and the other dye molecule from another photophysically-distinct subset, is 10 nm to 100 nm.

6. The nucleic acid nanostructure of any one of paragraphs 1-5, wherein the nucleic acid nanostructure has a size of 5 nm to 200 nm.

7. The nucleic acid nanostructure of any one of paragraphs 1-6, wherein dye molecules of each photophysically-distinct subset are attached indirectly to a nucleic acid of the nanostructure.

8. The nucleic acid nanostructure of paragraph 7, wherein dye molecules of each photophysically-distinct subset are attached indirectly to a nucleic acid of the nanostructure through at least one single-stranded nucleic acid.

9. The nucleic acid nanostructure of paragraph 8, wherein the at least one single-stranded nucleic is 15 to 100 nucleotides in length.

10. The nucleic acid nanostructure of any one of paragraphs 1-9, wherein dye molecules of a single photophysically-distinct subset are grouped together within a defined region on the nanostructure.

11. The nucleic acid nanostructure of any one of paragraphs 1-10, comprising at least three photophysically-distinct subsets of dye molecules.

12. The nucleic acid nanostructure of paragraph 11, comprising three to ten photophysically-distinct subsets of dye molecules.

13. The nucleic acid nanostructure of any one of paragraphs 1-12, wherein the photophysically-distinct subsets of dye molecules are spectrally-distinct subsets of dye molecules.

14. The nucleic acid nanostructure of any one of paragraphs 1-12, wherein the photophysically-distinct subsets of dye molecules have different bleaching kinetics relative to each other.

15. The nucleic acid nanostructure of any one of paragraphs 1-12, wherein the photophysically-distinct subsets of dye molecules have different photoswitchable properties relative to each other.

16. A plurality of nucleic acid nanostructures, each nanostructure comprising a unique set of dye molecules, wherein each set of dye molecules includes at least two photophysically-distinct subsets of dye molecules, wherein the distance between dye molecules of a single photophysically-distinct subset is greater than the distance at which the dye molecules self-quench, and the distance between any pair of dye molecules, one dye molecule from one photophysically-distinct subset and the other dye molecule from another photophysically-distinct subset, is at least the Förster resonance energy transfer (FRET) radius of the pair of dye molecules.

17. The plurality of nucleic acid nanostructures of paragraph 16, wherein the nucleic acid nanostructures have non-overlapping intensity distributions.

18. The plurality of nucleic acid nanostructures of paragraph 16 or 17, wherein the distance between any pair of dye molecules of a single photophysically-distinct subset is at least 5 nm.

19. The plurality of nucleic acid nanostructures of paragraph 18, wherein the distance between any pair of dye molecules of a single photophysically-distinct subset is 5 nm to 50 nm.

20. The plurality of nucleic acid nanostructures of any one of paragraphs 16-19, wherein on a single nanostructure the distance between any pair of dye molecules, one dye molecule from one photophysically-distinct subset and the other dye molecule from another photophysically-distinct subset, is at least 10 nm.

21. The plurality of nucleic acid nanostructures of paragraph 20, wherein on a single nanostructure the distance between any pair of dye molecules, one dye molecule from one photophysically-distinct subset and the other dye molecule from another photophysically-distinct subset, is 10 nm to 100 nm.

22. The plurality of nucleic acid nanostructures of any one of paragraphs 16-21, wherein the nucleic acid nanostructures have a size of less than 200 nm.

23. The plurality of nucleic acid nanostructures of any one of paragraphs 16-22, wherein dye molecules of each photophysically-distinct subset are attached indirectly to a nucleic acid of the nanostructure.

24. The plurality of nucleic acid nanostructures of any one of paragraphs 16-23, wherein dye molecules of each photophysically-distinct subset are attached indirectly to a nucleic acid of a nanostructure of the plurality via at least one single-stranded nucleic acid.

25. The plurality of nucleic acid nanostructures of paragraph 24, wherein the at least one single-stranded nucleic is 15 to 100 nucleotides in length.

26. The plurality of nucleic acid nanostructures of any one of paragraphs 16-25, wherein dye molecules of a single photophysically-distinct subset are grouped together within a defined region on a nanostructure of the plurality.

27. The plurality of nucleic acid nanostructures of any one of paragraphs 16-26, wherein each set of dye molecules on a nanostructure comprises at least three photophysically-distinct subsets of dye molecules.

28. The plurality of nucleic acid nanostructures of paragraph 27, wherein each set of dye molecules on a nanostructure comprises three to ten photophysically-distinct subsets of dye molecules.

29. The plurality of nucleic acid nanostructures of any one of paragraphs 16-28, wherein the photophysically-distinct subsets of dye molecules are spectrally-distinct subsets of dye molecules.

30. The plurality of nucleic acid nanostructures of any one of paragraphs 16-28, wherein the photophysically-distinct subsets of dye molecules have different bleaching kinetics relative to each other.

31. The plurality of nucleic acid nanostructures of any one of paragraphs 16-28, wherein the photophysically-distinct subsets of dye molecules have different photoswitchable properties relative to each other.

32. A subset of nucleic acid nanostructures of the plurality of any one of paragraphs 16-31, wherein each nanostructure of the subset contains at least three photophysically-distinct subsets of dye molecules, each photophysically-distinct subset of dye molecules has a different number of dye molecules, and the intensity distributions of nucleic acid nanostructures of the subset are non-overlapping.

33. The nucleic acid nanostructure of any one of paragraphs 1-15 linked to a first single-stranded oligonucleotide that is complementary to a first region of a nucleic acid target.

34. The nucleic acid nanostructure of paragraph 33, wherein the first single-stranded oligonucleotide is bound to the first region of a nucleic acid target.

35. The nucleic acid nanostructure of paragraph 34, wherein the nucleic acid target comprises a second region complementary to and bound to a second single-stranded oligonucleotide, wherein the second single-stranded oligonucleotide is attached to a substrate.

36. The nucleic acid nanostructure of paragraph 35, wherein the second single-stranded oligonucleotide is biotinylated.

37. The nucleic acid nanostructure of paragraph 36, wherein the surface is coated in streptavidin and the second biotinylated single-stranded oligonucleotide is attached to the substrate via a biotin-streptavidin binding interaction.

38. The nucleic acid nanostructure of any one of paragraphs 35-37, wherein the substrate is a glass or plastic substrate.

39. A substrate comprising on a surface of the substrate a plurality of biotinylated single-stranded oligonucleotides, wherein at least some of the biotinylated single-stranded oligonucleotides are complementary to and bound to a region of a target nucleic acid, and wherein the first single-stranded oligonucleotide of the nucleic acid nanostructure of paragraph 33 is complementary to and bound to another region of the target nucleic acid.

40. A method of quantifying nucleic acid targets, comprising

(a) applying target nucleic acids to a substrate comprising on a surface of the substrate a plurality of biotinylated single-stranded oligonucleotides, wherein the target nucleic acids comprise a first and second region, and wherein the biotinylated single-stranded oligonucleotides are complementary to the second region of the target nucleic acids; (b) applying to the substrate of (a) a plurality of the nucleic acid nanostructures of paragraph 33 under conditions that result in binding of the nucleic acid nanostructures to nucleic acid targets; and (c) quantifying nucleic acid nanostructures bound to nucleic acid targets.

41. A system (or kit) comprising a nucleic acid capture strand linked to a first dye molecule; a nucleic acid trigger strand longer than the capture strand and comprising (a) a capture domain that is complementary to the capture strand and (b) at least two concatenated domains, each of which comprises two subdomains; and a partially double-stranded nucleic acid comprising a single-stranded toehold domain having a nucleotide sequence complementary to one of the subdomains of the two subdomains of the concatenated domains, a double-stranded region linked to a second dye molecule and having a nucleotide sequence complementary to the other of the two subdomains of the concatenated domains, and a single-stranded hairpin loop having a nucleotide sequence that is complementary to the single-stranded toehold domain.

42. The system (or kit) of paragraph 41, wherein the nucleic acid capture strand has a length of 10-100 nucleotides.

43. The system (or kit) of paragraph 41 or 42, wherein the dye molecule is a fluorescent dye molecule.

44. The system (or kit) of any one of paragraphs 41-43, wherein the nucleic acid trigger strand has a length of 100-5000 nucleotides.

45. The system (or kit) of paragraph 44, wherein the nucleic acid trigger strand has a length of 100-1000 nucleotides.

46. The system (or kit) of any one of paragraphs 41-45, wherein the capture domain has a length of 10-100 nucleotides.

47. The system (or kit) of any one of paragraphs 41-46, wherein a concatenated domain of a nucleic acid trigger strand has a length of 15-100 nucleotides.

48. The system (or kit) of any one of paragraphs 41-47, wherein at least one of the two subdomains of a concatenated domain has a length of 5-50 nucleotides.

49. The system (or kit) of any one of paragraphs 41-48, wherein one of the two subdomains of a concatenated domain is longer than the other of the two subdomains.

50. The system (or kit) of any one of paragraphs 41-49, wherein the partially double-stranded nucleic acid has a length of 20-500 nucleotides.

51. The system (or kit) of any one of paragraphs 41-50, wherein the single-stranded toehold domain has a length of 5-50 nucleotides.

52. The system (or kit) of any one of paragraphs 41-51, wherein the double-stranded region has a length of 10-100 nucleotides.

53. The system (or kit) of any one of paragraphs 41-52, wherein the single-stranded hairpin loop has a length of 5-50 nucleotides.

54. The system (or kit) of any one of paragraphs 41-53, wherein the nucleic acid capture strand is attached to a substrate.

55. The system (or kit) of any one of paragraphs 41-54 further comprising at least two partially double-stranded nucleic acids.

56. The system (or kit) of paragraph 55 further comprising at least ten partially double-stranded nucleic acids.

57. The system (or kit) of any one of paragraphs 41-56, wherein at least one partially double-stranded nucleic acid is bound to the trigger nucleic acid.

58. The system (or kit) of paragraph 56, wherein at least ten partially double-stranded nucleic acids are assembled on a single-stranded trigger nucleic acid bound to a single-stranded capture strand, thereby forming a nucleic acid nanostructure comprising at least 10 dye molecules.

### EXAMPLES

### Example 1

### Designing a Metafluorophore Using DNA Nanostructures

The instant Example provides a nucleic acid-based platform for assembling nanoscale metafluorophores with programmable properties. DNA origami, for example, utilizes a long single-stranded DNA molecule (referred to as the “scaffold”), folded into programmable shapes by ˜200 short, single-stranded DNA strands (referred to as “staples”). Every staple has a defined sequence and specifically binds certain parts of the scaffold together. Nanostructures are usually assembled in a one-pot reaction using thermal annealing. After the self-assembly is completed, the scaffold is “folded” into the desired shape with the staple strands at prescribed positions in the final origami.

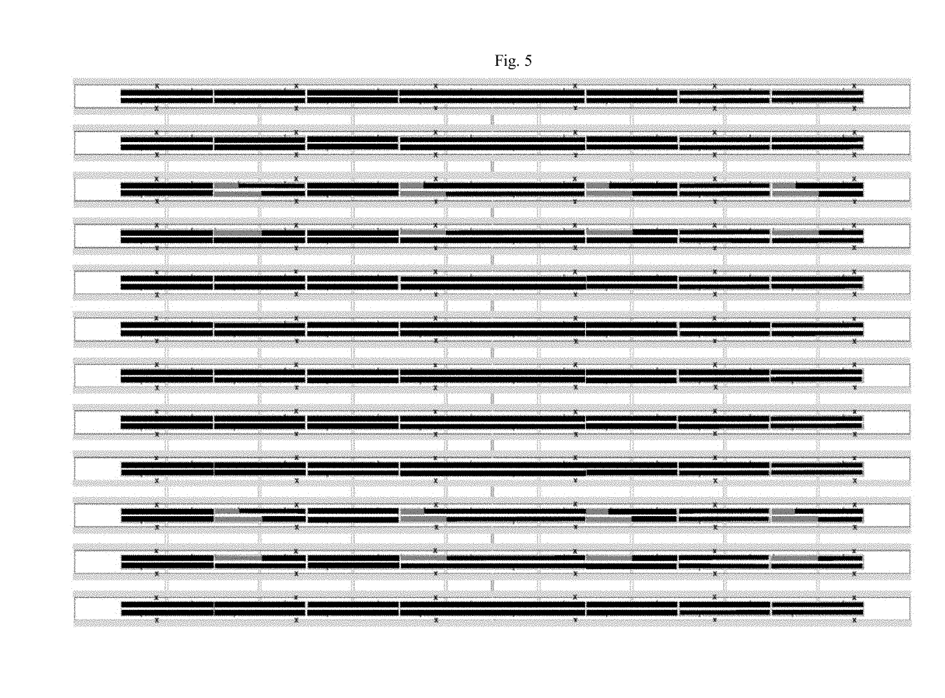

A two-dimensional, rectangular DNA nanostructure, containing of 24 parallel DNA double helices with dimensions of 90×60 nm², was used (FIG. 1A, FIG. 5, Table 1 and the M13mp18 scaffold sequence (SEQ ID NO: 185)). This nanostructure contained 184 uniquely addressable staple strands (sequences shown in Table 1). The designated colors match those depicted in the caDNAno layout shown in FIG. 5. The first column denotes the position of the staple strand according to the caDNAno layout. The first digit indicates the helix on which the 5′-end is located (y-coordinate), the succeeding number in brackets marks the number of base pairs between the boundary and the 5′-end (x-coordinate). The second pair of numbers corresponds to the 3′-end in similar fashion.

TABLE 1
DNA origami staple sequences.

| | | | | SEQ |
|---|---|---|---|---|
| Position | Sequence | Color | Description | ID NO: |
| 0[111]1[95] | TAAATGAATTTTCTGTATGGGA | gray | Structure strand | 1 |
| | TTAATTTCTT | | | |
| 0[143]1[127] | TCTAAAGTTTTGTCGTCTTTCCA | gray | Structure strand | 2 |
| | GCCGACAA | | | |
| 0[175]0[144] | TCCACAGACAGCCCTCATAGTT | gray | Structure strand | 3 |
| | AGCGTAACGA | | | |
| 0[207]1[191] | TCACCAGTACAAACTACAACGC | gray | Structure strand | 4 |
| | CTAGTACCAG | | | |
| 0[239]1[223] | AGGAACCCATGTACCGTAACA | gray | Structure strand | 5 |
| | CTTGATATAA | | | |
| 0[271]1[255] | CCACCCTCATTTTCAGGGATAG | gray | Structure strand | 6 |
| | CAACCGTACT | | | |
| 0[47]1[31] | AGAAAGGAACAACTAAAGGAA | gray | Structure strand | 7 |
| | TTCAAAAAAA | | | |
| 0[79]1[63] | ACAACTTTCAACAGTTTCAGCG | gray | Structure strand | 8 |
| | GATGTATCGG | | | |
| 1[128]4[128] | TGACAACTCGCTGAGGCTTGCA | gray | Structure strand | 9 |
| | TTATACCAAGCGCGATGATAAA | | | |
| 1[160]2[144] | TTAGGATTGGCTGAGACTCCTC | gray | Structure strand | 10 |
| | AATAACCGAT | | | |
| 1[192]4[192] | GCGGATAACCTATTATTCTGAA | gray | Structure strand | 11 |
| | ACAGACGATTGGCCTTGAAGA | | | |
| | GCCAC | | | |
| 1[192]4[192] | GTATAGCAAACAGTTAATGCCC | gray | Structure strand | 12 |
| | AATCCTCA | | | |
| 1[256]4[256] | CAGGAGGTGGGGTCAGTGCCTT | gray | Structure strand | 13 |
| | GAGTCTCTGAATTTACCGGGAA | | | |
| | CCAG | | | |
| 1[32]3[31] | AGGCTCCAGAGGCTTTGAGGA | gray | Structure strand | 14 |
| | CACGGGTAA | | | |
| 1[64]4[64] | TTTATCAGGACAGCATCGGAAC | gray | Structure strand | 15 |
| | GACACCAACCTAAAACGAGGT | | | |
| | CAATC | | | |
| 1[96]3[95] | AAACAGCTTTTTGCGGGATCGT | gray | Structure strand | 16 |
| | CAACACTAAA | | | |
| 10[111]8[112] | TTGCTCCTTTCAAATATCGCGT | gray | Structure strand | 17 |
| | TTGAGGGGGT | | | |
| 10[143]9[159] | CCAACAGGAGCGAACCAGACC | gray | Structure strand | 18 |
| | GGAGCCTTTAC | | | |
| 10[175]8[176] | TTAACGTCTAACATAAAAACAG | gray | Structure strand | 19 |
| | GTAACGGA | | | |
| 10[207]8[208] | ATCCCAATGAGAATTAACTGAA | gray | Structure strand | 20 |
| | CAGTTACCAG | | | |
| 10[239]8[240] | GCCAGTTAGAGGGTAATTGAG | gray | Structure strand | 21 |
| | CGCTTTAAGAA | | | |
| 10[271]8[272] | ACGCTAACACCCACAAGAATT | gray | Structure strand | 22 |
| | GAAAATAGC | | | |
| 10[47]8[48] | CTGTAGCTTGACTATTATAGTC | gray | Structure strand | 23 |
| | AGTTCATTGA | | | |
| 10[79]8[80] | GATGGCTTATCAAAAAGATTAA | gray | Structure strand | 24 |
| | GAGCGTCC | | | |
| 11[128]13[127] | TTTGGGGATAGTAGTAGCATTA | gray | Structure strand | 25 |
| | AAAGGCCG | | | |
| 11[160]12[144] | CCAATAGCTCATCGTAGGAATC | gray | Structure strand | 26 |
| | ATGGCATCAA | | | |
| 11[192]13[191] | TATCCGGTCTCATCGAGAACAA | gray | Structure strand | 27 |
| | GCGACAAAAG | | | |
| 11[224]13[223] | GCGAACCTCCAAGAACGGGTA | gray | Structure strand | 28 |
| | TGACAATAA | | | |
| 11[256]13[255] | GCCTTAAACCAATCAATAATCG | gray | Structure strand | 29 |
| | GCACGCGCCT | | | |
| 11[32]13[31] | AACAGTTTTGTACCAAAAACAT | gray | Structure strand | 30 |
| | TTTATTTC | | | |
| 11[64]13[63] | GATTTAGTCAATAAAGCCTCAG | gray | Structure strand | 31 |
| | AGAACCCTCA | | | |
| 11[96]13[95] | AATGGTCAACAGGCAAGGCAA | gray | Structure strand | 32 |
| | AGAGTAATGTG | | | |
| 12[111]10[112] | TAAATCATATAACCTGTTTAGC | gray | Structure strand | 33 |
| | TAACCTTTAA | | | |
| 12[143]11[159] | TTCTACTACGCGAGCTGAAAAG | gray | Structure strand | 34 |
| | GTTACCGCGC | | | |
| 12[175]10[176] | TTTTATTTAAGCAAATCAGATA | gray | Structure strand | 35 |
| | TTTTTTGT | | | |
| 12[207]10[208] | GTACCGCAATTCTAAGAACGCG | gray | Structure strand | 36 |
| | AGTATTATTT | | | |
| 12[239]10[240] | CTTATCATTCCCGACTTGCGGG | gray | Structure strand | 37 |
| | AGCCTAATTT | | | |
| 12[271]10[272] | TGTAGAAATCAAGATTAGTTGC | gray | Structure strand | 38 |
| | TCTTACCA | | | |
| 12[47]10[48] | TAAATCGGGATTCCCAATTCTG | gray | Structure strand | 39 |
| | CGATATAATG | | | |
| 12[79]10[80] | AAATTAAGTTGACCATTAGATA | gray | Structure strand | 40 |
| | CTTTTGCG | | | |
| 13[128]15[127] | GAGACAGCTAGCTGATAAATT | gray | Structure strand | 41 |
| | AATTTTTGT | | | |
| 13[160]14[144] | GTAATAAGTTAGGCAGAGGCA | gray | Structure strand | 42 |
| | TTTATGATATT | | | |
| 13[192]15[191] | GTAAAGTAATCGCCATATTTAA | gray | Structure strand | 43 |
| | CAAAACTTTT | | | |
| 13[224]15[223] | ACAACATGCCAACGCTCAACA | gray | Structure strand | 44 |
| | GTCTTCTGA | | | |
| 13[256]15[255] | GTTTATCAATATGCGTTATACA | gray | Structure strand | 45 |
| | AACCGACCGT | | | |
| 13[32]15[31] | AACGCAAAATCGATGAACGGT | gray | Structure strand | 46 |
| | ACCGGTTGA | | | |
| 13[64]15[63] | TATATTTTGTCATTGCCTGAGA | gray | Structure strand | 47 |
| | GTGGAAGATT | | | |
| 13[96]15[95] | TAGGTAAACTATTTTTGAGAGA | gray | Structure strand | 48 |
| | TCAAACGTTA | | | |
| 14[111]12[112] | GAGGGTAGGATTCAAAAGGGT | gray | Structure strand | 49 |
| | GAGACATCCAA | | | |
| 14[143]13[159] | CAACCGTTTCAAATCACCATCA | gray | Structure strand | 50 |
| | ATTCGAGCCA | | | |
| 14[175]12[176] | CATGTAATAGAATATAAAGTAC | gray | Structure strand | 51 |
| | CAAGCCGT | | | |
| 14[207]12[208] | AATTGAGAATTCTGTCCAGACG | gray | Structure strand | 52 |
| | ACTAAACCAA | | | |
| 14[239]12[240] | AGTATAAAGTTCAGCTAATGCA | gray | Structure strand | 53 |
| | GATGTCTTTC | | | |
| 14[271]12[272] | TTAGTATCACAATAGATAAGTC | gray | Structure strand | 54 |
| | CACGAGCA | | | |
| 14[47]12[48] | AACAAGAGGGATAAAAATTTT | gray | Structure strand | 55 |
| | TAGCATAAAGC | | | |
| 14[79]12[80] | GCTATCAGAAATGCAATGCCTG | gray | Structure strand | 56 |
| | AATTAGCA | | | |
| 15[128]18[128] | TAAATCAAAATAATTCGCGTCT | gray | Structure strand | 57 |
| | CGGAAACCAGGCAAAGGGAAG | | | |
| | G | | | |
| 15[160]16[144] | ATCGCAAGTATGTAAATGCTGA | gray | Structure strand | 58 |
| | TGATAGGAAC | | | |
| 15[192]18[192] | TCAAATATAACCTCCGGCTTAG | gray | Structure strand | 59 |
| | GTAACAATTTCATTTGAAGGCG | | | |
| | AATT | | | |
| 15[224]17[223] | CCTAAATCAAAATCATAGGTCT | gray | Structure strand | 60 |
| | AAACAGTA | | | |
| 15[256]18[256] | GTGATAAAAAGACGCTGAGAA | gray | Structure strand | 61 |
| | GAGATAACCTTGCTTCTGTTCG | | | |
| | GGAGA | | | |
| 15[32]17[31] | TAATCAGCGGATTGACCGTAAT | gray | Structure strand | 62 |
| | CGTAACCG | | | |
| 15[64]18[64] | GTATAAGCCAACCCGTCGGATT | gray | Structure strand | 63 |
| | CTGACGACAGTATCGGCCGCA | | | |
| | AGGCG | | | |
| 15[96]17[95] | ATATTTTGGCTTTCATCAACAT | gray | Structure strand | 64 |
| | TATCCAGCCA | | | |
| 16[111]14[112] | TGTAGCCATTAAAATTCGCATT | gray | Structure strand | 65 |
| | AAATGCCGGA | | | |
| 16[143]15[159] | GCCATCAAGCTCATTTTTTAAC | gray | Structure strand | 66 |
| | CACAAATCCA | | | |
| 16[175]14[176] | TATAACTAACAAAGAACGCGA | gray | Structure strand | 67 |
| | GAACGCCAA | | | |
| 16[207]14[208] | ACCTTTTTATTTTAGTTAATTTC | gray | Structure strand | 68 |
| | ATAGGGCTT | | | |
| 16[239]14[240] | GAATTTATTTAATGGTTTGAAA | gray | Structure strand | 69 |
| | TATTCTTACC | | | |
| 16[271]14[272] | CTTAGATTTAAGGCGTTAAATA | gray | Structure strand | 70 |
| | AAGCCTGT | | | |
| 16[47]14[48] | ACAAACGGAAAAGCCCCAAAA | gray | Structure strand | 71 |
| | ACACTGGAGCA | | | |
| 16[79]14[80] | GCGAGTAAAAATATTTAAATTG | gray | Structure strand | 72 |
| | TTACAAAG | | | |
| 17[160]18[144] | AGAAAACAAAGAAGATGATGA | gray | Structure strand | 73 |
| | AACAGGCTGCG | | | |
| 17[224]19[223] | CATAAATCTTTGAATACCAAGT | gray | Structure strand | 74 |
| | GTTAGAAC | | | |
| 17[32]19[31] | TGCATCTTTCCCAGTCACGACG | gray | Structure strand | 75 |
| | GCCTGCAG | | | |
| 17[96]19[95] | GCTTTCCGATTACGCCAGCTGG | gray | Structure strand | 76 |
| | CGGCTGTTTC | | | |
| 18[111]16[112] | TCTTCGCTGCACCGCTTCTGGT | gray | Structure strand | 77 |
| | GCGGCCTTCC | | | |
| 18[143]14[159] | CAACTGTTGCGCCATTCGCCAT | gray | Structure strand | 78 |
| | TCAAACATCA | | | |
| 18[175]16[176] | CTGAGCAAAAATTAATTACATT | gray | Structure strand | 79 |
| | TTGGGTTA | | | |
| 18[207]16[208] | CGCGCAGATTACCTTTTTTAAT | gray | Structure strand | 80 |
| | GGGAGAGACT | | | |
| 18[239]16[240] | CCTGATTGCAATATATGTGAGT | gray | Structure strand | 81 |
| | GATCAATAGT | | | |
| 18[271]16[272] | CTTTTACAAAATCGTCGCTATT | gray | Structure strand | 82 |
| | AGCGATAG | | | |
| 18[47]16[48] | CCAGGGTTGCCAGTTTGAGGGG | gray | Structure strand | 83 |
| | ACCCGTGGGA | | | |
| 18[79]16[80] | GATGTGCTTCAGGAAGATCGCA | gray | Structure strand | 84 |
| | CAATGTGA | | | |
| 19[160]20[144] | GCAATTCACATATTCCTGATTA | gray | Structure strand | 85 |
| | TCAAAGTGTA | | | |
| 19[224]21[223] | CTACCATAGTTTGAGTAACATT | gray | Structure strand | 86 |
| | TAAAATAT | | | |
| 19[32]21[31] | GTCGACTTCGGCCAACGCGCGG | gray | Structure strand | 87 |
| | GGTTTTTC | | | |
| 19[96]21[95] | CTGTGTGATTGCGTTGCGCTCA | gray | Structure strand | 88 |
| | CTAGAGTTGC | | | |
| 2[111]0[112] | AAGGCCGCTGATACCGATAGTT | gray | Structure strand | 89 |
| | GCGACGTTAG | | | |
| 2[143]1[159] | ATATTCGGAACCATCGCCCACG | gray | Structure strand | 90 |
| | CAGAGAAGGA | | | |
| 2[175]0[176] | TATTAAGAAGCGGGGTTTTGCT | gray | Structure strand | 91 |
| | CGTAGCAT | | | |
| 2[207]0[208] | TTTCGGAAGTGCCGTCGAGAGG | gray | Structure strand | 92 |
| | GTGAGTTTCG | | | |
| 2[239]0[240] | GCCCGTATCCGGAATAGGTGTA | gray | Structure strand | 93 |
| | TCAGCCCAAT | | | |
| 2[271]0[272] | GTTTTAACTTAGTACCGCCACC | gray | Structure strand | 94 |
| | CAGAGCCA | | | |
| 2[47]0[48] | ACGGCTACAAAAGGAGCCTTT | gray | Structure strand | 95 |
| | AATGTGAGAAT | | | |
| 2[79]0[80] | CAGCGAAACTTGCTTTCGAGGT | gray | Structure strand | 96 |
| | GTTGCTAA | | | |
| 20+111+18+112+ | CACATTAAAATTGTTATCCGCT | gray | Structure strand | 97 |
| | CATGCGGGCC | | | |
| 20[143]19[159] | AAGCCTGGTACGAGCCGGAAG | gray | Structure strand | 98 |
| | CATAGATGATG | | | |
| 20[175]18[176] | ATTATCATTCAATATAATCCTG | gray | Structure strand | 99 |
| | ACAATTAC | | | |
| 20[207]18[208] | GCGGAACATCTGAATAATGGA | gray | Structure strand | 100 |
| | AGGTACAAAAT | | | |
| 20[239]18[240] | ATTTTAAAATCAAAATTATTTG | gray | Structure strand | 101 |
| | CACGGATTCG | | | |
| 20[271]18[272] | CTCGTATTAGAAATTGCGTAGA | gray | Structure strand | 102 |
| | TACAGTAC | | | |
| 20[47]18[48] | TTAATGAACTAGAGGATCCCCG | gray | Structure strand | 103 |
| | GGGGGTAACG | | | |
| 20[79]18[80] | TTCCAGTCGTAATCATGGTCAT | gray | Structure strand | 104 |
| | AAAAGGGG | | | |
| 21[120]23[127] | CCCAGCAGGCGAAAAATCCCTT | gray | Structure strand | 105 |
| | ATAAATCAAGCCGGCG | | | |
| 21[160]22[144] | TCAATATCGAACCTCAAATATC | gray | Structure strand | 106 |
| | AATTCCGAAA | | | |
| 21[184]23[191] | TCAACAGTTGAAAGGAGCAAA | gray | Structure strand | 107 |
| | TGAAAAATCTAGAGATAGA | | | |
| 21[224]23[223] | CTTTAGGGCCTGCAACAGTGCC | gray | Structure strand | 108 |
| | AATACGTG | | | |
| 21[248]23[255] | AGATTAGAGCCGTCAAAAAAC | gray | Structure strand | 109 |
| | AGAGGTGAGGCCTATTAGT | | | |
| 21[32]23[31] | TTTTCACTCAAAGGGCGAAAAA | gray | Structure strand | 110 |
| | CCATCACC | | | |
| 21[56]23[63] | AGCTGATTGCCCTTCAGAGTCC | gray | Structure strand | 111 |
| | ACTATTAAAGGGTGCCGT | | | |
| 21[96]23[95] | AGCAAGCGTAGGGTTGAGTGTT | gray | Structure strand | 112 |
| | GTAGGGAGCC | | | |
| 22[111]20[112] | GCCCGAGAGTCCACGCTGGTTT | gray | Structure strand | 113 |
| | GCAGCTAACT | | | |
| 22[143]21[159] | TCGGCAAATCCTGTTTGATGGT | gray | Structure strand | 114 |
| | GGACCCTCAA | | | |
| 22[175]20[176] | ACCTTGCTTGGTCAGTTGGCAA | gray | Structure strand | 115 |
| | AGAGCGGA | | | |
| 22[207]20[208] | AGCCAGCAATTGAGGAAGGTT | gray | Structure strand | 116 |
| | ATCATCATTTT | | | |
| 22[239]20[240] | TTAACACCAGCACTAACAACTA | gray | Structure strand | 117 |
| | ATCGTTATTA | | | |
| 22[271]20[272] | CAGAAGATTAGATAATACATTT | gray | Structure strand | 118 |
| | GTCGACAA | | | |
| 22[47]20[48] | CTCCAACGCAGTGAGACGGGC | gray | Structure strand | 119 |
| | AACCAGCTGCA | | | |
| 22[79]20[80] | TGGAACAACCGCCTGGCCCTGA | gray | Structure strand | 120 |
| | GGCCCGCT | | | |
| 23[128]23[159] | AACGTGGCGAGAAAGGAAGGG | gray | Structure strand | 121 |
| | AAACCAGTAA | | | |
| 23[160]22[176] | TAAAAGGGACATTCTGGCCAA | gray | Structure strand | 122 |
| | CAAAGCATC | | | |
| 23[192]22[208] | ACCCTTCTGACCTGAAAGCGTA | gray | Structure strand | 123 |
| | AGACGCTGAG | | | |
| 23[224]22[240] | GCACAGACAATATTTTTGAATG | gray | Structure strand | 124 |
| | GGGTCAGTA | | | |
| 23[256]22[272] | CTTTAATGCGCGAACTGATAGC | gray | Structure strand | 125 |
| | CCCACCAG | | | |
| 23[32]22[48] | CAAATCAAGTTTTTTGGGGTCG | gray | Structure strand | 126 |
| | AAACGTGGA | | | |
| 23[64]22[80] | AAAGCACTAAATCGGAACCCT | gray | Structure strand | 127 |
| | AATCCAGTT | | | |
| 23[96]22[112] | CCCGATTTAGAGCTTGACGGGG | gray | Structure strand | 128 |
| | AAAAAGAATA | | | |
| 3[160]4[144] | TTGACAGGCCACCACCAGAGC | gray | Structure strand | 129 |
| | CGCGATTTGTA | | | |
| 3[224]5[223] | TTAAAGCCAGAGCCGCCACCCT | gray | Structure strand | 130 |
| | CGACAGAA | | | |
| 3[32]5[31] | AATACGTTTGAAAGAGGACAG | gray | Structure strand | 131 |
| | ACTGACCTT | | | |
| 3[96]5[95] | ACACTCATCCATGTTACTTAGC | gray | Structure strand | 132 |
| | CGAAAGCTGC | | | |
| 4[111]2[112] | GACCTGCTCTTTGACCCCCAGC | gray | Structure strand | 133 |
| | GAGGGAGTTA | | | |
| 4[143]3[159] | TCATCGCCAACAAAGTACAAC | gray | Structure strand | 134 |
| | GGACGCCAGCA | | | |
| 4[175]2[176] | CACCAGAAAGGTTGAGGCAGG | gray | Structure strand | 135 |
| | TCATGAAAG | | | |
| 4[207]2[208] | CCACCCTCTATTCACAAACAAA | gray | Structure strand | 136 |
| | TACCTGCCTA | | | |
| 4[239]2[240] | GCCTCCCTCAGAATGGAAAGC | gray | Structure strand | 137 |
| | GCAGTAACAGT | | | |
| 4[271]2[272] | AAATCACCTTCCAGTAAGCGTC | gray | Structure strand | 138 |
| | AGTAATAA | | | |
| 4[47]2[48] | GACCAACTAATGCCACTACGA | gray | Structure strand | 139 |
| | AGGGGGTAGCA | | | |
| 4[79[2[80] | GCGCAGACAAGAGGCAAAAGA | gray | Structure strand | 140 |
| | ATCCCTCAG | | | |
| 5[160]6[144] | GCAAGGCCTCACCAGTAGCAC | gray | Structure strand | 141 |
| | CATGGGCTTGA | | | |
| 5[224]7[223] | TCAAGTTTCATTAAAGGTGAAT | gray | Structure strand | 142 |
| | ATAAAAGA | | | |
| 5[32]7[31] | CATCAAGTAAAACGAACTAAC | gray | Structure strand | 143 |
| | GAGTTGAGA | | | |
| 5[96]7[95] | TCATTCAGATGCGATTTTAAGA | gray | Structure strand | 144 |
| | ACAGGCATAG | | | |
| 6[111]4[112] | ATTACCTTTGAATAAGGCTTGC | gray | Structure strand | 145 |
| | CCAAATCCGC | | | |
| 6[143]5[159] | GATGGTTTGAACGAGTAGTAA | gray | Structure strand | 146 |
| | ATTTACCATTA | | | |
| 6[175]4[176] | CAGCAAAAGGAAACGTCACCA | gray | Structure strand | 147 |
| | ATGAGCCGC | | | |
| 6[207[4[208] | TCACCGACGCACCGTAATCAGT | gray | Structure strand | 148 |
| | AGCAGAACCG | | | |
| 6[239]4[240] | GAAATTATTGCCTTTAGCGTCA | gray | Structure strand | 149 |
| | GACCGGAACC | | | |
| 6[271]4[272] | ACCGATTGTCGGCATTTTCGGT | gray | Structure strand | 150 |
| | CATAATCA | | | |
| 6[47]4[48] | TACGTTAAAGTAATCTTGACAA | gray | Structure strand | 151 |
| | GAACCGAACT | | | |
| 6[79]4[80] | TTATACCACCAAATCAACGTAA | gray | Structure strand | 152 |
| | CGAACGAG | | | |
| 7[120]9[127] | CGTTTACCAGACGACAAAGAA | gray | Structure strand | 153 |
| | GTTTTGCCATAATTCGA | | | |
| 7[160]8[144] | TTATTACGAAGAACTGGCATGA | gray | Structure strand | 154 |
| | TTGCGAGAGG | | | |
| 7[184]9[191] | CGTAGAAAATACATACCGAGG | gray | Structure strand | 155 |
| | AAACGCAATAAGAAGCGCA | | | |
| 7[224]9[223] | AACGCAAAGATAGCCGAACAA | gray | Structure strand | 156 |
| | ACCCTGAAC | | | |
| 7[248]9[255] | GTTTATTTTGTCACAATCTTACC | gray | Structure strand | 157 |
| | GAAGCCCTTTAATATCA | | | |
| 7[32]9[31] | TTTAGGACAAATGCTTTAAACA | gray | Structure strand | 158 |
| | ATCAGGTC | | | |
| 7[56]9[63] | ATGCAGATACATAACGGGAAT | gray | Structure strand | 159 |
| | CGTCATAAATAAAGCAAAG | | | |
| 7[96]9[95] | TAAGAGCAAATGTTTAGACTGG | gray | Structure strand | 160 |
| | ATAGGAAGCC | | | |
| 8[111]6[112] | AATAGTAAACACTATCATAACC | gray | Structure strand | 161 |
| | CTCATTGTGA | | | |
| 8[143]7[159] | CTTTTGCAGATAAAAACCAAAA | gray | Structure strand | 162 |
| | TAAAGACTCC | | | |
| 8[175]6[176] | ATACCCAACAGTATGTTAGCAA | gray | Structure strand | 163 |
| | ATTAGAGC | | | |
| 8[207]6[208] | AAGGAAACATAAAGGTGGCAA | gray | Structure strand | 164 |
| | CATTATCACCG | | | |
| 8[239]6[240] | AAGTAAGCAGACACCACGGAA | gray | Structure strand | 165 |
| | TAATATTGACG | | | |
| 8[271]6[272] | AATAGCTATCAATAGAAAATTC | gray | Structure strand | 166 |
| | AACATTCA | | | |
| 8[47]6[48] | ATCCCCCTATACCACATTCAAC | gray | Structure strand | 167 |
| | TAGAAAAATC | | | |
| 8[79]6[80] | AATACTGCCCAAAAGGAATTA | gray | Structure strand | 168 |
| | CGTGGCTCA | | | |
| 9[128]11[127] | GCTTCAATCAGGATTAGAGAGT | gray | Structure strand | 169 |
| | TATTTTCA | | | |
| 9[160]10[144] | AGAGAGAAAAAAATGAAAATA | gray | Structure strand | 170 |
| | GCAAGCAAACT | | | |
| 9[192]11[191] | TTAGACGGCCAAATAAGAAAC | gray | Structure strand | 171 |
| | GATAGAAGGCT | | | |
| 9[224]11[223] | AAAGTCACAAAATAAACAGCC | gray | Structure strand | 172 |
| | AGCGTTTTA | | | |
| 9[256]11[255] | GAGAGATAGAGCGTCTTTCCAG | gray | Structure strand | 173 |
| | AGGTTTTGAA | | | |
| 9[32]11[31] | TTTACCCCAACATGTTTTAAAT | gray | Structure strand | 174 |
| | TTCCATAT | | | |
| 9[64]11[63] | CGGATTGCAGAGCTTAATTGCT | gray | Structure strand | 175 |
| | GAAACGAGTA | | | |
| 9[96]11[95] | CGAAAGACTTTGATAAGAGGT | gray | Structure strand | 176 |
| | CATATTTCGCA | | | |
| 4[63]6[56] | Biotin- | light | 5′-Biotin | 177 |
| | ATAAGGGAACCGGATATTCATT | gray | modification | |
| | ACGTCAGGACGTTGGGAA-3′ | | | |
| 4[127]6[120] | Biotin- | light | 5′-Biotin | 178 |
| | TTGTGTCGTGACGAGAAACACC | gray | modification | |
| | AAATTTCAACTTTAAT-3′ | | | |
| 4[191]6[184] | Biotin- | light | 5′-Biotin | 179 |
| | CACCCTCAGAAACCATCGATAG | gray | modification | |
| | CATTGAGCCATTTGGGAA-3′ | | | |
| 4[255]6[248] | Biotin- | light | 5′-Biotin | 180 |
| | AGCCACCACTGTAGCGCGTTTT | gray | modification | |
| | CAAGGGAGGGAAGGTAAA-3′ | | | |
| 18[63]20[56] | Biotin- | light | 5′-Biotin | 181 |
| | ATTAAGTTTACCGAGCTCGAAT | gray | modification | |
| | TCGGGAAACCTGTCGTGC-3′ | | | |
| 18[127]20[120] | Biotin- | light | 5′-Biotin | 182 |
| | GCGATCGGCAATTCCACACAAC | gray | modification | |
| | AGGTGCCTAATGAGTG-3′ | | | |
| 18[191]20[184] | Biotin- | light | 5′-Biotin | 183 |
| | ATTCATTTTTGTTTGGATTATAC | gray | modification | |
| | TAAGAAACCACCAGAAG-3′ | | | |
| 18[255]20[248] | Biotin- | light | 5′-Biotin | 184 |
| | AACAATAACGTAAAACAGAAA | gray | modification | |
| | TAAAAATCCTTTGCCCGAA-3′ | | | |

A “handle” and “anti-handle” strand strategy was used to attach dye molecules of interest to the molecular pegboard. At the position where a dye molecule is intended to be attached, the staple strands was extended with a ˜21 nucleotide long single-stranded handle sequence (see Table 2 for sequences). The complementary single-stranded anti-handle sequence was functionalized with the dye molecule intended to be attached to the DNA nanostructure (FIG. 1A). Staple strands attached to handle sequences and functionalized anti-handle strands are typically part of a one-pot assembly mix. Distinct target species can be attached to the origami pegboard by using orthogonal handle strand sequences (see, e.g., Lin, C. Nature chemistry 4, 832-839 (2012), incorporated by reference herein).

TABLE 2
Fluorescently-labeled single-stranded sequences.

| | SEQ | | SEQ |
|---|---|---|---|
| | ID | | ID |
| Label | NO: | Handle | NO: |
| 5′- | 186 | staple- | 189 |
| GTGATGTAGGTGGTAGAGGAA- | | TTCCTCTACCACCTACATCA | |
| Atto 647N | | C-3′ | |
| 5′- | 187 | staple- | 190 |
| TATGAGAAGTTAGGAATGTTA- | | TAACATTCCTAACTTCTCAT | |
| Cy3 | | A-3′ | |
| 5′- | 188 | staple- | 191 |
| CGAGTTTAGGAGAGATGGTAA- | | TTACCATCTCTCCTAAACTC | |
| Atto 488 | | G-3′ | |

### Tunable Brightness

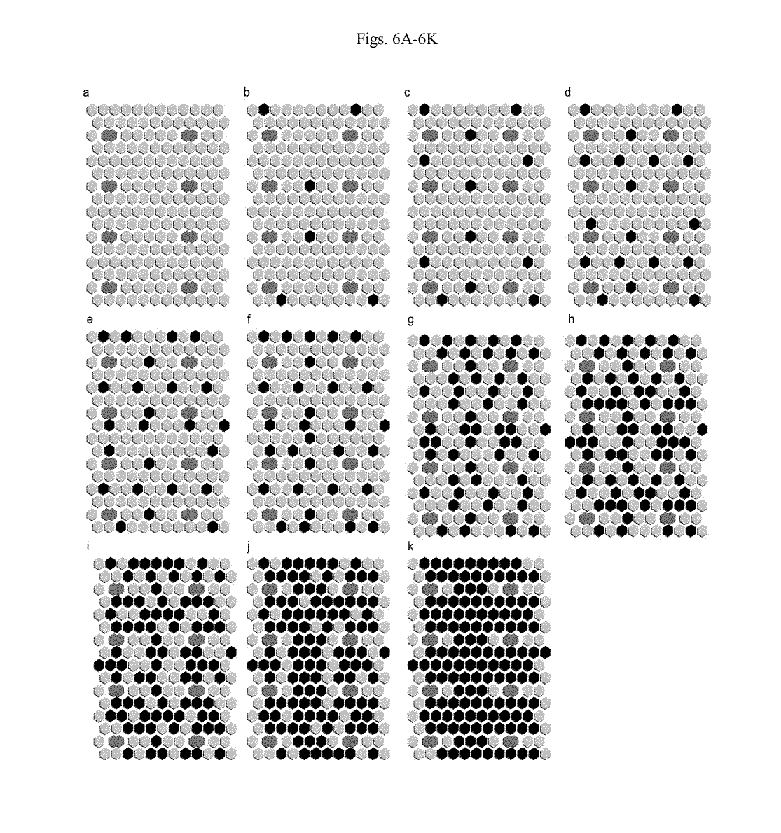

DNA nanostructures were designed with a prescribed number of dyes and dye molecules, ranging from 6 to 132 (FIG. 1A and FIG. 6). Each nanostructures species was assembled using a staple strand mix, which contained dye-labeled anti-handle and handle strands in a 2.25:1 molar ratio (see Materials and Methods). After self-assembly and purification, the metafluorophores (e.g., carrying 8 biotinylated capture strands) were immobilized on streptavidin coated glass slides in custom-made flow chambers (see Materials and Methods). Metafluorophores, in some instances, carry 1-200 biotinylated strands. In some instances, metafluorophores are immobilized in a different way. In some instances, antibodies and/or nanobodies, or aptamers, are attached to biotin and/or other binders.

Imaging was performed on a ˜100×100 μm² area containing ˜1000 DNA origami structures and single images were acquired for 10 seconds using LED illumination on an inverted epi-fluorescence microscope (see Materials and Methods). After image acquisition, a spot detection algorithm was used to identify individual DNA origami structures (appearing as bright spots in the fluorescence image). In a subsequent step, a 2-dimensional Gaussian fit was performed within a 10×10 px² area containing a spot. The volume under the Gaussian function was used as the measure of intensity.

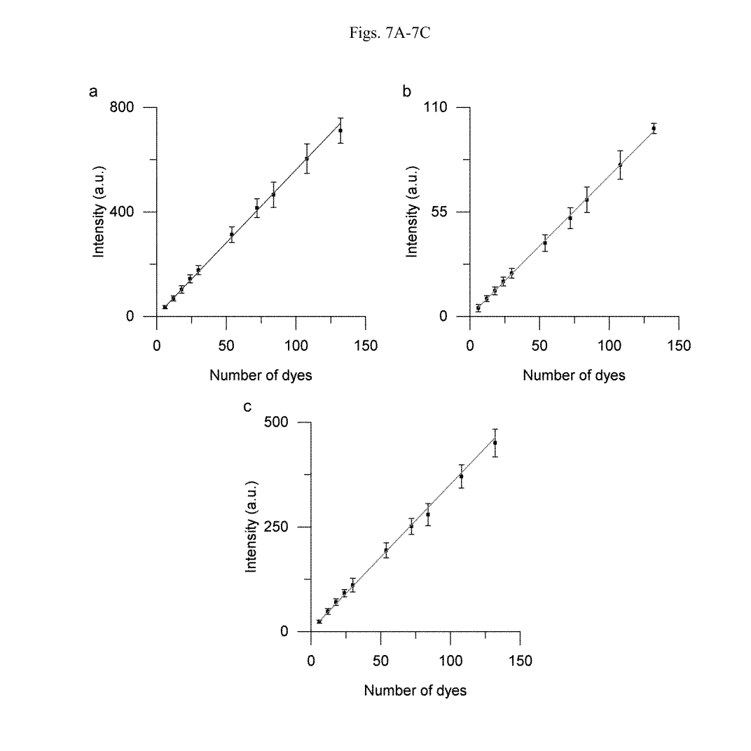

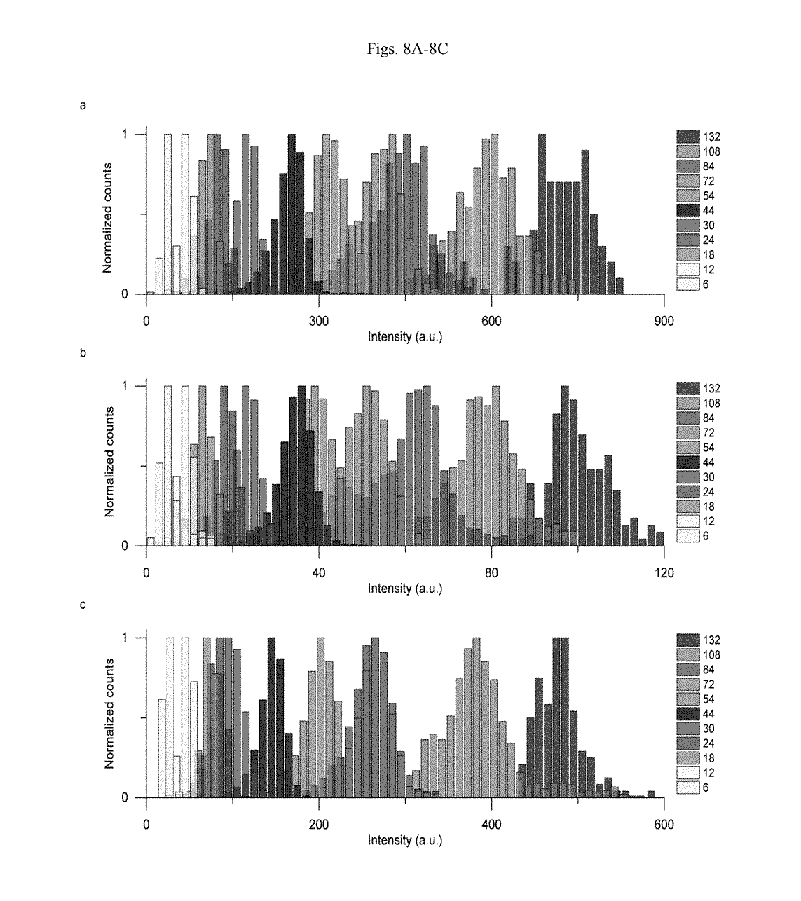

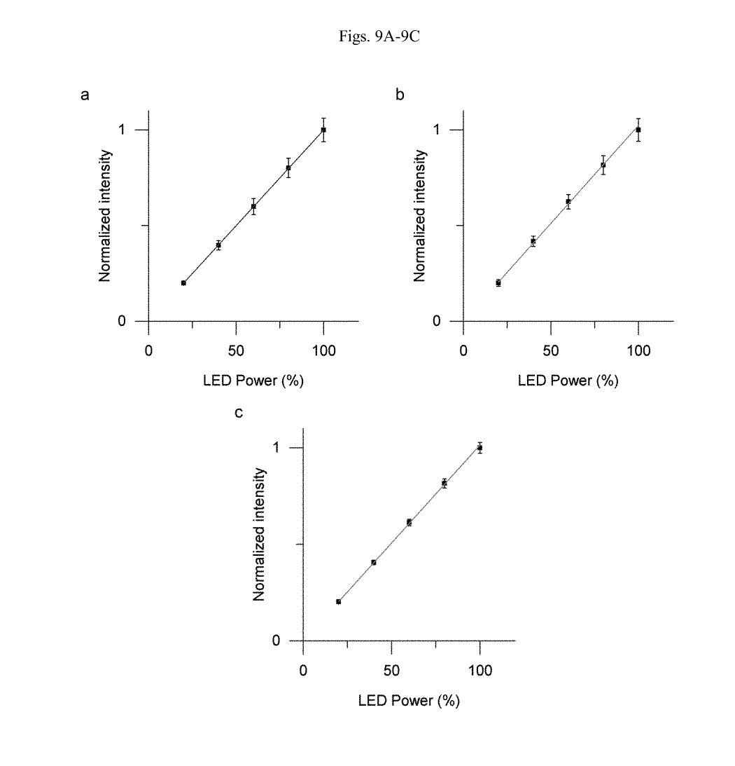

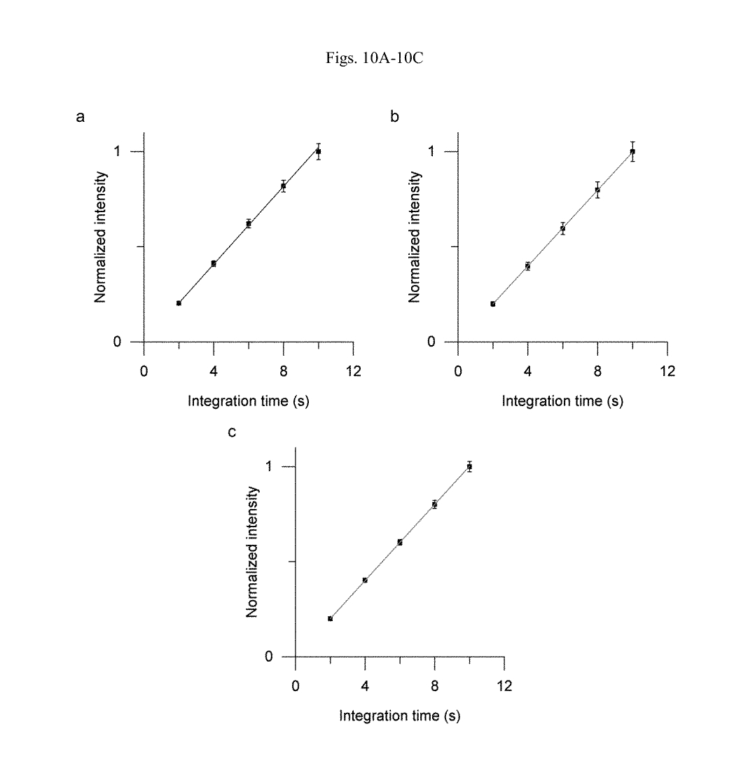

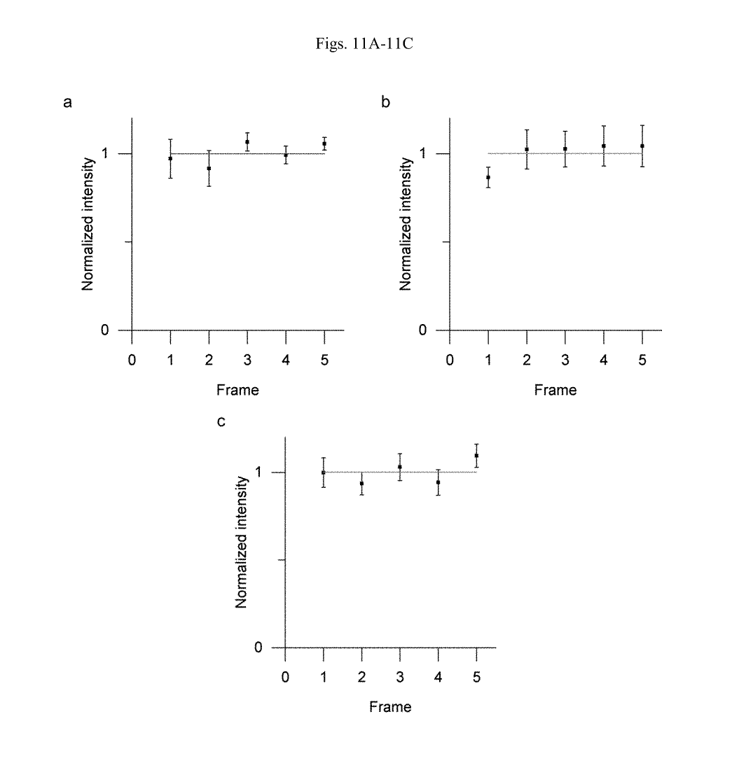

The metafluorophores showed a linear dependence of fluorescence intensity on the number of dyes within measurement accuracy. This linear dependence for Atto 647N, Cy3 and Atto 488 dyes was confirmed using metafluorophores carrying up to 132 dye molecules per structure (FIGS. 1B-1D, FIGS. 7A-7C and FIGS. 8A-8C). Dye molecules were spaced approximately equidistantly (see pictograms) on the nanostructures, and measurements for all species were performed independently analyzing ˜10,000 nanostructures. All measurements were performed after evaluating optimal acquisition settings (FIGS. 9A-9C and FIGS. 10A-10C).

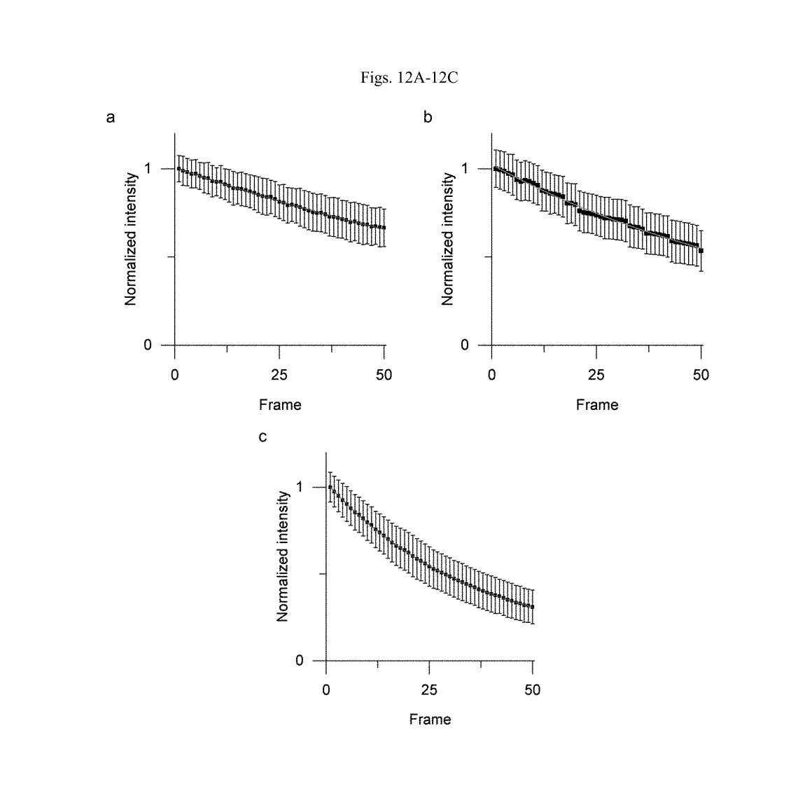

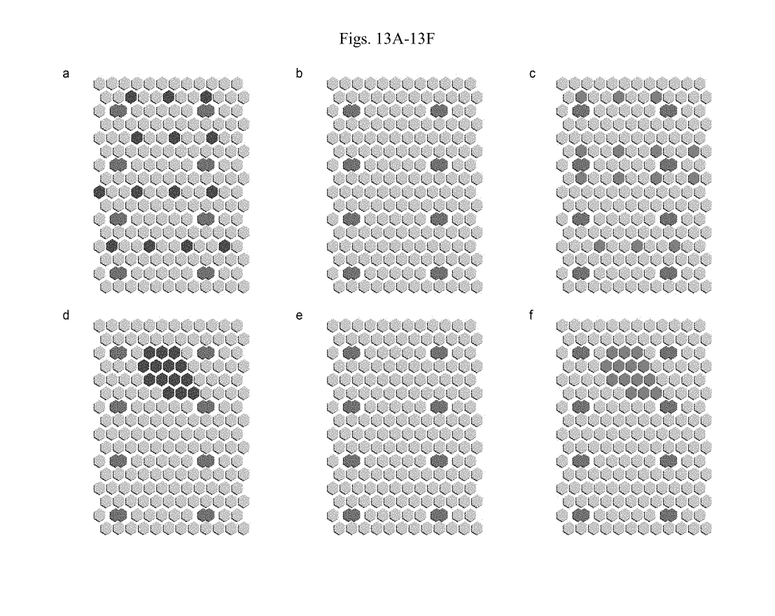

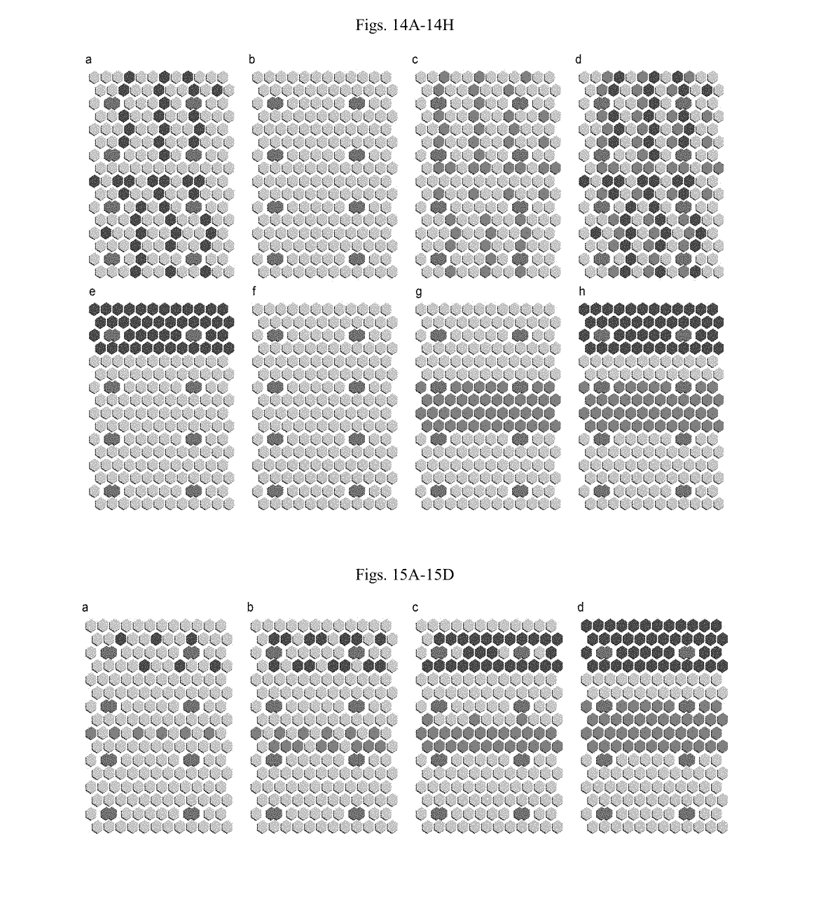

Intrinsic variations in measured fluorescence intensity are likely due to structure-to-structure variations on the number of dyes as well as stochastic properties of fluorescence emission of the dyes themselves. Extrinsic variations from sample-to-sample mainly originate from differences in image acquisition such as slightly different focal planes or photobleaching. If the fluorescence emission from a dye molecule is not acquired in perfect focus, fewer photons will be collected and thus the measured intensity will be decreased. To minimize this effect, an auto-focus system was used to maintain a constant focus. Repeated image acquisition of the same sample with intermittent refocusing yielded mean-to-mean variations of ˜5% (FIGS. 11C-11C). Additionally, each image acquisition “bleached” the samples by ˜0.8-2.8%, depending on the type of dye (FIGS. 12A-12C).

An important feature of a metafluorophore, in some instances, is its nanoscale size. This may become especially important when they are used to tag biomolecules in an in situ setting (e.g., inside a cell). In order to engineer and construct compact metafluorophores, dye molecules must be spaced close together while preventing unwanted dye-dye interactions such as self-quenching. To demonstrate that dye-dye interactions are actively prevented metafluorophores, experiments were performed with DNA nanostructures carrying (e.g., linked to) 14 dye molecules with low labeling density (˜16 nm dye-to-dye distance) and 14 dye molecules with high density (˜5 nm dye-to-dye distance), respectively, and compared their fluorescence intensity distributions (FIG. 1E-1G and FIGS. 13A-13F). Atto 647N-, Cy3- and Atto 488-labeled structures with low and high labeling density showed the same fluorescence intensities within measurement accuracy.

### Tunable Color

Metafluorophores were “functionalized,” as described above, with multiple orthogonal handle strands that can, in turn, bind spectrally distinct dye-labeled anti-handle strands. Next, structures labeled with either Atto 647N, Cy3, Atto 488, or a combination thereof were designed.

If spectrally distinct fluorophores are brought into close proximity (e.g., closer than ˜10 nm), they may exhibit Förster resonance energy transfer (FRET). In FRET, the fluorophore with the shorter excitation wavelength (donor) transfers energy to the fluorophore with the longer excitation wavelength (acceptor) through non-radiative dipol-dipol coupling. If FRET occurs, the donor dye's emission fluorescence intensity will be decreased, depending on the proximity and number of adjacent acceptor dyes.

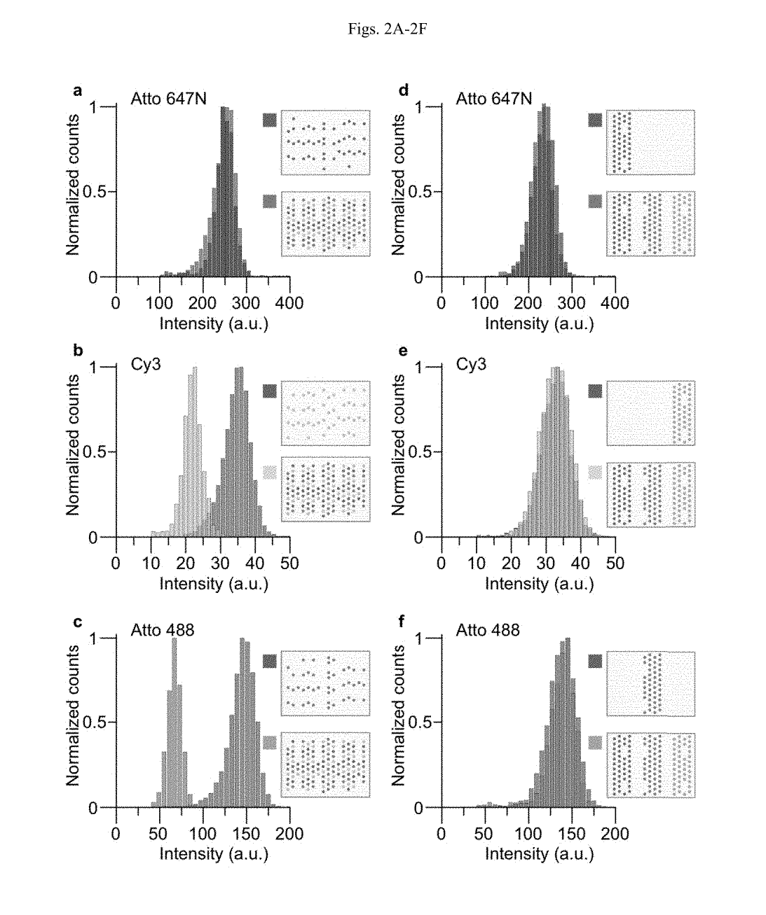

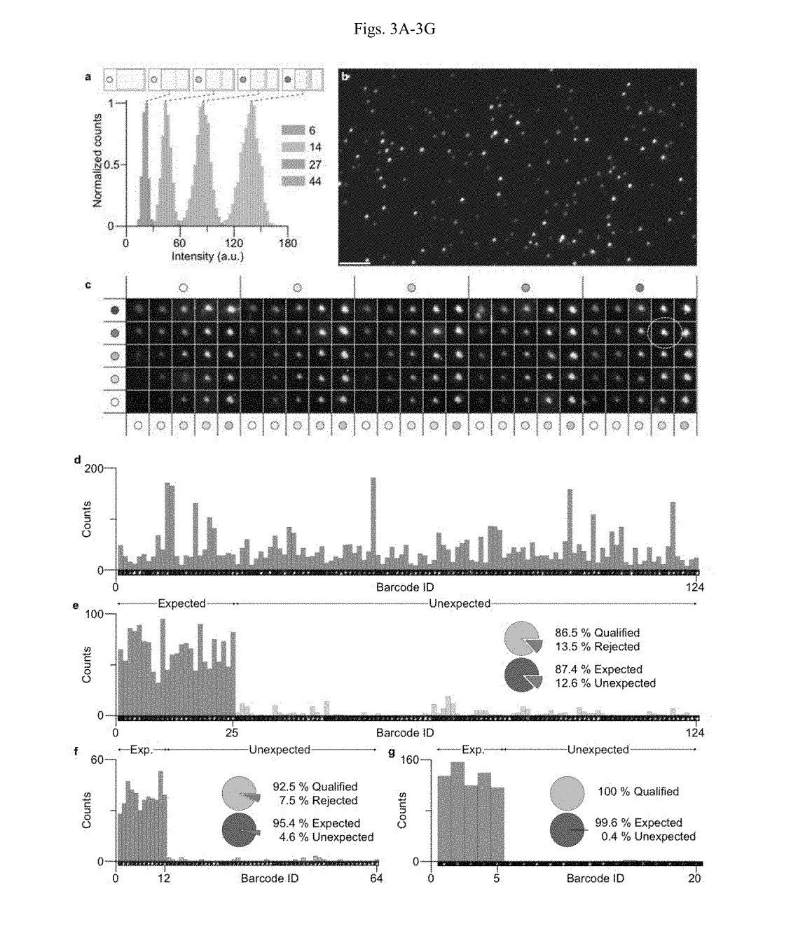

In order to maintain prescribed fluorescence intensities when using multiple fluorescent colors in metafluorophores, potential FRET between spectrally distinct dye molecules must be prevented. The following experiment investigates whether FRET occurs in metafluorophores, thus, limiting the capability to precisely design their fluorescence intensity and color. A metafluorophore design with 44 randomly arranged Atto 647N, Cy3, and Atto 488 dye molecules, respectively, were investigated (FIGS. 2A-2C and FIGS. 14A-14H). This random arrangement was tested by comparing two different metafluorophore: one contained all three dyes and one contained only a single fluorescent dye. The resulting intensity distributions suggest that Atto 488 and Cy3 act as FRET donors, as they exhibited a significant decrease in fluorescence intensity for the metafluorophore containing possible acceptor fluorophores. The mean intensities for Atto 488 and Cy3 dyes were reduced by 50% and 40%, respectively, relative to control species with only a single fluorescent color. However, the mean fluorescence intensity for Atto 647N was unchanged, as this dye lacks a potential FRET acceptor fluorophore.

The finding that FRET can indeed alter fluorescence emission intensity of the metafluorophore by as much as 50% in randomly labeled structures may limit the ability to control fluorescence color and intensity independently. However, the precise programmability of nucleic acid-based nanostructures, such as, for example, DNA origami, allows for an increase in the spacing of spectrally distinct dyes, thus preventing FRET while maintaining high labeling densities and nanoscale structure dimensions.

To improve the dye layout, a “column-like” arrangement of the three dye species was chosen to separate FRET donor and acceptor dyes into spatially distant zones (FIGS. 2D-F and FIGS. 15A-15D). Repeating the same experiments as represented in FIG. 2A-2C, fluorescence intensities between multi- and single-color species were unchanged, thus the modified “column-like” layout prevents FRET (FIG. 2D-2F). This permits the independent tuning of brightness and color of metafluorophores.

### Example 2

### Multiplexed Tagging

Having established the ability to precisely engineer photophysical properties, such as intensity and color, potential applications of metafluorophores were next investigated. In particular, the performance of metafluorophores as multiplexed labels based on intensity and color combinations was investigated. An important features of programmable metafluorophores is their usefulness as labeling probes for highly multiplexed target detection.

Due to stochastic photon emission, imperfect labeling and incomplete staple incorporation, metafluorophores show a finite intensity distribution for a defined number of dye molecules (FIG. 3A). If the intensity distributions of two distinct barcode levels (or numbers of dye molecules per structure) are engineered to have no overlap, each measured intensity value can be unambiguously assigned to a specific barcode.

The number of distinct barcode species N scales as N=a^{b}, with b being the number of spectrally distinct colors, and a the number of distinguishable intensity levels per color.

With a maximum number of 132 staple strands available for modification and three distinct dyes, the largest number of dye molecules per color per structure is 132/3=44. The smallest number of dye molecules that can be robustly detected using the standard inverted fluorescence microscope is ˜6.

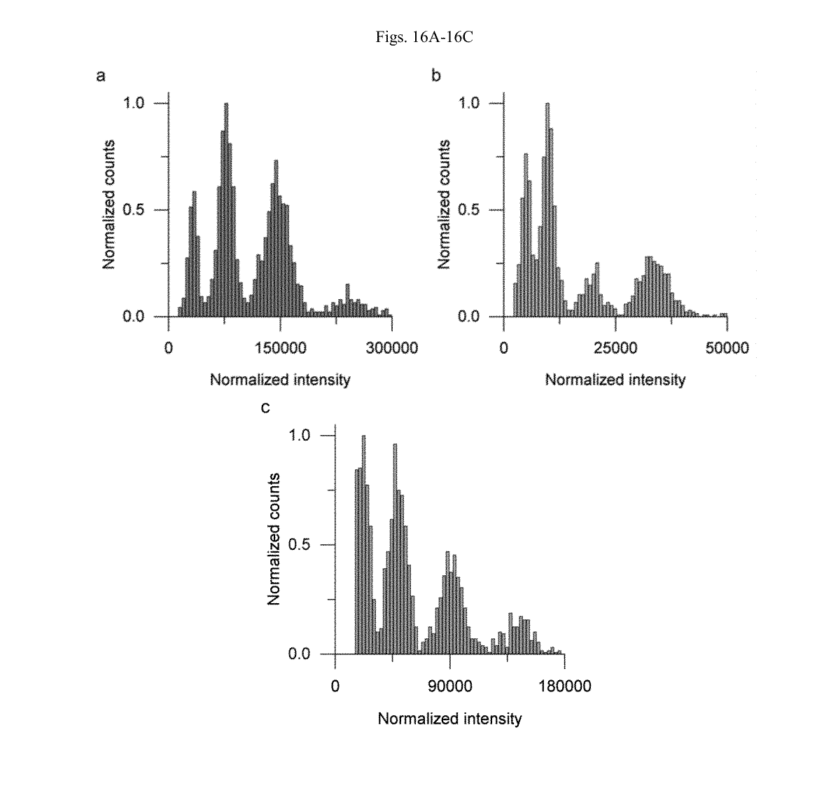

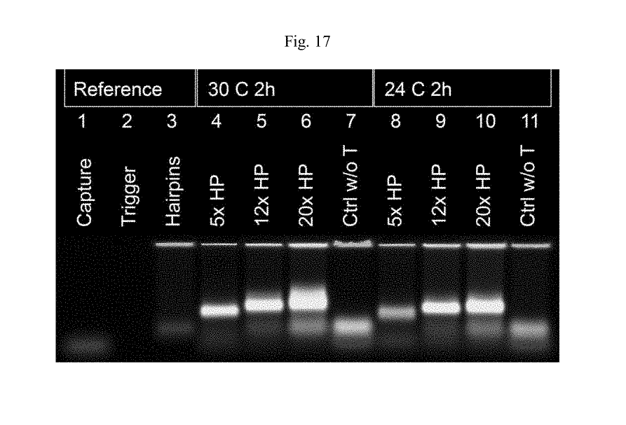

By measuring the width of the intensity distribution for different numbers of dyes on a metafluorophore, a total of four non-overlapping levels for use in a barcoding application were identified, corresponding to 6, 14, 27 and 44 dye molecules, respectively (FIGS. 16A-16C). Combinatorial labeling with three spectrally distinct dyes and five intensity levels (including 0) permits a maximum of 5³−1=124 barcodes with the example design presented here.

First, the ability to design, fabricate, and robustly identify all 124 possible barcodes was tested. After self-assembly and purification of the barcodes, the barcodes were pooled and immobilized in a streptavidin-modified flow chamber (FIGS. 3B and 3C). Image acquisition was performed sequentially, starting with the longest wavelength and subsequently imaging the shorter wavelengths to minimize photobleaching. Data analyses (e.g., spot detection and intensity measurement) were performed, as described above, for each color channel separately. During image analysis, each detected spot (and thus barcode) was assigned a coordinate and corresponding intensity value for each color. Co-localizing spots were combined and assigned to the same metafluorophore.

To identify the metafluorophore with a specific barcode identification, the measured intensity values were compared to a reference table in order to assign the correct barcode level. A new reference table for each sample acquisition can be obtained by creating a histogram of all measured intensity values (FIGS. 16A-16C). This has the benefit of a “real-time” check for sample performance. The overlap of adjacent distributions is an important measure for barcoding performance, as it represents intensity levels that cannot be assigned unambiguously to a specific barcode level. To quantify this overlap and discard corresponding barcodes, a Gaussian function was fitted to each intensity distribution. The intersection points of adjacent Gaussians were calculated and subsequently used to determine regions of overlap.

The ability to fabricate and identify all possible 124 barcodes in one sample is illustrated in FIG. 3D. Variations in barcode counts are due to different nanostructure concentrations, likely introduced in their folding and purification process.

In order to benchmark the barcoding performance of the metafluorophores, subsets of barcodes were studied and the following measures introduced. From all detected metafluorophores, those with valid intensity values (e.g., outside levels of overlapping intensity distributions) were qualified barcodes. As barcode subsets are measured, these qualified barcodes may consist of two sub-populations: expected (or correct) barcodes and unexpected (or false positive) barcodes. Consequently, a signal-to-noise-ratio (or SNR) was defined as (expected)/(unexpected). Together, these measures determined the overall performance of the barcoding system.

The first subset contained 25 randomly selected barcodes (FIG. 3E and Table 3). 2,155 spots were measured, of which 13.5% were discarded as unqualified barcodes with intensity values within overlapping regions. The discarded spots include misfolded structures as well as spots comprising multiple barcodes (e.g., spaced closer than the spatial resolution of the imaging system). For this 25-barcode subset, 87.4% of the qualified barcodes were expected. Here, an SNR of 27 was determined. A substantial population of false positives were single-colored barcodes with low fluorescence intensities (e.g., identified as “6-0-0”, “0-6-0” or “0-0-6”). Without being bound by theory, this may be an artifact arising from fluorescent surface impurities.

If the maximum multiplexing capacity is not required, more robust barcode sets with higher performance can be designed. This can be achieved by reducing the number of intensity levels and thereby spacing them further apart and consequently reducing overlapping intensity distributions. Additionally, using only three-colored barcodes makes detection and identification more robust (e.g., allows the rejection of single- and double-colored spots).

A total number of 64 three-color barcodes can be constructed using a metafluorophore design, for example. These barcodes were benchmarked by acquiring a subset of 12 structures (FIG. 3F and Table 3). Here, 512 spots were detected, 92.5% were qualified barcodes of which 95.4% were the expected ones. The SNR was determined to be 90.

TABLE 3
25/124 intensity barcode subset

| Barcode-No | RED | GRN | BLU | Subset No |
|---|---|---|---|---|
| 1 | 6 | 0 | 0 | |
| 2 | 14 | 0 | 0 | |
| 3 | 27 | 0 | 0 | |
| 4 | 44 | 0 | 0 | |
| 5 | 0 | 6 | 0 | |
| 6 | 6 | 6 | 0 | 1 |
| 7 | 14 | 6 | 0 | |
| 8 | 27 | 6 | 0 | |
| 9 | 44 | 6 | 0 | |
| 10 | 0 | 14 | 0 | |
| 11 | 6 | 14 | 0 | |
| 12 | 14 | 14 | 0 | |
| 13 | 27 | 14 | 0 | 2 |
| 14 | 44 | 14 | 0 | |
| 15 | 0 | 27 | 0 | |
| 16 | 6 | 27 | 0 | 3 |
| 17 | 14 | 27 | 0 | |
| 18 | 27 | 27 | 0 | |
| 19 | 44 | 27 | 0 | |
| 20 | 0 | 44 | 0 | |
| 21 | 6 | 44 | 0 | |
| 22 | 14 | 44 | 0 | |
| 23 | 27 | 44 | 0 | 4 |
| 24 | 44 | 44 | 0 | 5 |
| 25 | 0 | 0 | 6 | |
| 26 | 6 | 0 | 6 | |
| 27 | 14 | 0 | 6 | |
| 28 | 27 | 0 | 6 | 6 |
| 29 | 44 | 0 | 6 | 7 |
| 30 | 0 | 6 | 6 | |
| 31 | 6 | 6 | 6 | |
| 32 | 14 | 6 | 6 | |
| 33 | 27 | 6 | 6 | |
| 34 | 44 | 6 | 6 | |
| 35 | 0 | 14 | 6 | |
| 36 | 6 | 14 | 6 | |
| 37 | 14 | 14 | 6 | |
| 38 | 27 | 14 | 6 | |
| 39 | 44 | 14 | 6 | |
| 40 | 0 | 27 | 6 | |
| 41 | 6 | 27 | 6 | |
| 42 | 14 | 27 | 6 | |
| 43 | 27 | 27 | 6 | |
| 44 | 44 | 27 | 6 | |
| 45 | 0 | 44 | 6 | |
| 46 | 6 | 44 | 6 | |
| 47 | 14 | 44 | 6 | |
| 48 | 27 | 44 | 6 | |
| 49 | 44 | 44 | 6 | |
| 50 | 0 | 0 | 14 | |
| 51 | 6 | 0 | 14 | |
| 52 | 14 | 0 | 14 | |
| 53 | 27 | 0 | 14 | |
| 54 | 44 | 0 | 14 | |
| 55 | 0 | 6 | 14 | |
| 56 | 6 | 6 | 14 | |
| 57 | 14 | 6 | 14 | |
| 58 | 27 | 6 | 14 | 8 |
| 59 | 44 | 6 | 14 | |
| 60 | 0 | 14 | 14 | 9 |
| 61 | 6 | 14 | 14 | 10 |
| 62 | 14 | 14 | 14 | |
| 63 | 27 | 14 | 14 | |
| 64 | 44 | 14 | 14 | 11 |
| 65 | 0 | 27 | 14 | |
| 66 | 6 | 27 | 14 | |
| 67 | 14 | 27 | 14 | 12 |
| 68 | 27 | 27 | 14 | |
| 69 | 44 | 27 | 14 | 13 |
| 70 | 0 | 44 | 14 | |
| 71 | 6 | 44 | 14 | |
| 72 | 14 | 44 | 14 | |
| 73 | 27 | 44 | 14 | 14 |
| 74 | 44 | 44 | 14 | 15 |
| 75 | 0 | 0 | 27 | |
| 76 | 6 | 0 | 27 | |
| 77 | 14 | 0 | 27 | |
| 78 | 27 | 0 | 27 | |
| 79 | 44 | 0 | 27 | 16 |
| 80 | 0 | 6 | 27 | |
| 81 | 6 | 6 | 27 | |
| 82 | 14 | 6 | 27 | 17 |
| 83 | 27 | 6 | 27 | |
| 84 | 44 | 6 | 27 | |
| 85 | 0 | 14 | 27 | |
| 86 | 6 | 14 | 27 | |
| 87 | 14 | 14 | 27 | 18 |
| 88 | 27 | 14 | 27 | |
| 89 | 44 | 14 | 27 | 19 |
| 90 | 0 | 27 | 27 | 20 |
| 91 | 6 | 27 | 27 | |
| 92 | 14 | 27 | 27 | |
| 93 | 27 | 27 | 27 | |
| 94 | 44 | 27 | 27 | |
| 95 | 0 | 44 | 27 | |
| 96 | 6 | 44 | 27 | |
| 97 | 14 | 44 | 27 | 21 |
| 98 | 27 | 44 | 27 | |
| 99 | 44 | 44 | 27 | |
| 100 | 0 | 0 | 44 | |
| 101 | 6 | 0 | 44 | |
| 102 | 14 | 0 | 44 | |
| 103 | 27 | 0 | 44 | 22 |
| 104 | 44 | 0 | 44 | |
| 105 | 0 | 6 | 44 | 23 |
| 106 | 6 | 6 | 44 | |
| 107 | 14 | 6 | 44 | |
| 108 | 27 | 6 | 44 | |
| 109 | 44 | 6 | 44 | |
| 110 | 0 | 14 | 44 | |
| 111 | 6 | 14 | 44 | |
| 112 | 14 | 14 | 44 | |
| 113 | 27 | 14 | 44 | |
| 114 | 44 | 14 | 44 | |
| 115 | 0 | 27 | 44 | |
| 116 | 6 | 27 | 44 | |
| 117 | 14 | 27 | 44 | |
| 118 | 27 | 27 | 44 | |
| 119 | 44 | 27 | 44 | |
| 120 | 0 | 44 | 44 | 24 |
| 121 | 6 | 44 | 44 | 25 |
| 122 | 14 | 44 | 44 | |
| 123 | 27 | 44 | 44 | |
| 124 | 44 | 44 | 44 | |

Even more robust barcodes can be constructed by excluding two barcode levels and spacing the remaining levels (e.g., 0, 14 and 44 dye molecules) further apart. Additionally barcodes contained at least two colors, thus a maximum of 20 distinguishable barcodes is achievable. A subset of 5 barcodes (N=664) were measured with a qualification ratio of 100%, e.g., all detected spots were positively identified as valid barcodes (FIG. 3G and Table 4). Here, only 3 false positives were counted, yielding 99.6% expected barcodes.

The false positives may be underestimated. It is possible to make a false identification of a spot without noticing, as the identified barcode may also be part of the used subset. Thereby, smaller subsets may yield higher identification accuracy.

TABLE 4
12/64 intensity barcode subset

| Barcode-No | RED | GRN | BLU | Subset No |
|---|---|---|---|---|
| 1 | 6 | 6 | 6 | |
| 2 | 14 | 6 | 6 | |
| 3 | 27 | 6 | 6 | |
| 4 | 44 | 6 | 6 | |
| 5 | 6 | 14 | 6 | 1 |
| 6 | 14 | 14 | 6 | |
| 7 | 27 | 14 | 6 | |
| 8 | 44 | 14 | 6 | |
| 9 | 6 | 27 | 6 | |
| 10 | 14 | 27 | 6 | |
| 11 | 27 | 27 | 6 | 2 |
| 12 | 44 | 27 | 6 | |
| 13 | 6 | 44 | 6 | |
| 14 | 14 | 44 | 6 | 3 |
| 15 | 27 | 44 | 6 | |
| 16 | 44 | 44 | 6 | |
| 17 | 6 | 6 | 14 | |
| 18 | 14 | 6 | 14 | |
| 19 | 27 | 6 | 14 | 4 |
| 20 | 44 | 6 | 14 | |
| 21 | 6 | 14 | 14 | |
| 22 | 14 | 14 | 14 | 5 |
| 23 | 27 | 14 | 14 | |
| 24 | 44 | 14 | 14 | 6 |
| 25 | 6 | 27 | 14 | |
| 26 | 14 | 27 | 14 | |
| 27 | 27 | 27 | 14 | |
| 28 | 44 | 27 | 14 | |
| 29 | 6 | 44 | 14 | |
| 30 | 14 | 44 | 14 | |
| 31 | 27 | 44 | 14 | |
| 32 | 44 | 44 | 14 | |
| 33 | 6 | 6 | 27 | |
| 34 | 14 | 6 | 27 | |
| 35 | 27 | 6 | 27 | |
| 36 | 44 | 6 | 27 | |
| 37 | 6 | 14 | 27 | |
| 38 | 14 | 14 | 27 | |
| 39 | 27 | 14 | 27 | |
| 40 | 44 | 14 | 27 | |
| 41 | 6 | 27 | 27 | |
| 42 | 14 | 27 | 27 | |
| 43 | 27 | 27 | 27 | 7 |
| 44 | 44 | 27 | 27 | |
| 45 | 6 | 44 | 27 | 8 |
| 46 | 14 | 44 | 27 | |
| 47 | 27 | 44 | 27 | |
| 48 | 44 | 44 | 27 | 9 |
| 49 | 6 | 6 | 44 | |
| 50 | 14 | 6 | 44 | |
| 51 | 27 | 6 | 44 | |
| 52 | 44 | 6 | 44 | |
| 53 | 6 | 14 | 44 | |
| 54 | 14 | 14 | 44 | |
| 55 | 27 | 14 | 44 | |
| 56 | 44 | 14 | 44 | 10 |
| 57 | 6 | 27 | 44 | |
| 58 | 14 | 27 | 44 | 11 |
| 59 | 27 | 27 | 44 | |
| 60 | 44 | 27 | 44 | |
| 61 | 6 | 44 | 44 | 12 |
| 62 | 14 | 44 | 44 | |
| 63 | 27 | 44 | 44 | |
| 64 | 44 | 44 | 44 | |

TABLE 5
5/20 intensity barcode subset

| Barcode-No | RED | GRN | BLU | Subset No |
|---|---|---|---|---|
| 1 | 44 | 14 | 14 | |
| 2 | 14 | 44 | 14 | 1 |
| 3 | 14 | 14 | 44 | |
| 4 | 44 | 44 | 14 | |
| 5 | 44 | 14 | 44 | 2 |
| 6 | 14 | 44 | 44 | |
| 7 | 44 | 44 | 44 | |
| 8 | 14 | 14 | 14 | |
| 9 | 44 | 44 | 0 | |
| 10 | 44 | 0 | 44 | 3 |
| 11 | 0 | 44 | 44 | |
| 12 | 14 | 14 | 0 | |
| 13 | 14 | 0 | 14 | |
| 14 | 0 | 14 | 14 | 4 |
| 15 | 44 | 14 | 0 | |
| 16 | 44 | 0 | 14 | |
| 17 | 0 | 44 | 14 | |
| 18 | 14 | 44 | 0 | 5 |
| 19 | 14 | 0 | 44 | |
| 20 | 0 | 14 | 44 | |

Nucleic acid-based self-assembled nanostructures, referred to as metafluorophores, can be considered a new kind of dye having digitally tunable optical properties, being hundreds of times brighter with arbitrarily prescribed intensity levels, and possessing digitally tunable “color”. The results presented herein demonstrate high labeling density (˜5 nm dye-to-dye distance) of nucleic acid-based nanostructures while preventing self-quenching. Further, the precise spatial control over dye positions on the nanostructures permits construction of nanoscale multicolor metafluorophores, where FRET between spectrally distinct dyes is prevented.

Combining these programmable features, 124 unique intensity barcodes were constructed for high content imaging. The feasibility of this approach was demonstrated, the in vitro performance was benchmarked, and the high specificity, identification accuracy and low false positive rate were shown.

Beyond surface-based microscopy applications, the combination of high brightness, small size and high multiplexing capacity of the metafluorophores render them ideal probes for applications such as, for example, flow cytometry and fluorescence correlation spectroscopy (FCS) for high throughput identification. The metafluorophores of the present disclosure may be extended to even smaller-sized structures by using the recently developed single-stranded tile assembly approach (Wei, B., et al. Nature 485, 623-626 (2012); Ke, Y., et al. Science 338, 1177-1183 (2012); Myhrvold, C., et al. Nano letters 13, 4242-4248 (2013), incorporated by reference herein). Further, metafluorophores can readily enhance signal intensity and multiplexing for use in current super-resolution techniques⁵⁶, such as (non-linear) structured illumination microscopy (SIM)⁵⁷.

Finally, metafluorophores based on triggered-assembly may be particularly useful for improving signal-to-noise and labeling efficiency in quantitative single-molecule FISH applications.

### Example 3

### Triggered Assembly

Moving from a “clean” in vitro environment to in situ cellular labeling applications poses additional challenges. Although multicolor metafluorophores made from ex situ self-assembled nucleic acid structures are nanoscale in size, they are still considerably larger than single small molecule labels, such as single dyes or nucleic acid strands. As this is still acceptable for in vitro applications (here mainly limiting reaction kinetics due to diffusion speed of the extended structures), it potentially has a major impact in in situ applications such as labeling of small proteins or nucleic acids in a densely crowded cellular environment. Additionally, possible non-specific interactions of preassembled barcodes with cellular components could lead to false positives.

To overcome both the challenge, provided herein is a method of triggering self-assembly of metafluorophores upon target detection. Short fluorescently-labeled, metastable hairpins were used, which assemble into a finite triangular structure only if a target molecule acting as trigger is present (FIG. 4A and Table 6). The in vitro triggered assembly of a defined-size (10 dyes) triangular metafluorophore using a trigger strand was demonstrated, immobilized by a dye-labeled capture strand on a glass surface.

First, an Alexa 647-labeled and biotinylated capture strand and the trigger strand were annealed and immobilized on a BSA-Biotin-Streptavidin-coated glass surface. Second, Cy3-labeled metastable hairpins were flowed in and incubated for 60 min. Lastly, DNA nanostructure-based metafluorophores carrying 44 Atto 488 and 10 Cy3-labeled strands were bound to the surface, as an intensity reference.

Image acquisition was performed by sequentially recording the Alexa 647, Cy3, Atto 488 channels (FIG. 4B). Co-localizations in the Alexa 647 and Cy3 channel represent the triangles, while Atto 488 and Cy3 co-localizations represent the nanostructure references.

To benchmark the formation performance of the triangles, the intensities of the origami reference structures were compared with the intensities of the triangles in the Cy3 channel (FIG. 4C). Gaussian fits to both intensity distributions revealed an almost perfect overlap with a mean-to-mean variation of less than 2%, suggesting the expected triangle formation. Both the formation of the triangle in the presence of the trigger and the meta-stability of the hairpins in the absence of the trigger were further confirmed by a formation gel assay.

The triggered metafluorophore assembly approach as provided herein has several advantages relative to existing assembly methods. Compared to single molecule fluorescent in situ hybridization (smFISH), for example, the programmability of the metafluorophores permits the assembly of more complex structures at the target site by, for example, using a transducer (initiator) molecule that is used to program complex structure assembly on-site. Unlike Hybridization Chain Reaction scheme (HCR) that produces a linear polymer structure of unspecified length, a structure of precisely defined size and shape is formed using the triggered assembly method as provided herein. Additionally, unlike previous methods of assembly of defined size structures that use a large number of unique monomer species, the methods herein, in some embodiments, use only one monomer species and the final size and shape of the metafluorophore is controlled by the length of the trigger strand. Compared to hybridization chain reaction (HCR), for example, the defined size and thus controlled intensity of the metafluorophore leads to higher multiplexing capability.

TABLE 6
Triggered assembly sequences.

| Description | Sequence | SEQ ID NO: |
|---|---|---|
| Capture | Alexa 647-CTCCTCGCCCTTGCTCACCAT-Biotin | 192 |
| Trigger | 5′-ATGGTGAGCAAGGGCGAGGAG... | 193,194, 194 |
| | CCTCACCTCTACTCCCACCCACACGCACCCTC | |
| | CCTCACCTCTACTCCCACCCACACGCACCCTC | |
| | ... | |
| | CCTCACCTCTACTCCCACCCACACGCACCCTC | |
| | CCTCACCTCTACTCCCACCCACACGCACCCTC- | |
| | 3′ | |
| Hairpin | Cy3-TCCCACCCACACGCACCCTC | 195, 196 |
| | CCTCACCTCTAC... | |
| | GAGGGTGCGTGTGGGTGGGA GTAGAGGTGAGG- | |
| | 3′ | |

### Example 4

### Ultra-Sensitive, Quantitative and Multiplexed Nucleic Acid Detection.

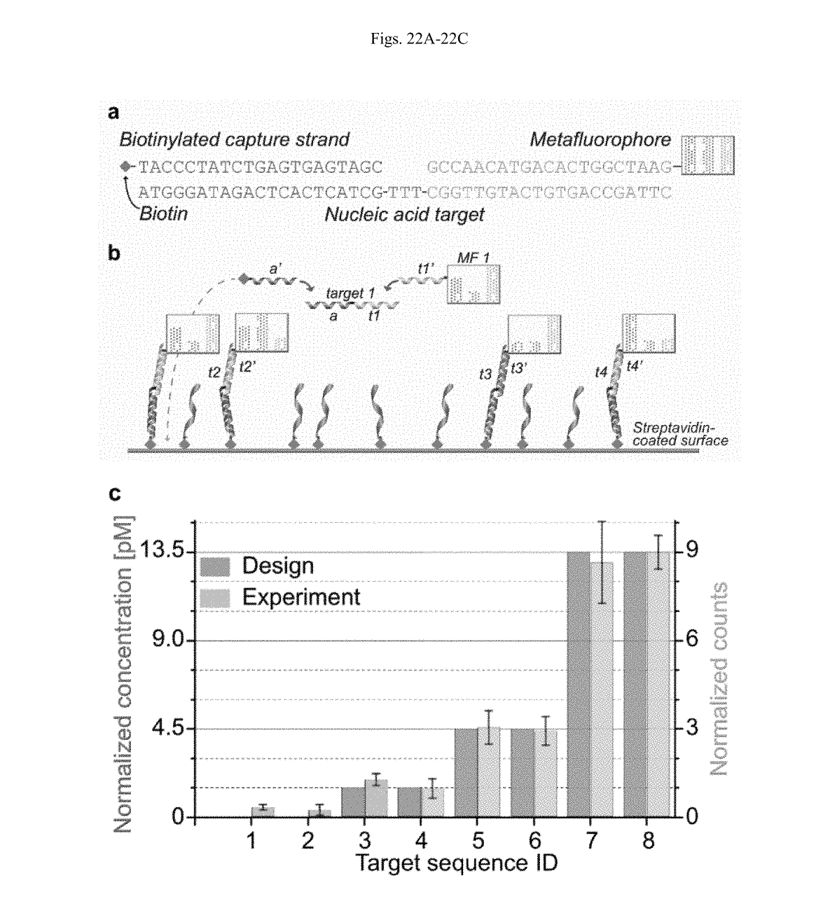

Implementing the metafluorophores in a multiplexed in vitro nucleic acid detection assay is described below. Each nucleic acid target (here eight synthetic DNA strands) is associated with a metafluorophore. The chosen metafluorophores are programed to specifically bind the target by replacing the eight biotinylated staples (previously used to attach the metafluorophore to the surface) with eight staples that are extended with a target complementary 21 nt long sequence at the 5′ end. To detect target-metafluorophore duplexes on a microscopy slide—comparable to the experiments of FIGS. 3A-3F—a biotinylated DNA strand (‘capture strand’) complementary to a second 21 nt region on the target is introduced (see FIGS. 22A and 22B).The three components are combined in a hybridization buffer and incubated for 24 h (see Materials and Methods). Concentrations of 1 nM biotinylated capture strands and approx. 250 pM metafluorophores per target were used. Targets were added in different amounts to demonstrate precise quantification and sensitivity (FIG. 22C). After incubation, the mixture was added into streptavidin coated flow chambers as before and incubated for 10 min. The chamber was subsequently washed and sealed. A scanning confocal microscope was used for data acquisition to demonstrate that the metafluorophores can be independently identified in a robust fashion.

To assess how precise and how sensitive this nucleic acid detection platform is, eight capture-target-metafluorophore triplets were designed and different amounts of six targets were added to the reaction. The remaining two targets were not added and, thus, indicate false-positives as before. The number of detected triplets is directly proportional to the initial target concentration and the targets can thus be relatively quantified. FIG. 22C shows the successful detection and precise quantification of targets with initial concentrations of 13.5 pM, 4.5 pM and 1.5 pM; the later corresponding to a target amount of only ˜100 fg. The number of counted metafluorophores has been corrected, using a calibration sample with equally concentrated targets, to minimize effects of discrepant initial concentrations.

### Example 5

### Additional Programmable Metafluorophore Properties.

Beyond brightness and color, additional dye properties can be used to expand the programmability of the metafluorophores. This is done by the controlled modification of the metafluorophores with groups of fluorescent molecules displaying the desired property. Suitable dye properties include, for example, fluorescence lifetime, the ability to photoactivate and switch, as well as photostability. These parameters can be tuned independently, similar to brightness and color, thus presenting additional orthogonal axes of programmability. This is especially valuable for multiplexed tagging, because the number of unambiguous labels scales with the power of independent parameters.

Here, differentiation and identification of metafluorophores based on the photostability of dyes was demonstrate the. Metafluorophores that contain two dyes with similar emission spectra, but different photostability under our imaging conditions were designed. Atto 647N was chosen as a dye with slower bleaching constant (more photostable) and Alexa647 as a dye with faster bleaching constant (less photostable). In a time-lapsed image acquisition experiment, the metafluorophores containing Alexa647 dyes bleach faster than the ones with Atto 647N dyes. As the fluorescence intensity decreases exponentially, the decay constant was measured, which was then used as parameter for photostability.

FIG. 21A shows a time-lapsed series of images of the two types of metafluorophores in one sample, where one species bleached faster than the other. Metafluorophores that contain multiple orthogonal properties can be identified in a multidimensional graph (FIG. 21B). For example, the bleaching (or decay) constant vs. the fluorescence intensity can be plotted. Distinct populations corresponding to different metafluorohpore configurations can be easily separated and identified (FIG. 21B). A one-dimensional histogram of the decay constants (FIG. 21C) clearly demonstrates that the photostability can be used as an orthogonal tunable metafluorophore property, similar to intensity discussed above.

### Example 6

Intensity Barcoding is a powerful tool for multiplexing applications in fluorescence microscopy. However, the total amount of barcodes is limited by availability of spectrally-distinct colors. To address this limitation, additional bleaching-kinetic based ‘virtual’ colors are introduced. Bleaching further enables the usage of FRET to encode dye arrangement in an intensity signature, increasing the multiplexing capability further. Usage of bleaching kinetics as an additional barcoding axis is directly applicable to intensity barcodes.

### Bleaching Barcodes

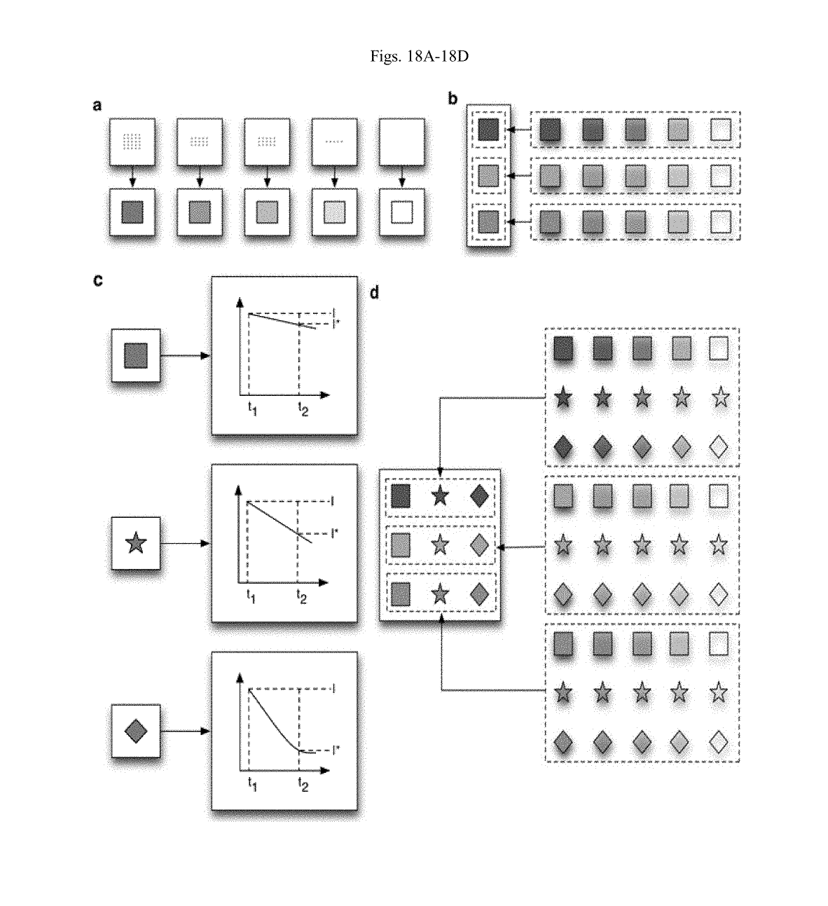

Intensity barcodes may be constructed by varying of the amount of fluorophores bound to a DNA nanostructures. As the schematics in FIG. 18A suggest, defined numbers of fluorophores create different intensity levels, so that measured intensities can be attributed to one population only. This is the case when no overlap between neighboring intensity levels exists. Introducing spectrally distinct dyes and combinatorial labeling of intensity levels, it is possible to create a vast amount of distinguishable structures. The number of possible Barcodes |X| calculates as all combinations of colors C and possible intensity levels I for each color (FIG. 18B).

|X|=I^{C}

For three colors and four intensity levels per color, it is possible to construct 4³=64 barcodes. To increase the number of barcodes, ‘virtual colors” based on bleaching kinetics are created.

### Fluorophore Bleaching

Fluorophores can be chemically destructed in their excited energy state while undergoing fluorescence emission. They consequently lose their ability to fluoresce: they become photobleached. As this decay in fluorescence is dye specific, one can characterize different dye types by their bleaching rate. These rates can be determined when recording and averaging the time-dependent-fluorescence intensity of multiple fluorophores. As bleaching is a stochastic process, it is impossible to assign a precise bleaching rate through the observation of a single dye bleaching event.

It is possible to sum individual fluorescence intensities in one diffraction-limited spot by placing them in close proximity on a DNA nanostructure. The resulting time course is dye-specific and can be used to introduce additional “virtual” colors for barcoding purposes, as FIG. 18C suggests. Consequently, dyes can be spectrally overlapping but still be distinguished by their bleaching rate.

### FRET

The application of bleaching kinetics may also be used to utilize FRET interactions. As already observed, dye pairs in close proximity can be prone to FRET. Using “photobleaching” of dyes will make FRET time-dependent. It is thus possible to encode and decode geometrical information within a nanostructure while still only observing a diffraction-limited, “structureless” spot.

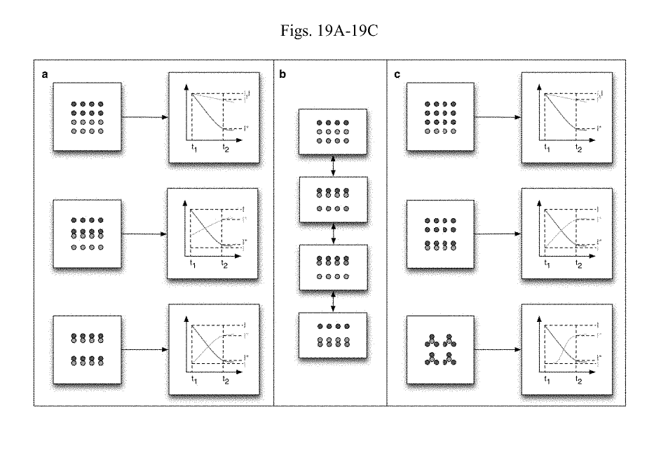

FIG. 19A shows possible arrangements of dye molecules on a nanostructure, depicting “pseudo-geometrical” coding. The expected FRET-signature depends on the number of dyes that have a FRET-partner. With increasing number of FRET pairs the signal in the donor channel will decrease, whereas the signal will be unchanged when no FRET pairs are existent. As FRET can occur between multiple colors, several overlapping arrangements are possible (FIG. 19B). When combining different group sizes (intensity levels) the number of possible arrangements can be further increased, as now a donor dye may have multiple acceptor dyes. FIG. 19C illustrates the variation of up to three acceptor dyes in close proximity to a donor dye when combining a low with a high intensity level. While the decay of the Alexa 647 dye (dark gray) stays the same for all the different structures, the time course of the intensity increase in the Cy3 (light gray) channel varies. For more acceptor partners there is a delay in the increase in the donor channel, as all of the acceptor dyes need to be bleached first. It is therefore possible to encode geometrical information in a structure and increase the amount of barcodes even further.

### Example 7

### Duplex-Barcodes for Detection of Small Targets

Intensity-based barcodes feature high multiplexing capacity without relying on spatial resolution, geometric information or time-lapse recordings. They are therefore ideal for the use in high throughput technologies, such as flow-cytometry, Fluorescence Correlation Spectroscopy (FCS) and wide-field microscopy, in general.

If the barcodes are not only required to identify, but also to detect target molecules, they must unambiguously indicate a positive detection of a target. For surface based detection or in situ studies, positive detection of a target is indicated by the presence of the barcode after a washing step. However, this does not apply for high throughput solution based techniques. Detection of a target in such instances should yield activation and/or switching of the barcode.

Such activation and/or switching may be achieved by triggering duplex formation of two barcodes upon detection of a target molecule. Detection of a barcode dimer in a sample solution, distinguished from a barcode monomer, indicates the presence of a target molecule in the solution under. By identifying the barcodes, the target species can be identified,

### Duplex Formation

The duplex formation mechanism depends on the target. Mechanisms described herein are based on nucleic acid detection.

If the target is a long (e.g., 30 nucleotide or longer) single-stranded nucleic acid (e.g., DNA or RNA) with a known sequence, the barcodes will each feature one or more handles with a sequence complementary to a region of the known target sequence. If the target strand is present in the sample solution, it will eventually connect two barcodes and form a dimer (FIG. 18A).

If the target strand is too short for stably attaching two barcode handles (e.g., shorter than 30 nucleotides), an additional step may be required. An auxiliary nucleic acid strand for every target in the conformation of a hairpin is present in solution. This auxiliary strand has a toehold which specifically recognizes the target strand and upon detection opens the hairpin. The now opened hairpin displays two previously sequestered binding domains that allow binding of two corresponding barcodes (FIG. 18B).

The auxiliary strand may be part of one of the barcode handles. Binding of the target opens the hairpin and reveals a domain that subsequently binds a dimer reporter (FIG. 18D).

### Barcode Types

Barcodes are intensity-based. Depending on the detection method, they may be either ratio-based or use absolute intensities, for example. Smaller barcodes may feature faster diffusion rates and therefore render the labeling more efficient. High barcode concentration may do the same. Barcodes should have sufficient signal strength to be detectable by the desired instrument. They should feature sufficient multiplexing capacity for the desired target pool. Furthermore, the barcodes should be specifically labeled with the target sequences.

Possible barcodes that may be used for dimerization include DNA based metafluorophores, quantum dots and fluorescent beads. Dimer reporters (see below) additionally include nanoparticles (e.g., gold, silver and diamond) and magnetic beads.

### Two Barcode Species

It may be preferred, in some embodiments, to use two distinct barcode species. One species may be used for identification of the target (“identification barcode”), the second target indicating successful dimerization (“dimer reporter”) (FIG. 18C). The identification barcode may feature two colors (e.g., red and blue), thereby allowing for combinatorial intensity barcoding. Every target may correspond to one barcode, detected by the specific barcode handle sequence. The dimer reporter may use a single color (e.g., green) not used by the identification barcode. Upon detection of all three colors (e.g., in a single spot, at a single time point), a dimer is recognized, and by analyzing the barcodimg colors, the target is identified.

### Flow Cytometry

Flow cytometry features high throughput of cells, droplets and beads. With sufficiently sized barcodes or sufficient resolution of the instrument, dimers may be visualized by front- and side-scattering, without relying on fluorescence. In such embodiments, the whole fluorescent spectrum can be used for barcodes. Reporter dimers may be non-fluorescent nanoparticles (e.g., gold particles) that scatter. In combination with a positive fluorescent signal from the identification barcode, a dimer, and thus a target, is detected.

### Fluorescence Correlation Spectroscopy (FCS)

FCS and Alternating Laser EXcitation (ALEX) allows rapid probing of a target solution with good statistics. As provided herein, monomers must be fluorescent and small for rapid diffusion. FCS/ALEX can detect single barcode duplexes based on nucleic acid nanostructures, even with only few dye molecules attached.

### Protein Detection

If the target is large enough it may serve as a dimer reporter itself.

### Beads and Barcodes

If the dimer reporter is a large microsphere or a magnetic bead, the reporters can be easily retrieved from solution after reacting. Barcodes that are not dimerized will remain in solution. After surface deposition of the beads, the attached barcodes can lie read out, wherever present, thereby target strands can be dentified.

### Quantification

Estimates of target concentrations can be made by having a known target strand with defined concentration in solution and comparing yields. Dimer/monomer ratios may also indicate concentrations.

Given an excess of barcodes, the ratios of detected targets must correspond to the target ratios in the probe solution.

### Materials and Methods

### Materials

Unmodified DNA oligonucleotides were purchased from Integrated DNA Technologies. Fluorescently modified DNA oligonucleotides were purchased from Biosynthesis. Streptavidin was purchased from Invitrogen (Catalog number: S-888). Albumin, biotin labeled bovine (BSA-biotin) was obtained from Sigma Aldrich (Catalog Number: A8549). Glass slides and coverslips were purchased from VWR. M13mp18 scaffold was obtained from New England Biolabs. ‘Freeze N Squeeze’ columns were ordered from Bio-Rad.

Two buffers were used for sample preparation and imaging:

Buffer A (10 mM Tris-HCl, 100 mM NaCl, 0.05% Tween-20, pH 8).

Buffer B (5 mM Tris-HCl, 10 mM MgCl₂, 1 mM EDTA, 0.05% Tween-20, pH 8).

### DNA Origami Self-Assembly

Self-assembly was performed in a one-pot reaction with 20 μl total volume containing 10 nM scaffold strands (M13mp18), 100 nM folding staples and 150 nM biotinylated strands, 100 nM strands with dye-handle extension and 225 nM fluorescently-labeled anti-handles in folding buffer (1× TAE Buffer with 12.5 mM MgCl₂). The solution was heated to 65° C. for 5 min and subsequently cooled to 4° C. over the course of 1 h. DNA origami were purified by agarose gel electrophoresis (1.5% agarose, 1× TAE Buffer with 12.5 mM MgCl₂) at 4.5 V/cm for 1.5 h on ice. Gel bands were cut, crushed and filled into a ‘Freeze ‘N Squeeze’ column and spun for 5 min at 1000×g at 4° C.

### Microscopy Sample Preparation

Coverslips (No. 1.5, 18×18 mm², ≈0.17 mm thick) and microscopy slides (3×1 inch², 1 mm thick) were cleaned with Isopropanol. Flow chambers were built by sandwiching two strips of double-sided sticky tape between coverslip and glass slide, resulting in a channel with ˜20 μl volume. The channel was incubated with 20 μl of 1 mg/ml BSA-Biotin solution in Buffer A (10 mM Tris-HCl, 100 mM NaCl, 0.05% Tween-20, pH 8) for 2 min. The chamber was subsequently washed with 40 μl Buffer A and then incubated with 20 μl of 0.5 mg/ml Streptavidin solution in Buffer A for 2 min. Next, a buffer exchange was performed by washing the chamber with 40 μl Buffer A and then with 40 μl Buffer B (5 mM Tris-HCl, 10 mM MgCl₂, 1 mM EDTA, 0.05% Tween-20, pH 8). Finally, 20 μl Buffer B with ˜300 pM DNA origami metafluorophores were added and incubated for 2 min and subsequently washed with 40 μl Buffer B. Finally the chamber was sealed with epoxy before imaging.

### Triggered Assembly on a Surface

Capture (CAP) and trigger (T) strands were annealed in a thermocycler directly before adding to the sample at 1 μM in 1× TAE with 12.5 mM MgCl₂ with 0.05% Tween20 (85° C. for 5 min, gradient from 85° C. to 10° C. in 15 min). Hairpin (HP) strands were annealed in a thermocycler directly before adding to the sample at 1 μM in 1× TAE with 12.5 mM MgCl₂ (85° C. for 5 min, gradient from 85° C. to 10° C. in 15 min). A flow chamber (see above) was prepared with three layers of sticky tape, resulting in ˜60 μl volume. The chamber was then incubated with 60 μl of 1 mg/ml BSA-Biotin solution in Buffer A for 2 min and then washed with 120 μl Buffer A. Next, the chamber was incubated with 60 μl of 0.5 mg/ml Streptavidin solution in Buffer A for 2 min, followed by a washing step with 120 μl Buffer A. Subsequently, a buffer-exchange was performed by adding 120 μl Buffer C (1× TAE with 12.5 mM MgCl₂ with 0.05% Tween-20). Then 60 μl Buffer C with 25 pM annealed CAP-T duplexes were added and incubated for 1 min. The chamber was washed with 120 μl Buffer C and incubated with 60 μl of 100 pM DNA origami standards for 2 min. After washing with 120 μl Buffer C, 60 μl of Buffer C with 30 nM annealed HP was added. After 20 min the chamber was washed with 120 μl Buffer C. HP incubation was repeated 3 times. Finally, the chamber was washed with 120 μl Buffer C and sealed with epoxy before imaging.

### Triggered Assembly in Solution and Gel Assay

Triggered assembly of triangles for the gel assay was performed in a one-pot reaction. Capture strands (CAP), trigger strands (T) and fluorescently labeled hairpins (HP) were added in varying stoichiometric ratios to a total volume of 40 μl. CAP strands were at a final concentration of 100 nM, T strands at 110 nM and HP strands at 550 nM (5×), 1.325 μM (12×) or 2.2 μM (20×). Strands were diluted in 1× TAE with 12.5 mM MgCl2. HP strands were annealed in a thermo cycler directly before adding to the triggered assembly reaction at 10 μM in 1× TAE with 12.5 mM MgCl₂ (85° C. for 5 min, gradient from 85° C. to 10° C. in 15 min). The control sample did not contain the T strand but HP strands at 1.325 μM (12×). Assembly was performed in low retention PCR tubes at either 30 C or at 24 C for 2 h each.

Gel electrophoresis was performed using a 2% agarose gel in 1× TAE with 12.5 mM MgCl₂, with 4.5 V/cm for 3 h on ice. Gel was scanned with a Typhoon scanner.

### Image Acquisition Parameters

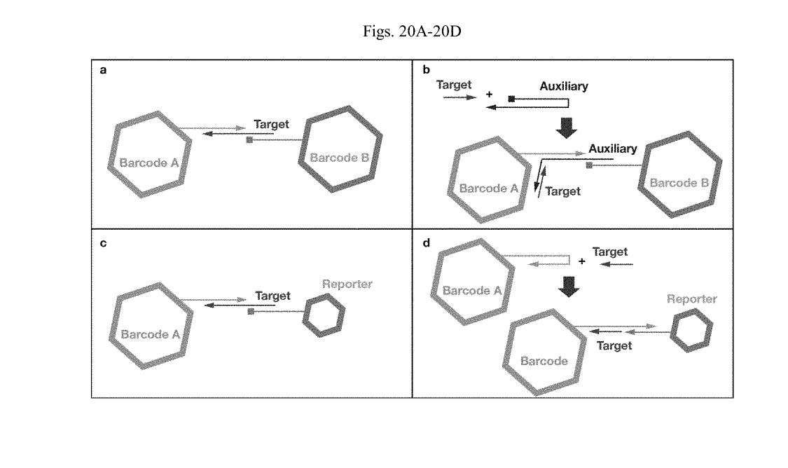

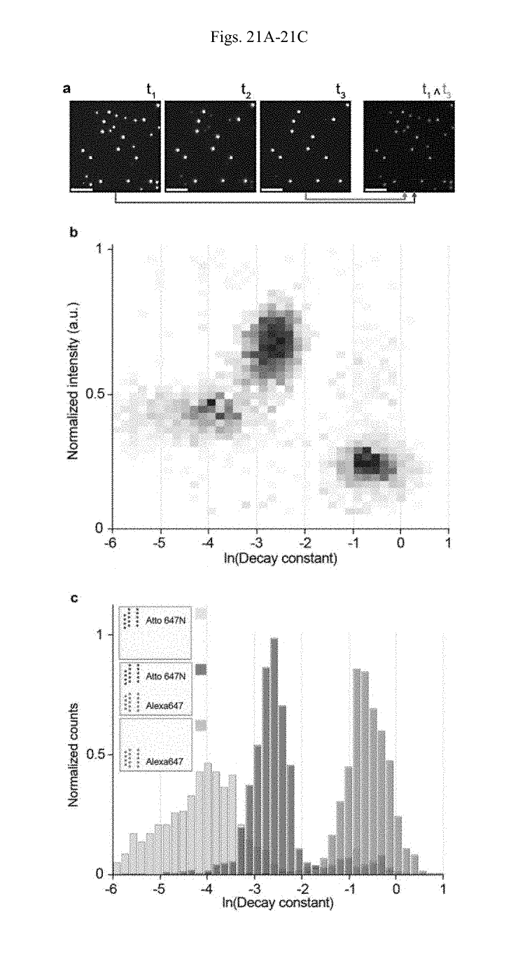

FIGS. 1, 2 3, and 20A: 10 s integration time and 60% LED Power. FIG. 20B: 5 s integration time and 60% LED Power. The decay constant was determined by acquiring a series of 10 consecutive frames and fitting the intensity vs. time trace with a single exponential decay function.

### Multiplexed Nucleic Acid Detection

Incubation was performed at room temperature in SSC-based hybridization buffer (4×SSC, 5× Denhardt's solution, 5% dextran sulfate, 0.1% Tween 20, 0.1 mg/ml salmon sperm DNA). Flow chamber volumes were designed to be ˜5 μl. Data acquisition was performed on a Zeiss LSM 780 confocal microscope.

### Optical Setup

DNA origami-based metafluorophore imaging was performed on a Zeiss Axio Observer Z1 Inverted Fluorescence Microscope with Definite Focus and a Zeiss Colibri LED illumination system (ATTO 488: 470 nm, Cy3: 555 nm, ATTO 647N: 625 nm). A Zeiss Plan-apochromat (63×/1.40 Oil) oil-immersion objective and a Hamamatsu Orca-Flash 4.0 sCMOS camera was used.
ATTO 488: Zeiss filter set 38: (BP 470/40, FT 495, BP 525/50).
Cy3: Zeiss Filter Set 43 (BP 545/25, FT 570, BP 605/70).
ATTO 647N: Zeiss filter set 50 (BP 640/30, FT 660, BP 690/50).

Triggered assembly imaging was carried out on an inverted Nikon Eclipse Ti microscope using a Nikon TIRF illuminator with an oil-immersion objective (CFI Apo TIRF 100×, numerical aperture (NA) 1.49, oil).
Lasers: 488 nm (200 mW nominal, Coherent Sapphire), 561 nm (200 mW nominal, Coherent Sapphire) and 647 nm (300 mW nominal, MBP Communications).
Camera: iXon X3 DU-897 EMCCD (Andor Technologies)
Excitation filters: (ZT488/10, ZET561/10 and ZET640/20, Chroma Technology)
Multiband beam splitter: (ZT488rdc/ZT561rdc/ZT640rdc, Chroma Technology)
Emission filters: (ET525/50m, ET600/50m and ET700/75m, Chroma Technology)

### Spot Detection, Intensity Analysis (Software)

After image acquisition, spot-detection was performed using a custom LabVIEW script [REF 2014 NatMeth]. The spot detection results in a coordinate list, which is fed into a MATLAB-based intensity analysis script. Here, 2D Gaussians are fitted within a 10×10 px² area around the center of the spots. The volume of the 2D Gaussian is proportional to the photon count and is thereby defined as intensity. Finally, one obtains a molecule-list with both, spatial coordinates and corresponding intensity values.

### Barcode Identification (Software)

All intensity values are plotted as a histogram and the local maxima (peaks) are fitted with Gaussians. Based on the intersections of these fits, the distinct intensity-level intervals can be determined.

Overlapping regions in between two peaks have to be identified and barcodes with a corresponding intensity have to be classified as unqualified. To identify the overlapping interval between two peaks, the height of the intersection (x counts) of the corresponding fits is determined. By determining the intersections of the two Gaussians with half the height of their intersection (x/2 counts), the overlapping interval is defined.

After removing the spots with unqualified intensities, the intensity values in the molecule-list are replaced with barcode-level indicators. Individual barcodes are identified by combining spots from the three molecule-lists (corresponding to the three recorded colors), which are in close proximity (i.e. <500 nm).

### Triggered Assembly (Software)

Triggered assembly evaluation was performed by determining spot coordinates and spot intensities as described above. Colocalizations of Alexa 647 and Cy3 spots were grouped as triangles (light gray) and Atto 488 and Cy3 colocalizations as DNA origami (dark gray). Plotting the two groups together results in FIG. 4C.

### Additional Sequences

M13mp18 scaffold sequence:
(SEQ ID NO: 185)
TTCCCTTCCTTTCTCGCCACGTTCGCCGGCTTTCCCCGTCAAGCTCTAAA
TCGGGGGCTCCCTTTAGGGTTCCGATTTAGTGCTTTACGGCACCTCGACC
CCAAAAAACTTGATTTGGGTGATGGTTCACGTAGTGGGCCATCGCCCTGA
TAGACGGTTTTTCGCCCTTTGACGTTGGAGTCCACGTTCTTTAATAGTGG
ACTCTTGTTCCAAACTGGAACAACACTCAACCCTATCTCGGGCTATTCTT
TTGATTTATAAGGGATTTTGCCGATTTCGGAACCACCATCAAACAGGATT
TTCGCCTGCTGGGGCAAACCAGCGTGGACCGCTTGCTGCAACTCTCTCAG
GGCCAGGCGGTGAAGGGCAATCAGCTGTTGCCCGTCTCACTGGTGAAAAG
AAAAACCACCCTGGCGCCCAATACGCAAACCGCCTCTCCCCGCGCGTTGG
CCGATTCATTAATGCAGCTGGCACGACAGGTTTCCCGACTGGAAAGCGGG
CAGTGAGCGCAACGCAATTAATGTGAGTTAGCTCACTCATTAGGCACCCC
AGGCTTTACACTTTATGCTTCCGGCTCGTATGTTGTGTGGAATTGTGAGC
GGATAACAATTTCACACAGGAAACAGCTATGACCATGATTACGAATTCGA
GCTCGGTACCCGGGGATCCTCTAGAGTCGACCTGCAGGCATGCAAGCTTG
GCACTGGCCGTCGTTTTACAACGTCGTGACTGGGAAAACCCTGGCGTTAC
CCAACTTAATCGCCTTGCAGCACATCCCCCTTTCGCCAGCTGGCGTAATA
GCGAAGAGGCCCGCACCGATCGCCCTTCCCAACAGTTGCGCAGCCTGAAT
GGCGAATGGCGCTTTGCCTGGTTTCCGGCACCAGAAGCGGTGCCGGAAAG
CTGGCTGGAGTGCGATCTTCCTGAGGCCGATACTGTCGTCGTCCCCTCAA
ACTGGCAGATGCACGGTTACGATGCGCCCATCTACACCAACGTGACCTAT
CCCATTACGGTCAATCCGCCGTTTGTTCCCACGGAGAATCCGACGGGTTG
TTACTCGCTCACATTTAATGTTGATGAAAGCTGGCTACAGGAAGGCCAGA
CGCGAATTATTTTTGATGGCGTTCCTATTGGTTAAAAAATGAGCTGATTT
AACAAAAATTTAATGCGAATTTTAACAAAATATTAACGTTTACAATTTAA
ATATTTGCTTATACAATCTTCCTGTTTTTGGGGCTTTTCTGATTATCAAC
CGGGGTACATATGATTGACATGCTAGTTTTACGATTACCGTTCATCGATT
CTCTTGTTTGCTCCAGACTCTCAGGCAATGACCTGATAGCCTTTGTAGAT
CTCTCAAAAATAGCTACCCTCTCCGGCATTAATTTATCAGCTAGAACGGT
TGAATATCATATTGATGGTGATTTGACTGTCTCCGGCCTTTCTCACCCTT
TTGAATCTTTACCTACACATTACTCAGGCATTGCATTTAAAATATATGAG
GGTTCTAAAAATTTTTATCCTTGCGTTGAAATAAAGGCTTCTCCCGCAAA
AGTATTACAGGGTCATAATGTTTTTGGTACAACCGATTTAGCTTTATGCT
CTGAGGCTTTATTGCTTAATTTTGCTAATTCTTTGCCTTGCCTGTATGAT
TTATTGGATGTTAATGCTACTACTATTAGTAGAATTGATGCCACCTTTTC
AGCTCGCGCCCCAAATGAAAATATAGCTAAACAGGTTATTGACCATTTGC
GAAATGTATCTAATGGTCAAACTAAATCTACTCGTTCGCAGAATTGGGAA
TCAACTGTTATATGGAATGAAACTTCCAGACACCGTACTTTAGTTGCATA
TTTAAAACATGTTGAGCTACAGCATTATATTCAGCAATTAAGCTCTAAGC
CATCCGCAAAAATGACCTCTTATCAAAAGGAGCAATTAAAGGTACTCTCT
AATCCTGACCTGTTGGAGTTTGCTTCCGGTCTGGTTCGCTTTGAAGCTCG
AATTAAAACGCGATATTTGAAGTCTTTCGGGCTTCCTCTTAATCTTTTTG
ATGCAATCCGCTTTGCTTCTGACTATAATAGTCAGGGTAAAGACCTGATT
TTTGATTTATGGTCATTCTCGTTTTCTGAACTGTTTAAAGCATTTGAGGG
GGATTCAATGAATATTTATGACGATTCCGCAGTATTGGACGCTATCCAGT
CTAAACATTTTACTATTACCCCCTCTGGCAAAACTTCTTTTGCAAAAGCC
TCTCGCTATTTTGGTTTTTATCGTCGTCTGGTAAACGAGGGTTATGATAG
TGTTGCTCTTACTATGCCTCGTAATTCCTTTTGGCGTTATGTATCTGCAT
TAGTTGAATGTGGTATTCCTAAATCTCAACTGATGAATCTTTCTACCTGT
AATAATGTTGTTCCGTTAGTTCGTTTTATTAACGTAGATTTTTCTTCCCA
ACGTCCTGACTGGTATAATGAGCCAGTTCTTAAAATCGCATAAGGTAATT
CACAATGATTAAAGTTGAAATTAAACCATCTCAAGCCCAATTTACTACTC
GTTCTGGTGTTTCTCGTCAGGGCAAGCCTTATTCACTGAATGAGCAGCTT
TGTTACGTTGATTTGGGTAATGAATATCCGGTTCTTGTCAAGATTACTCT
TGATGAAGGTCAGCCAGCCTATGCGCCTGGTCTGTACACCGTTCATCTGT
CCTCTTTCAAAGTTGGTCAGTTCGGTTCCCTTATGATTGACCGTCTGCGC
CTCGTTCCGGCTAAGTAACATGGAGCAGGTCGCGGATTTCGACACAATTT
ATCAGGCGATGATACAAATCTCCGTTGTACTTTGTTTCGCGCTTGGTATA
ATCGCTGGGGGTCAAAGATGAGTGTTTTAGTGTATTCTTTTGCCTCTTTC
GTTTTAGGTTGGTGCCTTCGTAGTGGCATTACGTATTTTACCCGTTTAAT
GGAAACTTCCTCATGAAAAAGTCTTTAGTCCTCAAAGCCTCTGTAGCCGT
TGCTACCCTCGTTCCGATGCTGTCTTTCGCTGCTGAGGGTGACGATCCCG
CAAAAGCGGCCTTTAACTCCCTGCAAGCCTCAGCGACCGAATATATCGGT
TATGCGTGGGCGATGGTTGTTGTCATTGTCGGCGCAACTATCGGTATCAA
GCTGTTTAAGAAATTCACCTCGAAAGCAAGCTGATAAACCGATACAATTA
AAGGCTCCTTTTGGAGCCTTTTTTTTGGAGATTTTCAACGTGAAAAAATT
ATTATTCGCAATTCCTTTAGTTGTTCCTTTCTATTCTCACTCCGCTGAAA
CTGTTGAAAGTTGTTTAGCAAAATCCCATACAGAAAATTCATTTACTAAC
GTCTGGAAAGACGACAAAACTTTAGATCGTTACGCTAACTATGAGGGCTG
TCTGTGGAATGCTACAGGCGTTGTAGTTTGTACTGGTGACGAAACTCAGT
GTTACGGTACATGGGTTCCTATTGGGCTTGCTATCCCTGAAAATGAGGGT
GGTGGCTCTGAGGGTGGCGGTTCTGAGGGTGGCGGTTCTGAGGGTGGCGG
TACTAAACCTCCTGAGTACGGTGATACACCTATTCCGGGCTATACTTATA
TCAACCCTCTCGACGGCACTTATCCGCCTGGTACTGAGCAAAACCCCGCT
AATCCTAATCCTTCTCTTGAGGAGTCTCAGCCTCTTAATACTTTCATGTT
TCAGAATAATAGGTTCCGAAATAGGCAGGGGGCATTAACTGTTTATACGG
GCACTGTTACTCAAGGCACTGACCCCGTTAAAACTTATTACCAGTACACT
CCTGTATCATCAAAAGCCATGTATGACGCTTACTGGAACGGTAAATTCAG
AGACTGCGCTTTCCATTCTGGCTTTAATGAGGATTTATTTGTTTGTGAAT
ATCAAGGCCAATCGTCTGACCTGCCTCAACCTCCTGTCAATGCTGGCGGC
GGCTCTGGTGGTGGTTCTGGTGGCGGCTCTGAGGGTGGTGGCTCTGAGGG
TGGCGGTTCTGAGGGTGGCGGCTCTGAGGGAGGCGGTTCCGGTGGTGGCT
CTGGTTCCGGTGATTTTGATTATGAAAAGATGGCAAACGCTAATAAGGGG
GCTATGACCGAAAATGCCGATGAAAACGCGCTACAGTCTGACGCTAAAGG
CAAACTTGATTCTGTCGCTACTGATTACGGTGCTGCTATCGATGGTTTCA
TTGGTGACGTTTCCGGCCTTGCTAATGGTAATGGTGCTACTGGTGATTTT
GCTGGCTCTAATTCCCAAATGGCTCAAGTCGGTGACGGTGATAATTCACC
TTTAATGAATAATTTCCGTCAATATTTACCTTCCCTCCCTCAATCGGTTG
AATGTCGCCCTTTTGTCTTTGGCGCTGGTAAACCATATGAATTTTCTATT
GATTGTGACAAAATAAACTTATTCCGTGGTGTCTTTGCGTTTCTTTTATA
TGTTGCCACCTTTATGTATGTATTTTCTACGTTTGCTAACATACTGCGTA
ATAAGGAGTCTTAATCATGCCAGTTCTTTTGGGTATTCCGTTATTATTGC
GTTTCCTCGGTTTCCTTCTGGTAACTTTGTTCGGCTATCTGCTTACTTTT
CTTAAAAAGGGCTTCGGTAAGATAGCTATTGCTATTTCATTGTTTCTTGC
TCTTATTATTGGGCTTAACTCAATTCTTGTGGGTTATCTCTCTGATATTA
GCGCTCAATTACCCTCTGACTTTGTTCAGGGTGTTCAGTTAATTCTCCCG
TCTAATGCGCTTCCCTGTTTTTATGTTATTCTCTCTGTAAAGGCTGCTAT
TTTCATTTTTGACGTTAAACAAAAAATCGTTTCTTATTTGGATTGGGATA
AATAATATGGCTGTTTATTTTGTAACTGGCAAATTAGGCTCTGGAAAGAC
GCTCGTTAGCGTTGGTAAGATTCAGGATAAAATTGTAGCTGGGTGCAAAA
TAGCAACTAATCTTGATTTAAGGCTTCAAAACCTCCCGCAAGTCGGGAGG
TTCGCTAAAACGCCTCGCGTTCTTAGAATACCGGATAAGCCTTCTATATC
TGATTTGCTTGCTATTGGGCGCGGTAATGATTCCTACGATGAAAATAAAA
ACGGCTTGCTTGTTCTCGATGAGTGCGGTACTTGGTTTAATACCCGTTCT
TGGAATGATAAGGAAAGACAGCCGATTATTGATTGGTTTCTACATGCTCG
TAAATTAGGATGGGATATTATTTTTCTTGTTCAGGACTTATCTATTGTTG
ATAAACAGGCGCGTTCTGCATTAGCTGAACATGTTGTTTATTGTCGTCGT
CTGGACAGAATTACTTTACCTTTTGTCGGTACTTTATATTCTCTTATTAC
TGGCTCGAAAATGCCTCTGCCTAAATTACATGTTGGCGTTGTTAAATATG
GCGATTCTCAATTAAGCCCTACTGTTGAGCGTTGGCTTTATACTGGTAAG
AATTTGTATAACGCATATGATACTAAACAGGCTTTTTCTAGTAATTATGA
TTCCGGTGTTTATTCTTATTTAACGCCTTATTTATCACACGGTCGGTATT
TCAAACCATTAAATTTAGGTCAGAAGATGAAATTAACTAAAATATATTTG
AAAAAGTTTTCTCGCGTTCTTTGTCTTGCGATTGGATTTGCATCAGCATT
TACATATAGTTATATAACCCAACCTAAGCCGGAGGTTAAAAAGGTAGTCT
CTCAGACCTATGATTTTGATAAATTCACTATTGACTCTTCTCAGCGTCTT
AATCTAAGCTATCGCTATGTTTTCAAGGATTCTAAGGGAAAATTAATTAA
TAGCGACGATTTACAGAAGCAAGGTTATTCACTCACATATATTGATTTAT
GTACTGTTTCCATTAAAAAAGGTAATTCAAATGAAATTGTTAAATGTAAT
TAATTTTGTTTTCTTGATGTTTGTTTCATCATCTTCTTTTGCTCAGGTAA
TTGAAATGAATAATTCGCCTCTGCGCGATTTTGTAACTTGGTATTCAAAG
CAATCAGGCGAATCCGTTATTGTTTCTCCCGATGTAAAAGGTACTGTTAC
TGTATATTCATCTGACGTTAAACCTGAAAATCTACGCAATTTCTTTATTT
CTGTTTTACGTGCAAATAATTTTGATATGGTAGGTTCTAACCCTTCCATT
ATTCAGAAGTATAATCCAAACAATCAGGATTATATTGATGAATTGCCATC
ATCTGATAATCAGGAATATGATGATAATTCCGCTCCTTCTGGTGGTTTCT
TTGTTCCGCAAAATGATAATGTTACTCAAACTTTTAAAATTAATAACGTT
CGGGCAAAGGATTTAATACGAGTTGTCGAATTGTTTGTAAAGTCTAATAC
TTCTAAATCCTCAAATGTATTATCTATTGACGGCTCTAATCTATTAGTTG
TTAGTGCTCCTAAAGATATTTTAGATAACCTTCCTCAATTCCTTTCAACT
GTTGATTTGCCAACTGACCAGATATTGATTGAGGGTTTGATATTTGAGGT
TCAGCAAGGTGATGCTTTAGATTTTTCATTTGCTGCTGGCTCTCAGCGTG
GCACTGTTGCAGGCGGTGTTAATACTGACCGCCTCACCTCTGTTTTATCT
TCTGCTGGTGGTTCGTTCGGTATTTTTAATGGCGATGTTTTAGGGCTATC
AGTTCGCGCATTAAAGACTAATAGCCATTCAAAAATATTGTCTGTGCCAC
GTATTCTTACGCTTTCAGGTCAGAAGGGTTCTATCTCTGTTGGCCAGAAT
GTCCCTTTTATTACTGGTCGTGTGACTGGTGAATCTGCCAATGTAAATAA
TCCATTTCAGACGATTGAGCGTCAAAATGTAGGTATTTCCATGAGCGTTT
TTCCTGTTGCAATGGCTGGCGGTAATATTGTTCTGGATATTACCAGCAAG
GCCGATAGTTTG.

### REFERENCES

1. Lichtman, J. W. & Conchello, J. A. Fluorescence microscopy. Nature methods 2, 910-919 (2005).
2. Weiss, S. Fluorescence spectroscopy of single biomolecules. Science 283, 1676-1683 (1999).
3. Joo, C., Balci, H., Ishitsuka, Y., Buranachai, C. & Ha, T. Advances in single-molecule fluorescence methods for molecular biology. Annual review of biochemistry 77, 51-76 (2008).
4. Tsien, R. Y. The green fluorescent protein. Annual review of biochemistry 67, 509-544 (1998).
5. Giepmans, B. N., Adams, S. R., Ellisman, M. H. & Tsien, R. Y. The fluorescent toolbox for assessing protein location and function. Science 312, 217-224 (2006).
6. Resch-Genger, U., Grabolle, M., Cavaliere-Jaricot, S., Nitschke, R. & Nann, T. Quantum dots versus organic dyes as fluorescent labels. Nature methods 5, 763-775 (2008).
7. Goncalves, M. S. Fluorescent labeling of biomolecules with organic probes. Chemical reviews 109, 190-212 (2009).
8. Han, M., Gao, X., Su, J. Z. & Nie, S. Quantum-dot-tagged microbeads for multiplexed optical coding of biomolecules. Nat Biotechnol 19, 631-635 (2001).
9. Levsky, J. M., Shenoy, S. M., Pezo, R. C. & Singer, R. H. Single-cell gene expression profiling. Science 297, 836-840 (2002).
10. Li, Y., Cu, Y. T. H. & Luo, D. Multiplexed detection of pathogen DNA with DNA-based fluorescence nanobarcodes. Nat Biotechnol 23, 885-889 (2005).
11. Seeman, N. C. Nucleic-Acid Junctions and Lattices. J Theor Biol 99, 237-247 (1982).
12. Rothemund, P. W. Folding DNA to create nanoscale shapes and patterns. Nature 440, 297-302 (2006).
13. Douglas, S. M. et al. Self-assembly of DNA into nanoscale three-dimensional shapes. Nature 459, 414-418 (2009).
14. Tørring, T., Voigt, N. V., Nangreave, J., Yan, H. & Gothelf, K. V. DNA origami: a quantum leap for self-assembly of complex structures. Chemical Society Reviews (2011).
15. Wei, B., Dai, M. & Yin, P. Complex shapes self-assembled from single-stranded DNA tiles. Nature 485, 623-626 (2012).
16. Ke, Y., Ong, L. L., Shih, W. M. & Yin, P. Three-dimensional structures self-assembled from DNA bricks. Science 338, 1177-1183 (2012).
17. Nicewarner-Pena, S. R. Submicrometer metallic barcodes. Science 294, 137-141 (2001).
18. Gudiksen, M. S., Lauhon, L. J., Wang, J., Smith, D. C. & Lieber, C. M. Growth of nanowire superlattice structures for nanoscale photonics and electronics. Nature 415, 617-620 (2002).
19. Braeckmans, K. et al. Encoding microcarriers by spatial selective photobleaching. Nature materials 2, 169-173 (2003).
20. Dejneka, M. J. et al. Rare earth-doped glass microbarcodes. Proceedings of the National Academy of Sciences of the United States of America 100, 389-393 (2003).
21. Geiss, G. K. et al. Direct multiplexed measurement of gene expression with color-coded probe pairs. Nat Biotechnol 26, 317-325 (2008).
22. Xiao, M. et al. Direct determination of haplotypes from single DNA molecules. Nature methods 6, 199-201 (2009).
23. Li, X. et al. Controlled fabrication of fluorescent barcode nanorods. ACS nano 4, 4350-4360 (2010).
24. Lubeck, E. & Cai, L. Single-cell systems biology by super-resolution imaging and combinatorial labeling. Nature methods 9, 743-748 (2012).
25. Lin, C. et al. Submicrometre geometrically encoded fluorescent barcodes self-assembled from DNA. Nature chemistry 4, 832-839 (2012).
26. Lubeck, E., Coskun, A. F., Zhiyentayev, T., Ahmad, M. & Cai, L. Single-cell in situ RNA profiling by sequential hybridization. Nature methods 11, 360-361 (2014).
27. Xu, H. et al. Multiplexed SNP genotyping using the Qbead system: a quantum dot-encoded microsphere-based assay. Nucleic Acids Research 31, e43 (2003).
28. Lin, C., Liu, Y. & Yan, H. Self-assembled combinatorial encoding nanoarrays for multiplexed biosensing. Nano letters 7, 507-512 (2007).
29. Livet, J. et al. Transgenic strategies for combinatorial expression of fluorescent proteins in the nervous system. Nature 450, 56-62 (2007).
30. Fournier Bidoz, S. et al. Facile and rapid one-step mass preparation of quantum-dot barcodes. Angewandte Chemie International Edition 47, 5577-5581 (2008).
31. Marcon, L. et al. ‘On-the-fly’ optical encoding of combinatorial peptide libraries for profiling of protease specificity. Molecular bioSystems 6, 225-233 (2010).
32. Elshal, M. F. & McCoy, J. P. Multiplex bead array assays: performance evaluation and comparison of sensitivity to ELISA. Methods 38, 317-323 (2006).
33. Forster, T. Zwischenmolekulare Energiewanderung and Fluoreszenz. Annalen der Physik 437, 55-75 (1948).
34. Dirks, R. M. & Pierce, N. A. Triggered amplification by hybridization chain reaction. Proceedings of the National Academy of Sciences of the United States of America 101, 15275-15278 (2004).
35. Sadowski, J. P., Calvert, C. R., Zhang, D. Y., Pierce, N. A. & Yin, P. Developmental self-assembly of a DNA tetrahedron. ACS Nano 8, 3251-3259 (2014).
36. Yin, P., Choi, H. M., Calvert, C. R. & Pierce, N. A. Programming biomolecular self-assembly pathways. Nature 451, 318-322 (2008).
37. Yurke, B., Turberfield, A. J., Mills, A. P., Jr., Simmel, F. C. & Neumann, J. L. A DNA-fuelled molecular machine made of DNA. Nature 406, 605-608 (2000).
38. Zhang, D. Y. & Seelig, G. Dynamic DNA nanotechnology using strand-displacement reactions. Nature chemistry 3, 103-113 (2011).
39. Liu, Y., Lin, C., Li, H. & Yan, H. Aptamer-directed self-assembly of protein arrays on a DNA nanostructure. Angew Chem Int Ed Engl 44, 4333-4338 (2005).
40. Rinker, S., Ke, Y., Liu, Y., Chhabra, R. & Yan, H. Self-assembled DNA nanostructures for distance-dependent multivalent ligand-protein binding. Nat Nanotechnol 3, 418-422 (2008).
41. Jungmann, R., Scheible, M. & Simmel, F. C. Nanoscale imaging in DNA nanotechnology. Wiley interdisciplinary reviews. Nanomedicine and nanobiotechnology 4, 66-81 (2012).
42. Gietl, A., Holzmeister, P., Grohmann, D. & Tinnefeld, P. DNA origami as biocompatible surface to match single-molecule and ensemble experiments. Nucleic Acids Res 40, e110 (2012).
43. Steinhauer, C., Jungmann, R., Sobey, T. L., Simmel, F. C. & Tinnefeld, P. DNA origami as a nanoscopic ruler for super-resolution microscopy. Angewandte Chemie (International ed in English) 48, 8870-8873 (2009).
44. Jungmann, R. et al. Single-Molecule Kinetics and Super-Resolution Microscopy by Fluorescence Imaging of Transient Binding on DNA Origami. Nano letters 10, 4756-4761 (2010).
45. Johnson-Buck, A. et al. Super-resolution fingerprinting detects chemical reactions and idiosyncrasies of single DNA pegboards. Nano letters 13, 728-733 (2013).
46. Iinuma, R. et al. Polyhedra self-assembled from DNA tripods and characterized with 3D DNA-PAINT. Science 344, 65-69 (2014).
47. Jungmann, R. et al. Multiplexed 3D cellular super-resolution imaging with DNA-PAINT and Exchange-PAINT. Nature methods 11, 313-318 (2014).
48. Schmied, J. J. et al. Fluorescence and super-resolution standards based on DNA origami. Nature methods 9, 1133-1134 (2012).
49. Schmied, J. J. et al. DNA origami-based standards for quantitative fluorescence microscopy. Nature protocols 9, 1367-1391 (2014).
50. Ha, T. & Tinnefeld, P. Photophysics of fluorescent probes for single-molecule biophysics and super-resolution imaging. Annual review of physical chemistry 63, 595-617 (2012).
51. Anderson, G. P. & Nerurkar, N. L. Improved fluoroimmunoassays using the dye Alexa Fluor 647 with the RAPTOR, a fiber optic biosensor. Journal of immunological methods 271, 17-24 (2002).
52. Levsky, J. M. & Singer, R. H. Fluorescence in situ hybridization: past, present and future. J Cell Sci 116, 2833-2838 (2003).
53. Raj, A., van den Bogaard, P., Rifkin, S. A., van Oudenaarden, A. & Tyagi, S. Imaging individual mRNA molecules using multiple singly labeled probes. Nature Chemical Biology 5, 877-879 (2008).
54. Choi, H. M., Beck, V. A. & Pierce, N. A. Next-generation in situ hybridization chain reaction: higher gain, lower cost, greater durability. ACS Nano 8, 4284-4294 (2014).
55. Myhrvold, C., Dai, M., Silver, P. A. & Yin, P. Isothermal self-assembly of complex DNA structures under diverse and biocompatible conditions. Nano letters 13, 4242-4248 (2013).
56. Hell, S. W. Microscopy and its focal switch. Nature methods 6, 24-32 (2009).
57. Gustafsson, M. G. et al. Three-dimensional resolution doubling in wide-field fluorescence microscopy by structured illumination. Biophysical journal 94, 4957-4970 (2008).

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

It should also be understood that, unless clearly indicated to the contrary, in any methods claimed herein that include more than one step or act, the order of the steps or acts of the method is not necessarily limited to the order in which the steps or acts of the method are recited.

All references (e.g., published journal articles, books, etc.), patents and patent applications disclosed herein are incorporated by reference with respect to the subject matter for which each is cited, which, in some cases, may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system comprising:
a nucleic acid capture strand linked to a first dye molecule;
a nucleic acid trigger strand longer than the capture strand and comprising (a) a capture domain that is complementary to the capture strand and (b) at least two concatenated domains, each of which comprises two subdomains; and
a partially double-stranded nucleic acid comprising
a single-stranded toehold domain having a nucleotide sequence complementary to one of the subdomains of the two subdomains of the concatenated domains,
a double-stranded region linked to a second dye molecule and having a nucleotide sequence complementary to the other of the two subdomains of the concatenated domains, and
a single-stranded hairpin loop having a nucleotide sequence that is complementary to the single-stranded toehold domain.

2. The system of claim 1, wherein the nucleic acid capture strand has a length of 10-100 nucleotides.

3. The system of claim 1, wherein the dye molecule is a fluorescent dye molecule.

4. The system of claim 1, wherein the nucleic acid trigger strand has a length of 100-5000 nucleotides.

5. The system of claim 4, wherein the nucleic acid trigger strand has a length of 100-1000 nucleotides.

6. The system of claim 1, wherein the capture domain has a length of 10-100 nucleotides.

7. The system of claim 1, wherein a concatenated domain of a nucleic acid trigger strand has a length of 15-100 nucleotides.

8. The system of claim 1, wherein at least one of the two subdomains of a concatenated domain has a length of 5-50 nucleotides.

9. The system of claim 1, wherein one of the two subdomains of a concatenated domain is longer than the other of the two subdomains.

10. The system of claim 1, wherein the partially double-stranded nucleic acid has a length of 20-500 nucleotides.

11. The system of claim 1, wherein the single-stranded toehold domain has a length of 5-50 nucleotides.

12. The system of claim 1, wherein the double-stranded region has a length of 10-100 nucleotides.

13. The system of claim 1, wherein the single-stranded hairpin loop has a length of 5-50 nucleotides.

14. The system of claim 1, wherein the nucleic acid capture strand is attached to a substrate.

15. The system of claim 1 further comprising at least two partially double-stranded nucleic acids.

16. The system of claim 15 further comprising at least ten partially double-stranded nucleic acids.

17. The system of claim 1, wherein at least one partially double-stranded nucleic acid is bound to the trigger nucleic acid.

18. The system of claim 16, wherein at least ten partially double-stranded nucleic acids are assembled on a single-stranded trigger nucleic acid bound to a single-stranded capture strand, thereby forming a nucleic acid nanostructure comprising at least 10 dye molecules.
